SEQUENCE LISTING (34 sequences)

SEQ ID NO: 1 - protein, 7 aa, Rattus norvegicus
FTFDNAW

SEQ ID NO: 2 - protein, 10 aa, Rattus norvegicus
IKAKSNNYAT

SEQ ID NO: 3 - protein, 6 aa, Rattus norvegicus
TRLYYD

SEQ ID NO: 4 - protein, 115 aa, Rattus norvegicus
EVQLVETGGNLVQPGKSLKLTCATSGFTFDNAWMHWVRQSPEKQLEWVAQIKAKSNNYATYYAESVKGRFTISRDDSKSSVYLQMNRLKEEDTAIYYCTRLYYDWGQGVMVTVSS

SEQ ID NO: 5 - protein, 6 aa, Rattus norvegicus
QDIGNY

SEQ ID NO: 6 - protein, 3 aa, Rattus norvegicus
RAT

SEQ ID NO: 7 - protein, 8 aa, Rattus norvegicus
LQHKQYPL

SEQ ID NO: 8 - protein, 108 aa, Rattus norvegicus
DIQMTQSPSSISVSLGDRFTFTCRASQDIGNYLSWFQQKPEKSPKLMIYRATNLEDGVPSRFSGSRSGSDYSLTINSLESEDTGIYFCLQHKQYPLTFGSGTKLEIKR

SEQ ID NO: 9 - protein, 7 aa, Rattus norvegicus
FTFSNYD

SEQ ID NO: 10 - protein, 10 aa, Rattus norvegicus
ISHSGDSTYF

SEQ ID NO: 11 - protein, 6 aa, Rattus norvegicus
HGVYTN

SEQ ID NO: 12 - protein, 122 aa, Rattus norvegicus
EVQLQESGGGLIQPGRSLKLSCAASGFTFSNYDMAWVRQAPTKGLEWVASISHSGDSTYFRASVKGRFTVSRDNAKSSLYLQMDSLRSEDTATYYCARHGVYTNYGIWFAYWGQGTLVTVSS

SEQ ID NO: 13 - protein, 6 aa, Rattus norvegicus
QDIGNY

SEQ ID NO: 14 - protein, 3 aa, Rattus norvegicus
RAT

SEQ ID NO: 15 - protein, 8 aa, Rattus norvegicus
LQHKQYPL

SEQ ID NO: 16 - protein, 108 aa, Rattus norvegicus; identical to SEQ ID NO: 8

SEQ ID NO: 17 - protein, 7 aa, Rattus norvegicus
FTFSSAW

SEQ ID NO: 18 - protein, 10 aa, Rattus norvegicus
IKGKSNNYAT

SEQ ID NO: 19 - protein, 8 aa, Rattus norvegicus
TWFGPMDA

SEQ ID NO: 20 - protein, 117 aa, Rattus norvegicus
EVQLQESGGRLVQPGKSLKLTCAASGFTFSSAWIHWVRQSPEKQLEWVAQIKGKSNNYATYYAESVKGRFTISRDDSKSSVYLQMNSLKEEDTAIYYCTWFGPMDAWGQGASVTVSS

SEQ ID NO: 21 - protein, 11 aa, Rattus norvegicus
KSLLHNNGKTF

SEQ ID NO: 22 - protein, 3 aa, Rattus norvegicus
WMS

SEQ ID NO: 23 - protein, 8 aa, Rattus norvegicus
QQFLEYPL

SEQ ID NO: 24 - protein, 113 aa, Rattus norvegicus
DIVMTQGALPNPVPSGESASITCQSSKSLLHNNGKTFLNWYLQRPGQSPQLLIYWMSTRASGVSDRFSGSGSGADFTLKISSVEAEDVGVYYCQQFLEYPLTFGSGTKLEIKR

SEQ ID NO: 25 - protein, 318 aa, Homo sapiens
MQRANHSTVTQFILVGFSVFPHLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLAFCGHKEIHHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 26 - RNA, 19 nt, Artificial
  siRNA
ggagacaccuugaugggca

SEQ ID NO: 27 - RNA, 19 nt, Artificial
  siRNA
aguaaacucuacccagaaa

SEQ ID NO: 28 - RNA, 19 nt, Artificial
  siRNA
gcagagagccaaucacucc

SEQ ID NO: 29 - RNA, 19 nt, Artificial
  siRNA
ggucgugcacuauggcuuu

SEQ ID NO: 30 - DNA, 21 bp, Artificial
  shRNA
gttcctgctgatgtacctgtt

SEQ ID NO: 31 - DNA, 21 bp, Artificial
  shRNA
tgcgctacaacgtgctcatga

SEQ ID NO: 32 - DNA, 21 bp, Artificial
  shRNA
tggctttgcctccgtcattta

SEQ ID NO: 33 - DNA, 21 bp, Artificial
  shRNA
tctgctgaaggtcggaacaag

SEQ ID NO: 34 - DNA, 21 bp, Artificial
  shRNA
acacaaggagatccaccattt

(19) United States
(12) Patent Application Publication
(10) Pub. No.: US 2019/0263906 A1
(43) Pub. Date: Aug. 29, 2019

(54) IMMUNE MODULATORS FOR REDUCING IMMUNE-RESISTANCE IN MELANOMA AND OTHER PROLIFERATIVE DISEASES

(71) Applicant: Deutsches Krebsforschungszentrum Stiftung des öffentlichen Rechts, Heidelberg (DE)

(72) Inventors: Philipp Beckhove, Regensburg (DE); Michael Boutros, Heidelberg (DE); Marco Breinig, Schriesheim (DE); Nisit Khandelwal, Martinsried (DE); Tillmann Michels, Regensburg (DE); Antonio Sorrentino, Heidelberg (DE); Valentina Volpin, Regensburg (DE)

(21) Appl. No.: 16/347,397

(22) PCT Filed: Nov. 10, 2017
(86) PCT No.: PCT/EP2017/078856

§ 371 Date: May 3, 2019

(30) Foreign Application Priority Data
Nov. 10, 2016 (EP) EP16198189.9
Nov. 10, 2016 (EP) PCT/EP2016/077288

(51) Int. Cl. C07K 16/28; C07K 14/705; A61K 31/7088; C12N 15/85; C12N 15/113; G01N 33/574; A61P 35/00; A61K 39/00

(52) U.S. Cl. CPC C07K 16/286; C07K 14/70571; A61K 31/7088; C12N 15/85; C12N 15/1138; G01N 33/57484; A61K 2039/505; A61K 39/001102; G01N 2333/726; G01N 2500/10; A61K 2039/5158; A61K 2039/585; A61P 35/00

(57) ABSTRACT

The present invention pertains to novel modulators of resistance against T-cell mediated cytotoxic immune responses. The invention provides antagonists of immune escape mechanisms and therefore offers a novel approach for treating, or aiding a treatment, of various proliferative diseases such as cancerous diseases, in particular melanoma, pancreatic cancer and colorectal cancer. The invention specifically discloses the receptor Olfactory Receptor, Family 10, Subfamily H, Member 1 (OR10H1)as a checkpoint molecule in tumor resistance against cytotoxic T-cells. Provided is the inhibition of OR10H1 expression and/or function as a strategy for enhancing tumor susceptibility to a patients T-cell mediated immune response. Provided are antigen binding constructs for the detection of the OR10H1 protein, as well as inhibitory compounds, such as siRNA/shRNA molecules targeting OR10H1 and anti-OR10H1 antibodies, forimpairing the immune escape mediated by OR10H1. The invention furthermore provides screening methods for the identification of novel cancer therapeutics based on the modulation OR10H1 expression/function, diagnostic methods for the detection of immune resistance of a tumor to cytotoxic T-cell responses, as well as pharmaceutical compositions and diagnostic kits for performing these methods.

The present invention pertains to novel modulators of resistance against T-cell mediated cytotoxic immune responses. The invention provides antagonists of immune escape mechanisms and therefore offers a novel approach for treating, or aiding a treatment, of various proliferative diseases such as cancerous diseases, in particular melanoma, pancreatic cancer and colorectal cancer. The invention specifically discloses the receptor Olfactory Receptor, Family 10, Subfamily H, Member 1 (OR10H1) as a checkpoint molecule in tumor resistance against cytotoxic T-cells. Provided is the inhibition of OR10H1 expression and/or function as a strategy for enhancing tumor susceptibility to a patients T-cell mediated immune response. Provided are antigen binding constructs for the detection of the OR10H1 protein, as well as inhibitory compounds, such as siRNA/shRNA molecules targeting OR10H1 and anti-OR10H1 antibodies, for impairing the immune escape mediated by OR10H1. The invention furthermore provides screening methods for the identification of novel cancer therapeutics based on the modulation OR10H1 expression/function, diagnostic methods for the detection of immune resistance of a tumor to cytotoxic T-cell responses, as well as pharmaceutical compositions and diagnostic kits for performing these methods.

Peripheral immune tolerance is important to prevent autoimmune disorders. However, tumor cells use immune checkpoints to prevent immune recognition (Zitvogel et al, Nat Rev Immunol. 2006; 6:715-727; Rabinovich et al, Annu Rev Immunol. 2007; 25:267-296). Blocking antibodies against surface-expressed immune-regulatory proteins, such as CTLA4 and PD-L1 (Chambers et al, Annu Rev Immunol. 2001; 19:565-594; Blank et al, Cancer Res. 2004; 64:1140-1145), boost anti-tumor immunity and are successfully applied in clinical trials (van Elsas et al, J Exp Med. 1999; 190:355-366; Weber, Oncologist. 2007; 12:864-872; Brahmer et al, New Engl J Med. 2012; 366:2455-2465; Topalian et al, New Engl J Med. 2012; 366:2443-2454). Still, treatment unresponsiveness is frequent among patients (Topalian et al, New Engl J Med. 2012; 366:2443-2454), indicating that other immune-checkpoint pathways may be active. Therefore, successful cancer immunotherapy requires a systematic delineation of the entire immune-regulatory circuit—the ‘immune modulatome’—expressed on tumors (Woo et al, Cancer Res. 2012; 72:917-927; Berrien-Elliott et al, Cancer Res. 2013; 73 :605-616).

A comprehensive detection of immune-checkpoint molecules has been technically challenging due to the lack of robust high-throughput assays that enable a qualitative and quantitative analysis of heterologous interactions between tumor cells and T cells. Screening strategies before have relied on interferon-gamma (IFN-γ) release as an indicator of anti-tumor NK cell activity (Hill & Martins, Methods. 2006; 38:312-316; Bellucci et al, J Clin Investig. 2012; 122:2369-2383). However, IFN-γ secretion alone by immune cells does not always correlate with cellular cytotoxicity (Bachmann et al, Eur J Immunol. 1999; 29:291-299; Slifka et al, Nature. 1999; 401:76-79).

Olfactory receptors (ORs) are members of the seven transmembrane G protein-coupled Receptor (GPCR) class A (Rhodopsin-like). They sense the chemical environment and can be distinguished by the chemo stimuli to which they respond. Olfactory receptors signal mainly via a unique G protein-coupled adenylyl cyclase cascade. Subsequently, cAMP is the key messenger of olfactory G protein signaling. Olfactory signaling leads to the specific cAMP production by adenylyl cyclase type III. AC3 in turn is activated by the olfactory-restricted G protein alpha subunit GαOlf. It was shown that olfactory receptors could couple in vitro to Gαs and Gα15 G proteins, which might alter the specificity of the receptor. Furthermore, olfactory receptors can signal via other mechanisms. Olfactory receptor activation leads to production of cGMP, opens cyclic nucleotide-gated channels (CNC) by cAMP and cGMP, stimulates the production of Inositol-1,4,5-trisphosphate (IP3) and increases influx of calcium.

Olfactory receptors are a highly divergent group of receptors ranging in a length of 300-350 amino acids, depending on the length of the N- and C-terminal stretches. They are coded by single coding-exon genes, but exons in the 5′ untranslated region may undergo alternate splicing. Due to a high number of degenerated pseudogenes only about 390 human functional OR genes are known (compared to 855 OR genes in total). Human ORs are organized in 18 families (sequence similarity >40%) and around 300 subfamilies (similarity >60%). As mentioned before ORs contain seven hydrophobic membrane-spanning domains and belong to the GPCR class A. Several characteristic conserved amino acids motifs distinguish ORs from other GPCRs (e.g. extracellular NXS/T consensus for N-linked glycosylation). Interestingly, published data suggest that the transmembrane domains (e.g. amino acids in TM3, TM5 and TM6) are essential for the specificity of odorant binding pockets.

There is a need in the art for novel approaches to circumvent tumor immune escape mechanisms and to render tumors more susceptible for the immune system. The present invention seeks to provide novel therapeutic compounds, as well as therapeutic and diagnostic approaches and methods involving such compounds, that are able to modulate a host's immune response, in particular to strengthen a cytotoxic T cell response against tumor cells. Furthermore, the invention seeks to provide novel strategies to diagnose tumor resistance to immune response and screening approaches for the identification of compounds that are useful in cancer treatment.

The above problem is solved in a first aspect by a compound for use in the treatment of a disease of a patient, wherein the compound is a modulator of the expression, function and/or stability of Olfactory Receptor, Family 10, Subfamily H, Member 1 (OR10H1), or of a variant of OR10H1. The treatment of the present invention is preferably a method for treating a disease in a subject, comprising a step of administering to the subject a therapeutically effective amount of a modulator of the expression, function and/or stability of OR10H1, or of a variant of OR10H1.

The term “olfactory receptor family 10” pertains to a family of olfactory receptor proteins containing 29 subfamilies, wherein the subfamily H consists of five functional OR genes (no pseudogenes). Olfactory receptor family 10 subfamily H member 1 (OR10H1) is also known as AC004510, OR19-27, HSOR19.4.4, ORL733, ORL525 (HUGO Gene Nomenclature Committee symbol: HGNC:8172). The OR10H1 gene (chromosome 19: 15,807,003-15,808,126; GRCh38:CM000681.2) is 1124 base pairs long and codes a protein of 318 amino acids (SEQ ID NO: 25, see below), UniProtKB identifier Q9Y4A (Sequence version 1 of 1 Nov. 1999; Entry version 126 of 5Oct. 2016).

The terms “OR10H1-protein” or “protein of OR10H1” as used in context of the herein disclosed invention shall pertain to a protein (such as a full-length protein, fusion protein or partial protein) comprising a sequence as shown in SEQ ID NO: 25. The terms shall also refer to a protein comprising the amino acid sequence according to SEQ ID NO: 25 with any protein modifications. Such protein modifications preferably do not alter the amino acid sequence of the polypeptide chain, but constitute a functional group, which is conjugated to the basic amino acid polymer chain. Protein modifications in context of the invention may be selected from a conjugation of additional amino acid sequences to the OR10H1 amino acid chain, such as ubiquitination, sumolation, neddylation, or similar small protein conjugates. Other protein modifications include, but are not limited to, glycosylation, methylation, lipid-conjugation, or other natural or artificial post-translational modifications known to the skilled person. The terms “protein of a variant of OR10H1” and the like, shall have the corresponding meaning with respect to a variant of OR10H1.

The terms “OR10H1-mRNA” or “mRNA of OR10H1” as used in context of the herein disclosed invention shall pertain to a messenger ribonucleic acid (such as a full-length mRNA, fusion mRNA or partial mRNA, and/or splice-variants thereof) comprising a region encoding for an amino acid sequence as shown in SEQ ID NO: 25. The terms shall also refer to an mRNA comprising a region encoding for the amino acid sequence according to SEQ ID NO: 25 with any codon or nucleotide modifications. Such modifications preferably would not alter the amino acid sequence of the encoded polypeptide chain. The terms “mRNA of a variant of OR10H1” and the like, shall have the corresponding meaning with respect to a variant of OR10H1.

A variant of OR10H1 is, in some embodiments, a protein comprising an amino acid sequence having at least 60%, 70%, 80%, 90%, preferably at least 80% such as at least 90% sequence identity to SEQ ID NO: 25, and most preferably at least 95% (such as at least 98%) sequence identity to SEQ ID NO: 25 (the human OR10H1 amino acid sequence). In one preferred embodiment of the invention, the variant of OR10H1 comprises an amino acid sequence with at least 80% sequence identity to the amino acid sequence shown in SEQ ID NO: 25.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of OR10H1 to another protein/gene, the percentage identity can be determined by the Blast searches supported at the Human Olfactory Data Explorer (eg, https://genome.weizmann.acil/cgi-bin/horde/blastHorde.p1); in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

A variant of OR10H1 is, in certain embodiments, a functional variant of OR10H1 protein. In other embodiments of the invention, the variant of OR10H1 is selected from the group consisting of an ortholog or paralog of OR10H1, and a functional fragment of an OR10H1 protein.

The term “ortholog” refers to homologs in different species that evolved from a common ancestral gene by speciation. Typically, orthologs retain the same, essentially the same or similar function despite differences in their primary structure (mutations). The term “paralog” refers to homologs in the same species that evolved by genetic duplication of a common ancestral gene. In many cases, paralogs exhibit related but not always similar function. The term “splice variant” refers to a related protein expressed from the same genomic locus as a parent protein, but having a different amino acid sequence based on a different exon composition due to differential splicing of the transcribed RNA.

In context of the present invention the term “subject” or “patient” preferably refers to a mammal, such as a mouse, rat, guinea pig, rabbit, cat, dog, monkey, or preferably a human, for example a human patient. The subject of the invention may be at danger of suffering from a proliferative disease such as a cancer or a tumor disease, or suffer from a cancer or tumor disease, preferably, wherein the tumor disease is a tumor having a resistance or increased resistance to the host's (the patient's) immune response. A preferred immune response in context of the invention is a cell-mediated immune response such as a cytotoxic T-cell response. A more detailed description of medical indications relevant in context of the invention is provided herein elsewhere.

The term “modulator” in context of the present invention shall include inhibitors/antagonists. A preferred embodiment of the invention pertains to inhibitors/antagonists as modulators of the expression, function and/or stability of Olfactory Receptor, Family 10, Subfamily H, Member 1 (OR10H1), or of a variant of OR10H1.

A “modulator of expression, function and/or stability of OR10H1, or of a variant of OR10H1”, or grammatically similar expressions, in context of the invention may be any compound that affects, for example when an inhibitor/antagonist impairs or interferes with, the expression, function and/or stability of OR10H1, or of a variant of OR10H1, in particular the expression, function and/or stability of protein of OR10H1 or the variant, and/or the expression, function and/or stability of mRNA of OR10H1 or the variant.

Preferred modulators are, in context of the invention, inhibitors or antagonists that inhibit (eg, impair or interfere with) OR10H1 or its variant's expression, function and/or stability, in particularly specifically and/or selectively, in cells showing a pathological form of cell proliferation, growth or survival. A preferred example is a cell that has become a tumor cell, or originates from a tumor cell.

In one embodiment, the modulator of expression, function and/or stability of OR10H1, or of a variant of OR10H1, may modulate OR10H1, or the variant of OR10H1, via a direct interaction (such as non-covalent and covalent binding) between the modulator and the OR10H1 protein, or a protein of a OR10H1 variant, their RNA transcripts or coding genomic loci. In other embodiments, the invention also includes modulators of OR10H1 expression, function and/or stability that interact with one or more other components of the OR10H1-mediated immune modulatory mechanism and signalling pathway as disclosed herein and/or with one or more other genes that control the expression, function and/or stability of protein or mRNA of OR10H1, or of the variant of OR10H1. For example, a modulator of the invention may inhibit the expression, function and/or stability of OR10H1, or of a variant of OR10H1, binding directly to a protein of OR10H10 or the variant, and so for example inhibit the function of OR10H1 or the variant (such as a modulator that is an inhibitory antibody against protein of OR10H1 or of the variant), or may bind directly to mRNA of OR10H10 or the variant, and so for example inhibit the expression of OR10H1 or the variant (such as a modulator that is an anti-sense nucleotide molecule against mRNA of OR10H1 or of the variant). Alternatively, the modulator of the invention may inhibit the expression, function and/or stability of another gene that itself modulates the expression, function and/or stability of OR10H1, or of a variant of OR10H1; for example, a modulator that is an anti-sense nucleotide molecule against mRNA of an transcription factor for or repressor protein of OR10H1 or the variant. Mechanisms by which such modulation may be brought about, and/or the effects of such modulation, can include one or more of those as described elsewhere herein.

Particularly preferred modulators (in particular inhibitors/antagonists) of expression, function and/or stability of OR10H1, or of a variant of OR10H1, are in certain embodiments the following specific molecules and/or molecular classes.

The modulator of expression, function and/or stability of OR10H1, or of a variant of OR10H1 of the invention is in some embodiments selected from a compound which is polypeptide, peptide, glycoprotein, a peptidomimetic, an antigen binding construct (for example, an antibody, antibody-like molecule or other antigen binding derivative, or an antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA. The basic rules for the design of CRISPR/Cas9 mediated gene editing approaches are known to the skilled artisan and for example reviewed in Wiles MV et al (Mamm Genome. 2015 October; 26(9-10):501-10) or in Savić N and Schwank G (Transl Res. 2016 February; 168:15-21).

In one preferred embodiment the modulator of expression, function and/or stability of OR10H1, or of a variant of OR10H1, is an antigen binding construct such as described in detail herein below, more preferably such an antigen binding construct that binds to, such as specifically binds to, protein of OR10H1, or a variant of OR10H1.

In another preferred embodiment the modulator of expression, function and/or stability of OR10H1, or of a variant of OR10H1, is an anti-sense nucleotide molecule such as described in detail herein below, more preferably one that binds to, such as specifically binds to, a nucleic acid that encodes or regulates the expression of OR10H1, or of a variant of OR10H1, or alternatively more preferably one that binds to, such as specifically bind to, a nucleic acid that encodes or (regulates the expression of a gene that controls the expression, function and/or stability of) OR10H1, or of a variant of OR10H1.

As used herein, the terms “inhibitor of OR10H1 expression” and the like (including similarly, “antagonist of OR10H1 expression” and the like) shall relate to any of the herein disclosed modulators (for example, the antigen binding constructs or anti-sense molecules described herein), which has an antagonistic activity toward the expression of an OR10H1 protein, such that it impairs, suppresses, reduces and/or lowers the expression of an OR10H1 protein such as may be determined by measuring an amount (or change in an amount) of OR10H1 protein or OR10H1 mRNA. The term “expression” means in this context the cellular process of transcribing a gene into an mRNA and the following translation of the mRNA into a protein. “Gene expression” therefore may refer only to the generation of mRNA, irrespectively from the fate of the so produced mRNA, or alternatively/additionally to the translation of the expressed mRNA into a protein. The term “protein expression” on the other hand shall refer to the complete cellular process of synthesis of proteins. In one preferred example, an inhibiting modulator of the invention, such as an anti-sense molecule, may bind to the OR10H1 gene or mRNA and reduce transcription and/or translation or the OR10H1 mRNA. The terms “inhibitor of expression of a variant of OR10H1” and the like, shall have the corresponding meaning with respect to a variant of OR10H1.

The terms “inhibitor of OR10H1 stability” and the like (including similarly, “antagonist of OR10H1 stability” and the like) shall refer to any of the herein disclosed modulators (for example, the antigen binding constructs or anti-sense molecules described herein), which has a negative activity towards the stability of an OR10H1 protein. The term, in context of the present disclosure, shall be understood in its broadest sense. Such modulators are included by the term, which, for example, interfere with and reduce the OR10H1 protein half-live or interfere with and disturb OR10H1 protein folding or protein presentation on the surface of the cell. In one preferred example, an inhibiting modulator of the invention, such as an antigen binding construct, may induce internalisation, and optionally degradation, of OR10H1 protein from the surface of the cell. Other inhibiting modulators are included by the term, which, for example, interfere with and reduce the OR10H1 mRNA half-live or interfere with and disturb OR10H1 mRNA presence in the cytoplasm of the cell or presentation to a ribozyme. The terms “inhibitor of stability of a variant of OR10H1” and the like, shall have the corresponding meaning with respect to a variant of OR10H1.

The terms an “inhibitor of OR10H1 function” and the like (including similarly, “antagonist of OR10H1 function” and the like) shall refer to any of the herein disclosed modulators (for example, the antigen binding constructs or anti-sense molecules described herein) that impairs, such as induces a decrease or reduction in the amount or rate of one or more activities of OR10H1 protein or mRNA (for example, by impairing the expression and/or stability of OR10H1 protein or mRNA), such as one or more of those activities described herein, for example, the activity of OR10H1 as a modulator of T-cell activation and/or viability. For example, such an inhibiting modulator may impair binding of one or more of the chemostimuli of OR10H1 protein, may impair signaling by the G protein-coupled adenylyl cyclase cascade of OR10H1 protein and/or may impair the coupling or activity of one or more of the Gαs and Gα15 G proteins of OR10H1 protein. The terms “inhibitor of function of a variant of OR10H1” and the like, shall have the corresponding meaning with respect to a variant of OR10H1.

Such an inhibiting modulator can act directly, for example, by binding to OR10H1 and decreasing the amount or rate of one or more of the properties of OR10H1 such as its expression, function and/or stability. An OR10H1 antagonist or inhibitor can also decrease the amount or rate of OR10H1 function or activity by impairing its expression, stability, for example, by binding to OR10H1 protein or mRNA and modifying it, such as by removal or addition of a moiety, or altering its three-dimensional conformation; and by binding to OR10H1 protein or mRNA and reducing its stability or conformational integrity. An OR10H1 antagonist or inhibitor can also act indirectly, for example, by binding to a regulatory molecule or gene region to modulate regulatory protein or gene region function and affect a decrease in the amount or rate of OR10H1 expression, function and/or stability, in particular by impairing one or more activity of OR10H1 protein or mRNA (such as by changing the amount or rate of expression and/or stability of OR10H1 protein or mRNA). Thus, an OR10H1 inhibitor or antagonist can act by any mechanisms that impair, such as result in a decrease in, the amount or rate of OR10H1 expression, function and/or stability.

Therefore, a “functional variant” of OR10H1 (such as a functional fragment of an OR10H1 protein) is a variant of, such as a fragment of, the protein of OR10H1 that provides, possesses and/or maintains one or more of the herein described functions/activities of the non-variant protein of OR10H1. For example, such functional variant may bind one or more of the same chemostimuli as OR10H1 protein, may signal the same G protein-coupled adenylyl cyclase cascade as the OR10H1 protein and/or may be coupled to one or more of the same Gαs and Gα15 G proteins as OR10H1 protein, such as having the same, essentially the same or similar specificity and/or function as a receptor as OR10H1 protein. In other embodiments, such a functional variant may possess other activities than those possessed by the non-variant OR10H1 protein, as long as, preferably, it provides, possesses and/or maintains at least one function/activity that is the same, essentially the same or similar as OR10H1 protein. In more preferred embodiments, a functional variant of OR10H1 protein may act as an immune checkpoint inhibitor, such as by inhibiting cell-based immune response to a cancer cell that expresses such functional variant.

Antigen Binding Construct

In an additional aspect, the present invention pertains to, and preferred embodiments of other aspects of the invention involve, an antigen binding construct, capable of specifically binding to OR10H1, or to a variant of OR10H1, optionally wherein the antigen binding construct inhibits the expression, function and/or stability of OR10H1, or the variant of OR10H1. In particular embodiments, the antigen binding construct of the invention binds to protein of OR10H1, or of the variant of OR10H1, under conditions that are similar, essentially the same or the same as physiological conditions, such as those which can be created or mimicked by one or more of the in-vitro assays described by the examples herein.

The term “antigen binding construct” includes all varieties of antibodies and T cell receptor (TCR) derived polypeptides, which comprise an epitope binding domain, including binding fragments thereof. Further included are constructs that include 1, 2, 3, 4, 5, and/or 6 Complementary Determining Region (CDR)s, the main regions mediating antibody or TCR binding ability and specificity to a given antigenic epitope. In some embodiments, these CDRs can be distributed between their appropriate framework regions in a typical antibody or TCR variable domain. In some embodiments, the CDRs can be within a single peptide chain in others they are located in two or more peptide chains (heavy/light or alpha/beta respectively). In some embodiments, the two or more peptides are covalently linked together, for example via disulfide bonds. In some embodiments, they can be linked via a linking molecule or moiety. In some embodiments, the antigen binding proteins are non-covalent, such as a diabody and a monovalent scFv. Unless otherwise denoted herein, the antigen binding constructs described herein bind to an OR10H1 protein, or a variant protein of OR10H1 as described in detail herein above. Preferred embodiments of the invention pertain to antibodies, or antibody derived polypeptides, as antigen binding constructs of the invention.

In a more preferred embodiment, the antigen binding construct is isolated.

The term “isolated” as used herein in the context of a polypeptide, such as an antigen binding construct (an example of which could be an antibody), refers to a polypeptide that is purified from proteins or polypeptides or other contaminants that would interfere with its therapeutic, diagnostic, prophylactic, research or other use. An antigen binding construct according to the invention may be a recombinant, synthetic or modified (non-natural) antigen binding construct. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. In this context, a “recombinant” protein/polypeptide or nucleic acid is one made using recombinant techniques. Methods and techniques for the production of recombinant nucleic acids and proteins are well known in the art.

In a preferred embodiment, the (isolated) antigen binding construct of the invention comprises an antibody variable heavy and/or light chain sequence of an antibody obtainable from Di-8A11-H12-E6 (DSMZ Deposition Number: DSM ACC3310, deposited 26 Oct. 2016). In another embodiment, the isolated antigen binding construct is an antibody obtainable from hybridoma Di-8A11-H12-E6 (DSMZ Deposition Number: DSM ACC3310, deposited 26 Oct. 2016), or it is an antigen binding fragment obtainable from such antibody. Hybridoma clone Di-8A11-H12-E6 is a hybridoma comprising Rat B lymphocytes fused with murine Ag8 myeloma cells, and can be cultured at 37° C. in a 5% CO₂ atmosphere in RPMI-1640 medium supplemented with 10% Fetal calf serum, 10 U/ml human recombinant IL-6 and 1% penicillin-streptomycin (10,000 U/mL). The optimal split ratio is 1:1. The viability of these hybridoma cells may be relatively low when first thawed, however this recovers over a few days in culture.

In particular embodiments, such hybridoma-obtainable antigen binding construct is capable of specifically binding to OR10H1, or to a variant of OR10H1, and in more particularly embodiments wherein such antigen binding construct inhibits the expression, function and/or stability of OR10H1, or the variant of OR10H1.

The invention furthermore provides, and preferred embodiments of other aspects of the invention involve, an (isolated) antigen binding construct comprising at least one CDR3 having an amino acid sequence with at least 80% sequence identity to an amino acid sequence selected from SEQ ID Nos: 3, 7, 11, 15, 19, and 23. Alternatively, the antigen binding construct of the invention comprises a CDR3 region that has an amino acid sequence having no more than three or two, preferably no more than one amino acid substitution(s), deletion(s) and/or insertion(s) relative to a sequence selected from the group consisting of SEQ ID NOs. 3, 7, 11, 15, 19, and 23. Some CDR3 sequences of the invention are 8 amino acids long (see table D1 below). For such longer CDR3 sequences, the degree of sequence identity may be preferably increased in some embodiments to 85%. In particular embodiments, such CDR3-comprising antigen binding construct is capable of specifically binding to OR10H1, or to a variant of OR10H1, and in more particularly embodiments wherein such antigen binding construct inhibits the expression, function and/or stability of OR10H1, or the variant of OR10H1.

In some additional embodiments, the antigen binding construct further comprises at least one CDR1, and at least one CDR2. Further preferred antigen binding constructs of the invention include at least two of each kind of CDR sequences.

As mentioned earlier, preferred antigen binding constructs are antibodies and antibody-like constructs. The term “antibody” includes, but is not limited to, genetically engineered or otherwise modified forms of immunoglobulins, such as intrabodies, chimeric antibodies, fully human antibodies, humanized antibodies (e.g. generated by “CDR-grafting”), antibody fragments, and heteroconjugate antibodies (e.g., bispecific antibodies, diabodies, triabodies, tetra-bodies, etc.). The term “antibody” includes cys-diabodies and minibodies. Thus, each and every embodiment provided herein in regard to “antibodies”, or “antibody like constructs” is also envisioned as, bi-specific antibodies, diabodies, scFv fragments, chimeric antibody receptor (CAR) constructs, diabody and/or minibody embodiments, unless explicitly denoted otherwise. The term “antibody” includes a polypeptide of the immunoglobulin family or a polypeptide comprising fragments of an immunoglobulin that is capable of non-covalently, reversibly, and in a specific manner binding a corresponding antigen, preferably OR10H1, or a variant of OR10H1, as disclosed herein. An exemplary antibody structural unit comprises a tetramer. In some embodiments, a full length antibody can be composed of two identical pairs of polypeptide chains, each pair having one “light” and one “heavy” chain (connected through a disulfide bond). Antibody structure and isotypes are well known to the skilled artisan (for example from Janeway's Immunobiology, 9^{th} edition, 2016).

The recognized immunoglobulin genes of mammals include the kappa, lambda, alpha, gamma, delta, epsilon, and mu constant region genes, as well as the myriad immunoglobulin variable region genes (for more information on immunoglobulin genes see the international ImMunoGeneTics information system®, Lefranc M-P et al, Nucleic Acids Res. 2015 January; 43(Database issue):D413-22; and http://www.imgt.org/). For full-length chains, the light chains are classified as either kappa or lambda. For full-length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this invention, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)2) with the same, essentially the same or similar binding specificity. In some embodiments, the antibody binds specifically to protein of OR10H1, or of a variant of OR10H1. Preferred antigen binding constructs according to the invention include an antibody heavy chain, preferably the variable domain thereof, or an antigen binding fragment thereof, and/or an antibody light chain, preferably the variable domain thereof, or an antigen binding fragment thereof. In more preferred embodiments of the invention, the antigen binding fragment binds (such as specifically) to protein of OR10H1, or of a variant of OR10H1, and in most preferred embodiments wherein such antigen binding fragment inhibits the expression, function and/or stability of OR10H1, or the variant of OR10H1.

In some embodiments of the invention, the (isolated) antigen binding construct comprises the sequences of an antibody heavy chain variable region CDR1, CDR2, and CDR3; and/or the sequences of an antibody light chain variable region CDR1, CDR2, and CDR3. In preferred embodiments the antigen binding constructs of the invention comprise an antibody heavy chain sequence and/or an antibody light chain sequence, or an antigen binding fragment thereof; wherein the antibody heavy chain sequence, or the fragment thereof, comprises a CDR3 having at least 80% (for 8 amino acid long CDR3 sequences preferably 85%) sequence identity to an amino acid sequence selected from SEQ ID Nos. 3, 11, and 19, and/or wherein antibody light chain sequence, or the fragment thereof, comprises a CDR3 having at least 80% (for 8 amino acid long CDR3 sequences preferably 85%) sequence identity to an amino acid sequence selected from SEQ ID Nos. 7, 15, and 23.

In some embodiments the antibody heavy chain sequence of an antigen binding construct of the invention, or the antigen binding fragment thereof, further comprises a CDR1 having at least 80% sequence identity to an amino acid sequence selected from SEQ ID Nos. 1, 9, and 17; and/or a CDR2 having at 80% sequence identity to an amino acid sequence selected from SEQ ID Nos. 2, 10, and 18. In some embodiments the antibody light chain sequence, or the antigen binding fragment thereof, further comprises a CDR1 having at least 80% sequence identity to an amino acid sequence selected from SEQ ID Nos. 5, 13, and 21; and/or a CDR2 having at least 80% sequence identity to an amino acid sequence selected from SEQ ID Nos. 6, 14, and 22. Also in case of CDR1 or CDR2 sequences, the preferred degree of identity may be increased depending on the length of the CDR sequence. Preferred higher degrees of sequence identity include 85%, 90%, and 95%.

In some embodiments the (isolated) antigen binding construct according to the invention comprises an antibody variable chain sequence having at least 80%, preferably 85%, 90%, 95%, or 98% sequence identity to an amino acid sequence selected from SEQ ID Nos. 4, 8, 12, 16, 20, and 24.

In some embodiments the (isolated) antigen binding construct of the invention comprises an antigen binding fragment of an antibody, wherein said antigen binding fragment comprises CDR1, CDR2 and CDR3, preferably which are selected from the CDR1, CDR2 and CDR3 sequences having the respective amino acid sequences of SEQ ID Nos. 1, 2, 3; or 5, 6, 7; or 9, 10, 11; or 13, 14, 15; or 17, 18, 19; or 21, 22, 23; in each case independently, optionally with not more than three or two, preferably no more than one, amino acid substitution(s), insertion(s) and/or deletion(s) compared to these sequences.

In some embodiments of the invention, said CDR1 has an amino acid sequence of SEQ ID No 1, 5, 9, 13, 17 or 21, and CDR2 has an amino acid sequence of SEQ ID No 2, 6, 10, 14, 18, or 22, and CDR3 has an amino acid sequence of SEQ ID No 3, 7, 11, 15, 19, and 23; in each case independently, optionally with not more than three or two, preferably no more than one, amino acid substitution(s), insertion(s) and/or deletion(s) compared to these sequences.

In alternative or additional embodiments of the invention, the (isolated) antigen binding construct is an antibody, or an antigen binding fragment thereof, composed of at least one, preferably two, antibody heavy chain sequences, and at least one, preferably two, antibody light chain sequences, wherein at least one, preferably both, of said antibody heavy chain sequences comprise CDR1 to CDR3 sequences having the amino acid sequences of SEQ ID NO: 1 to 3, and at least one, preferably both, of said antibody light chain sequences comprise CDR1 to CDR3 sequences having the amino acid sequences of SEQ ID NO: 5 to 7; or wherein at least one, preferably both, of said antibody heavy chain sequences comprise CDR1 to CDR3 sequences having the amino acid sequences of SEQ ID NO: 9 to 11, and at least one, preferably both, of said antibody light chain sequences comprise CDR1 to CDR3 sequences having the amino acid sequences of SEQ ID NO: 13 to 15; or wherein at least one, preferably both, of said antibody heavy chain sequences comprise CDR1 to CDR3 sequences having the amino acid sequences of SEQ ID NO: 17 to 19, and at least one, preferably both, of said antibody light chain sequences comprise CDR1 to CDR3 sequences having the amino acid sequences of SEQ ID NO: 21 to 23; in each case of a CDR independently, optionally with not more than three or two, preferably no more than one, amino acid substitution(s), insertion(s) or deletion(s) compared to these sequence.

In alternative or additional embodiments of the invention, an (isolated) antigen binding construct is preferred, wherein the antigen binding construct is an antibody, or an antigen binding fragment thereof, composed of at least one, preferably two, antibody heavy chain sequence, and at least one, preferably two, antibody light chain sequence, wherein said antibody heavy chain sequence comprises a variable region having the amino acid sequence of SEQ ID NO: 4, and wherein said antibody light chain sequence comprises a variable region sequence having the amino acid sequence of SEQ ID NO: 8; or wherein said antibody heavy chain sequence comprises a variable region sequence having the amino acid sequence of SEQ ID NO: 12, and wherein said antibody light chain sequence comprises a variable region sequence having the amino acid sequence of SEQ ID NO: 16; or wherein said antibody heavy chain sequence comprises a variable region sequence having the amino acid sequence of SEQ ID NO: 20, and wherein said antibody light chain sequence comprises a variable region sequence having the amino acid sequence of SEQ ID NO: 24; in each case of a variable region sequence independently, optionally with not more than ten, nine, eight, seven, six, five, four, three, two or one, preferably not more than three, amino acid substitutions, insertions or deletions compared to these sequence.

In an additional aspect, the present invention pertains to, and preferred embodiments of other aspects of the invention involve, an (isolated) antigen binding construct that competes for antigen binding to an antigen binding construct comprising at least one Complementary Determining Region (CDR) 3 having an amino acid sequence with at least 80% sequence identity to an amino acid sequence selected from SEQ ID NOs. 3, 7, 11, 15, 19, and 23. In particular embodiments, such CDR3-comprising antigen binding construct is capable of specifically binding to OR10H1, or to a variant of OR10H1, and in more particular embodiments wherein such antigen binding construct inhibits the expression, function and/or stability of OR10H1, or the variant of OR10H1. In a preferred embodiment, such an antigen binding construct competes for binding (such as to OR10H1 protein, or to protein of a variant of OR10H1) with one or more of the antibodies of the invention described in the example, such competing for binding with 1C3-A1-A1, 1C3-A1-A2 and/or 8A11-B9-A1. In a more preferred embodiment, such as an antigen binding construct competes for binding (such as to OR10H1 protein, or to protein of a variant of OR10H1) with the antibody 8A11-H12-E6; and/or with an antibody obtainable from hybridoma Di-8A11-H12-E6 (DSMZ Deposition Number: DSM ACC3310, deposited 26Oct. 2016).

The term “compete” when used in the context of antigen binding constructs (e.g., modulating antigen binding constructs) that compete for the same epitope, means competition between antigen binding constructs as determined by an assay in which the antigen binding construct (e.g., antibody or antigen binding fragment thereof) being tested prevents or inhibits (e.g., reduces) binding of a reference antigen binding fragment (e.g., a reference antibody such as one described in the examples like 8A11-H12-E6/one obtainable from hybridoma Di-8A11-H12-E6, DSMZ Deposition Number: DSM ACC3310, deposited 26 Oct. 2016) to a common antigen (e.g., OR10H1 protein or a fragment thereof). Numerous types of competitive binding assays can be used to determine if one antigen binding construct competes with another, for example: solid phase direct or indirect radioimmunoassay (RIA), solid phase direct or indirect enzyme immunoassay (EIA), sandwich competition assay; solid phase direct biotin-avidin EIA etc. Typically, such an assay involves the use of purified antigen bound to a solid surface or cells bearing either of these, an unlabelled test antigen binding construct and a labelled reference antigen binding construct. Competitive inhibition is measured by determining the amount of label bound to the solid surface or cells in the presence of the test antigen binding construct. Usually the test antigen binding construct is present in excess. Antigen binding constructs identified by competition assay (competing antigen binding constructs) include antigen binding constructs binding to the same epitope as the reference antigen binding construct and antigen binding constructs binding to an adjacent epitope sufficiently proximal to the epitope bound by the reference antigen binding construct for steric hindrance to occur. Usually, when a competing antigen binding construct is present in excess, it will inhibit (e.g., reduce) specific binding of a reference antigen binding construct to a common antigen (such as OR10H1 protein or a fragment thereof) by at least 20%, 30%, 40%, 50%, 55%, 60%, 65%, 70%, 75% or more. In some instances, binding is inhibited by at least 80, 85%, 90%, 95%, or 97% or more, in particular between about 20% and 80%, preferably by at least about 30%, and more preferably by at least about 50%.

In preferred embodiments of the invention, the (isolated) antigen binding construct (such as one that is an inhibitor or antagonist of expression, function and/or stability of said OR10H1, or the variant of OR10H1) decreases or reduces the resistance of cells (such as tumour cells) that express OR10H1, or the variant of OR10H1, to an immune response. In other certain preferred embodiments of the invention, the (isolated) antigen binding construct (such as one that is an inhibitor or antagonist of expression, function and/or stability of said OR10H1, or the variant of OR10H1) enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a variant of OR10H1) that is mediated by cytotoxic T-cells and/or TILs. In other particular of such embodiments, the immune response is a cytotoxic immune response against cells (such as tumour cells) that express OR10H1 (or a variant of OR10H1), in particular a cell-mediated cytotoxic immune response such as one mediated by T-cells including cytotoxic T-cells and/or TILs.

Specifically, in certain preferred embodiments the (isolated) antigen binding construct (such as one that is an inhibitor or antagonist of expression, function and/or stability of said OR10H1, or the variant of OR10H1) enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a variant of OR10H1) are, in certain of such preferred embodiments, cancer cells or are cells that originated from a tumor cell. Exemplary cancer or tumor cells can be those as described or exemplified elsewhere herein.

In other certain preferred embodiments of the invention, the (isolated) antigen binding construct (such as one that is an inhibitor or antagonist of expression, function and/or stability of said OR10H1, or the variant of OR10H1) increases T-cell activity and/or survival, which in certain embodiments, may lead to an enhancement of a (cytotoxic) immune response mediated by such T-cells.

In some embodiments the (isolated) antigen binding construct of the invention may comprise in at least one, preferably all, polypeptide chains, antibody constant domain sequences. The origin of the constant domain sequence may be selected from a mouse, rat, donkey, rabbit or human antibody constant domain sequence. The selection of the constant domain is dependent on the intended use of the antigen binding construct of the invention. In some embodiments of the invention the antigen binding construct is chimerized, optionally is humanized or murinized. Preferred embodiments of the invention pertain to antigen binding constructs that comprise a rat heavy chain constant domain selected from constant domain sequences of rat IgG 1, 2a, 2b, 2c, and/or rat light chain constant domain kappa A allele or kappa B allele sequences (see Table E3 below). In preferred embodiments of those antibodies of the invention described in the examples, the IgG subclass of the heavy chain is a rat IgG2a or a rat IgG2b (see Table E3 below), and in more preferred of such embodiments, the IgG subclass of the heavy chain of antibody 1C3-A1-A1, 1C3-A1-A2 or 8A11-B9-A1 is rat IgG2b, and the IgG subclass of the heavy chain of antibody 8A11-H12-E6 is rat IgG2a (in each case, see Table E3).

A preferred embodiment of the invention pertains to a monoclonal antibody as an (isolated) antigen binding construct. An antibody of the invention may be an IgG type antibody, for example having any of the IgG isotypes.

A modulator of OR10H1 activity, or of the activity of a variant of OR10H1, that is an antibody can be, for example, an antibody that binds to OR10H1, or to the variant of OR10H1, and modulates, such as inhibits, OR10H1 function, or the function of the variant of OR10H1, or alters the activity of a molecule that regulates OR10H1, or the variant of OR10H1, expression or activity, such that the amount or rate of function of OR10H1, or of the variant of OR10H1, or its expression or stability is altered, such as decreased. An antibody useful in a method of the invention can be a naturally occurring antibody format, including monoclonal or polyclonal antibodies or fragments thereof, or a non-naturally occurring antibody format, including but not limited to a single chain antibody, chimeric antibody, bifunctional antibody, complementarity determining region-grafted (CDR-grafted) antibody, CAR, and humanized antibody or an antigen-binding fragment thereof.

In particular embodiments of the invention, the antigen binding construct, such as an antibody, is non-natural and/or is not a product of nature. In one of such embodiments, the antigen binding construct may be a non-natural antigen binding construct, such as a synthetic, modified or recombinant antigen binding construct. In particular, an antigen binding construct of the invention may contain at least one amino acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human antibody or a rabbit antibody (such as a polyclonal rabbit antibody) or a murine or rat antibody. In another of such embodiments, the antigen binding construct may be first generated following non-natural immunization of a (species of) mammal; such as by immunization with an antigen to which such (species of) mammal is not exposed in nature, and hence will not have naturally raised antibodies against.

Another aspect of the invention relates to a monoclonal antibody, or a binding fragment thereof, binding to and preferably modulating, more preferably inhibiting, OR10H1, or the variant of OR10H1. The present invention for the first time describes OR10H1 as a target for cancer treatment, and in particular as a target for modulating immune resistance of a cancer disease. Therefore, the present invention relates to the use of OR10H1 as a novel target for the generation of modulating, such as inhibitory, antibodies directed against the OR10H1 protein (or the protein of a variant of OR10H1). The generation of such antibodies is as such a standard procedure for the skilled artisan.

The anti-OR10H1 antibodies (or the anti-variant of OR10H1 antibodies) of the invention may be monoclonal or polyclonal antibodies. Monoclonal antibodies may be prepared using hybridoma-based methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

An immunizing agent typically includes the OR10H1, or the variant of OR10H1, protein, or fragments thereof, or a fusion protein thereof. However, antibodies may be prepared by genetic immunization methods in which native proteins are expressed in vivo with normal post-transcriptional modifications, avoiding antigen isolation or synthesis. For example, hydrodynamic tail or limb vein delivery of naked plasmid DNA expression vectors (eg, those encoding protein of OR10H1 or of a variant of OR10H1) can be used to produce the antigen of interest in vivo in mice, rats, and rabbits and thereby induce antigen-specific antibodies (Tang et al, Nature 356(6365): 152-4 (1992); Tighe et al, Immunol. Today 19(2) 89-97 (1998); Bates et al, Biotechniques, 40(2) 199-208 (2006); Aldevron-Genovac, Freiburg DE). This allows the efficient generation of high-titre, antigen-specific antibodies which may be particularly useful for diagnostic and/or research purposes. A variety of gene delivery methods can be used, including direct injection of naked plasmid DNA into skeletal muscle, lymph nodes, or the dermis, electroporation, ballistic (gene gun) delivery, and viral vector delivery.

Generally, either peripheral blood lymphocytes (“PBLs”) from the immunized host animal are isolated and used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding (1986) Monoclonal Antibodies: Principles and Practice, Academic Press, pp. 59-103). Immortalized cell lines may be transformed mammalian cells, particularly myeloma cells of rodent, bovine, and human origin. Rat- or mouse-myeloma cell lines may be employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor (1984) J. Immunol. 133:3001; Brodeuretal (1987) Monoclonal Antibody Production Techniques and Applications, Marcel Dekker, Inc., New York, pp. 51-631).

The culture medium in which the hybridoma cells are cultured can then be assayed for the presence of monoclonal antibodies directed against OR10H1 protein (or against protein of the variant of OR10H1). The binding specificity of monoclonal antibodies produced by the hybridoma cells can be determined by inmunoprecipitation or by an in vitro binding assay, such as radioimmunoassay (RIA) or enzyme-linked immunosorbent assay (ELISA). Such techniques and assays are known in the art. The binding affinity of the monoclonal antibody can be determined, for example, by the Scatchard analysis of Munson and Pollard (1980) Anal. Biochem. 107:220. Furthermore, in order to identify antibodies that modulate (for example, inhibit) the expression, function and/or stability of OR10H1 or of a variant of OR10H1, the candidate antibodies can be used in the herein described melanoma, CRC or pancreatic tumor TIL screening setup (see example section), or the herein described screening method of the invention. In particular, such antibodies are selected which increase the tumor cell susceptibility to TILs.

After the desired hybridoma cells are identified, the clones may be subcloned by limiting dilution procedures and grown by standard methods. The monoclonal antibodies secreted by the subclones may be isolated or purified from the culture medium or ascites fluid by conventional immunoglobulin purification procedures, such as, protein A-Sepharose, hydroxylapatite chromatography, gel electrophoresis, dialysis, or affinity chromatography.

Monoclonal antibodies of the present invention may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. The DNA also may be modified, for example, by substituting the coding sequence for human heavy and light chain constant domains in place of the homologous murine sequences (U.S. Pat. No. 4,816,567; Morrison et al., Proc Natl Acad Sci USA. 1984 November; 81(21): 6851-6855.) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

An antibody of the present invention may be a mouse, rat, rabbit, horse, goat, antibody, or a humanized or chimeric antibody. Most preferably, the antibody of the invention has a modulating effect, such as an inhibitory effect, on the immune modulatory function of OR10H1 (or of a variant of OR10H1) as described in context of the herein disclosed invention.

Another embodiment of the invention pertains to a hybridoma cell capable of producing an antigen binding construct, preferably a monoclonal or polyclonal antibody, as described above.

Yet a further aspect of the invention describes a method for producing an antibody against OR10H1, comprising a step of culturing a hybridoma cell of the invention and optionally recovering/isolating the antibody from the culture.

Yet, a further aspect of the invention provides a nucleic acid encoding for an antigen binding construct as described above. In a preferred embodiment, a nucleic acid of the invention is isolated.

In some embodiments, the polypeptides of the antigen binding constructs can be encoded by nucleic acids and expressed in vivo or in vitro. Thus, in some embodiments, a nucleic acid encoding an antigen binding construct is provided. In some embodiments, the nucleic acid encodes one part or monomer of an antigen binding construct of the invention (for example one of two chains of an antibody), and/or another nucleic acid encodes another part or monomer of an antigen binding construct of the invention (for example the other of two chains of an antibody). In some embodiments, the nucleic acid encodes two or more antigen binding construct polypeptide chains, for example, at least 2 antibody chains. Nucleic acids encoding multiple antigen binding construct chains can include nucleic acid cleavage sites between at least two chain sequences, can encode transcription or translation start site between two or more chains sequences, and/or can encode proteolytic target sites between two or more antigen binding construct chains.

Yet, one further aspect of the invention provides a vector (such as an expression vector) that comprises a nucleic acid encoding an antigen binding construct as disclosed herein, or a part or monomer of an antigen binding construct. For example, in some embodiments, where the antigen binding construct is a multimeric protein, the nucleic acid encodes only a single polypeptide chain of the antigen construct. Therefore, to express such an antigen binding construct, an expression vector of the invention may contain two or more nucleic acids that each encode a separate part or monomer of an antigen binding construct, which in combination would express an entire antigen binding construct. Analogously, an expression vector of the invention that comprises a nucleic acid that encodes only part or monomer of an antigen binding construct, may be used in combination with other separate expression vectors of the invention that each encode a separate part or monomer of an antigen binding construct. In other embodiments the nucleic acid encodes multiple polypeptide chains of the antigen binding construct of the invention. In some embodiments, the expression vector includes pcDNA3.1™/myc-His (−) Version A vector for mammalian expression (Invitrogen, Inc.) or a variant thereof. The pcDNA3.1 expression vector features a CMV promoter for mammalian expression and both mammalian (Neomycin) and bacterial (Ampicillin) selection markers. In some embodiments, the expression vector includes a plasmid. In some embodiments, the vector includes a viral vector, for example a retroviral or adenoviral vector. In embodiments, the vector includes a cosmid, YAC, or BAC.

Yet, another further aspect of the invention provides a cell line (such as a recombinant cell) which comprises a modulator of the present invention (as described herein, such as an antigen binding construct or antisense molecule of the invention) or at least one nucleic acid or at least one vector according to the invention. Preferably the cell line expresses at least one of the antigen binding constructs described herein. In some embodiments, a mammalian cell line (for example, a CHO cell line) is an expression system to produce the antigen binding constructs as described herein. In some embodiments, the antigen binding constructs or fragments thereof described herein are non-glycosylated, and a mammalian expression system is not required, as such, post-translational modifications are not needed. Thus, in some embodiments, one or more of a wide variety of mammalian or non-mammalian expression systems are used to produce the antigen binding constructs disclosed herein including, but not limited to mammalian expression systems (for example, CHO-K1 cells), bacterial expression systems (for example, E. Coli, B. subtilis) yeast expression systems (for example, Pichia, S. cerevisiae) or any other known expression system. Other systems can include insect cells and/or plant cells.

Furthermore provided is a recombinant host cell, comprising a modulator of the present invention (as described herein, such as an antigen binding construct or antisense molecule of the invention), or at least one nucleic acid, or at least one vector according to the invention. The recombinant host cell in some embodiments is preferably a human cell, preferably an autologous human cell. More preferably, the recombinant host cell is cable of expressing an antigen binding construct of the invention.

Another aspect of the invention then pertains to a method for producing (such as manufacturing) a recombinant cell line capable of expressing an antigen binding construct of the invention, such as one specific for OR10H1, or for a OR10H1 variant, comprising
(a) providing a suitable host cell,
(b) providing a at least one genetic construct comprising one or more coding sequence that (eg, in combination) encode the antigen binding construct as described herein,
(c) introducing into said suitable host cell said genetic construct(s),
(d) optionally, expressing said genetic construct(s) by said suitable host cell under conditions that allow for the expression of the antigen binding construct, and optionally conditions that allow for assembly of the antigen binding construct.

One further aspect of the invention then pertains to a method for producing (such as manufacturing) an antigen binding construct of the invention, such as one specific for OR10H1, or for a OR10H1 variant, said method comprising
(a) providing a hybridoma or host cell capable of expressing an antigen binding construct of the invention, for example a recombinant cell line comprising at least one genetic construct comprising one or more coding sequence(s) that (eg, in combination) encode an antigen binding construct of the invention,
(b) culturing said hybridoma or host cell under conditions that allow for the expression of the antigen binding construct, and optionally conditions that allow for assembly of the antigen binding construct.

In some embodiments, either of these methods may further include the isolation and/or purification of said expressed and optionally assembled antigen binding construct from the host cell. The genetic construct(s) may be an expression construct comprising a promoter sequence operably linked to said coding sequence. In some embodiments, said antigen binding construct is of mammalian origin, preferably of human origin. In some embodiments said suitable host cell is a mammalian cell, optionally a CHO cell. In some further embodiments, said antigen binding construct is a modified antibody, wherein said modification comprises addition of a functional moiety selected from a detectable label or a cytotoxic moiety.

Antisense Molecules

A modulator of, such as an inhibitor or antagonist of, expression, function and/or stability of OR10H1, or of a variant of OR10H1, that is a nucleic acid can be, for example, an anti-sense nucleotide molecule, an RNA, DNA or PNA molecule, or an aptamer molecule. An anti-sense nucleotide molecule can, by virtue of it comprising an anti-sense nucleotide sequence, bind to a target nucleic acid molecule (eg based on sequence complementarity) within a cell and modulate the level of expression (transcription and/or translation) of OR10H1 (or of a variant of OR10H1), or it may modulate expression of another gene that controls the expression, function and/or stability of OR10H1 (or the variant). Similarly, an RNA molecule, such as a catalytic ribozyme, can bind to and alter the expression of the OR10H1 gene, (or of the variant of OR10H1 gene), or it can bind to and alter the expression of other genes that control the expression, function and/or stability of OR10H1 (or the variant), such as a transcription factor for or repressor protein of OR10H1 (or for the variant). An aptamer is a nucleic acid molecule that has a sequence that confers it an ability to form a three-dimensional structure capable of binding to a molecular target.

A modulator of expression, function and/or stability of OR10H1, or of a variant of OR10H1, that is a nucleic acid also can be a double-stranded RNA molecule for use in RNA interference. RNA interference (RNAi) is a process of sequence-specific gene silencing by post-transcriptional RNA degradation or silencing (prevention of translation). RNAi is initiated by use of double-stranded RNA (dsRNA) that is homologous in sequence to the target gene to be silenced. A suitable double-stranded RNA (dsRNA) for RNAi contains sense and antisense strands of about 21 contiguous nucleotides corresponding to the gene to be targeted that form 19 RNA base pairs, leaving overhangs of two nucleotides at each 3′ end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. USA 98:7863-7868 (2001). dsRNA can be synthesized in vitro and introduced into a cell by methods known in the art.

A particularly preferred example of an antisense molecule of the invention is a small interfering RNA (siRNA) or endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesized siRNA for RNA Interference (RNAi). An esiRNAs is the enzymatic digestion of a long double stranded RNA in vitro.

As described above, a modulator of the invention that is an RNAi molecule (such as an siRNA) may bind to and directly inhibit or antagonise the expression of mRNA of OR10H1 (or of a variant of OR10H1). However, a modulator of the invention that is an RNAi molecule (such as an siRNA) may bind to and inhibit or antagonise the expression of mRNA of another gene that itself controls the expression (or function or stability) of OR10H1, or of the variant. Such other genes may include transcription factors or repressor proteins.

The sequence identity of the antisense molecule according to the invention in order to target a OR10H1 mRNA, or an mRNA of a OR10H1 variant protein (or to target mRNA of a gene controlling expression, function and/or stability of OR10H1 or the variant), is with increasing preference at least 75%, at least 80%, at least 85%, at least 90%, at least 95%, at least 98%, at least 99% and 100% identity to a region of a sequence encoding the OR10H1 protein, or variant thereof, as disclosed herein (or of such other controlling gene). Preferably, the region of sequence identity between the target gene and the modulating antisense molecule is the region of the target gene corresponding to the location and length of the modulating antisense molecule. For example, such a sequence identity over a region of about 19 to 2lbp of length corresponding to the modulating siRNA or shRNA molecule). Software, tools, and/or (other) means and methods for determining sequence identity are known in the art. Preferably, the BLAST (Basic Local Alignment Search Tool) program is used for determining the sequence identity with regard to one or more OR10H1 RNAs as known in the art. On the other hand, preferred antisense molecules such as siRNAs and shRNAs of the present invention are preferably chemically synthesized using appropriately protected ribonucleoside phosphoramidites and a conventional RNA synthesizer. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

The ability of antisense molecules, siRNA, and shRNA to potently, but reversibly, silence genes in vivo makes these molecules particularly well suited for use in the pharmaceutical composition of the invention which will be also described herein below. Ways of administering siRNA to humans are described in De Fougerolles et al., Current Opinion in Pharmacology, 2008, 8:280-285. Such ways are also suitable for administering other small RNA molecules like shRNA. Accordingly, such pharmaceutical compositions may be administered directly formulated as a saline, via liposome based and polymer-based nanoparticle approaches, as conjugated or complexation pharmaceutical compositions, or via viral delivery systems. Direct administration comprises injection into tissue, intranasal and intratracheal administration. Liposome based and polymer-based nanoparticle approaches comprise the cationic lipid Genzyme Lipid (GL) 67, cationic liposomes, chitosan nanoparticles and cationic cell penetrating peptides (CPPs). Conjugated or complexation pharmaceutical compositions comprise PEI-complexed antisense molecules, siRNA, shRNA or miRNA. Further, viral delivery systems comprise influenza virus envelopes and virosomes.

The antisense molecules, siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides. Particularly preferred example of siRNAs is GapmeR (LNA™ GapmeRs (Exiqon)). GapmeRs are potent antisense oligonucleotides used for highly efficient inhibition of OR10H1 mRNA, or of mRNA of a OR10H1 variant protein (or of mRNA of a gene controlling expression, function and/or stability of OR10H1 or the variant). GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus.

Preferred antisense molecules for targeting OR10H1, or OR10H1-variant, are antisense molecules or constructs having a sequence complementary to a region (such as one described above) of a nucleic acid sequence of an OR10H1 mRNA, or of an mRNA of a variant of OR10H1, preferably a sequence complementary to a region of a sequence encoding the amino acid sequence shown in SEQ ID NO: 25, more preferably, a sequence complementary to a region of between about 15 to 25 bp (such as between about 19 and 21 bp) of a sequence encoding the amino acid sequence shown in SEQ ID NO: 25. Most preferred is an antisense molecule comprising, or consisting essentially of, a sequence according to SEQ ID NO: 26-34; for example an siRNA having a sequence at least 90% identical to a sequence according to any of SEQ ID NO: 26 to 29; or an shRNA having a sequence at least 90% identical to a sequence according to any of SEQ ID NO: 30 to 34. In one other preferred embodiments, the modulating siRNA molecule comprises, or consists essentially of, a sequence identical to a sequence according to any of SEQ ID NO: 26 to 29, optionally with no more than three, two or one, most preferably no more than one, nucleotide substitution or deletion compared to such sequence. In another preferred embodiments, the modulating shRNA molecule comprises, or consists essentially of, a sequence identical to a sequence according to any of SEQ ID NO: 30 to 34, optionally with no more than three, two or one, most preferably no more than one, nucleotide substitution or deletion compared to such sequence.

In one embodiment the antisense molecules of the invention may be isolated. In another embodiment, the antisense molecules of the invention may be recombinant, synthetic and/or modified, or in any other way non-natural or not a product of nature. For example, a nucleic acid of the invention may contain at least one nucleic acid substitution (or deletion) modification such as between 1 and about 5 such modifications, preferably no more than 1, 2 or 3 such modifications) relative to a product of nature, such as a human nucleic acid. As described above, the antisense molecules of the invention may be modified by use of non-natural nucleotides, or may be conjugated to another chemical moiety. For example, such chemical moieties may be a heterologous nucleic acid conferring increased stability or cell/nucleus penetration or targeting, or may be a non-nucleic acid chemical moiety conferring such properties, of may be a label.

Certain preferred embodiments pertain to a genetic construct for gene editing that is used as an inhibitor of expression, function and/or stability of OR10H1, or of a variant of OR10H1, in context of the herein described invention. By using genome editing constructs it is possible to modulate the expression, stability and/or activity of OR10H1, or its variants. Genome editing approaches are well known in the art and may be easily applied when the respective target genomic sequences are known. Preferably, such approaches may be used in gene therapy using e.g. viral vectors, which specifically target tumor cells in accordance with the above descriptions.

In case of genome editing, DNA is inserted, replaced, or removed, from a genome using artificially engineered nucleases, or so called “molecular scissors”. The nucleases create specific double-stranded break (DSBs) at desired locations in the genome, and harness the cell's endogenous mechanisms to repair the induced break by natural processes of homologous recombination (HR) and non-homologous end-joining (NHEJ). For doing so, engineered nucleases such as zinc finger nucleases (ZFNs), Transcription Activator-Like Effector Nucleases (TALENs), the CRISPR/Cas system, and engineered meganuclease re-engineered homing endonucleases are routinely used for genome editing. According to another preferred embodiment, for genome editing approaches for modulating/inhibiting OR10H1, or a variant of OR10H1, the rare-cutting endonuclease is Cas9, Cpfl, TALEN, ZFN, or a homing endonuclease may be used. Also, it may be convenient to engineer using DNA-guided Argonaute interference systems (DAIS). Basically, said Argonaute (Ago) protein is heterologously expressed from a polynucleotide introduced into said cell in the presence of at least one exogenous oligonucleotide (DNA guide) providing specificity of cleavage to said Ago protein to a preselected locus. The TALEN and Cas9 systems are respectively described in WO 2013/176915 and WO 2014/191128. The Zinc-finger nucleases (ZFNs) are initially described in Kim, Y G; Cha, J.; Chandrasegaran, S. (“Hybrid restriction enzymes: zinc finger fusions to Fok I cleavage domain” (1996). Proc Natl Acad Sci USA 93 (3): 1156-60). Cpfl is class 2 CRISPR Cas System described by Zhang et al. (Cpfl is a single RNA-guided Endonuclease of a Class 2 CRIPR-Cas System (2015) Cell; 163:759-771). The argonaute (AGO) gene family was initially described in Guo S, Kemphues K J. (“par-1, a gene required for establishing polarity in C. elegans embryos, encodes a putative Ser/Thr kinase that is asymmetrically distributed” (1995) Cell;81(4):611-20).

The use of the CRISPR/Cas9, CRISPR/Cpfl or the Argonaute genome-editing systems is particularly adapted to be used in combination with the transfection of guide RNA or guide DNA sequences. In this context the guide-RNAs and a nucleic acid sequence coding for Cas9 nickase (or similar enzymes), is transfected into a target cell (preferably a tumor cell) so that they form a complex able to induce a nick event in double-stranded nucleic acid targets in order to cleave the genetic sequence between said nucleic acid targets.

In certain embodiments, it may be useful to deliver the guide RNA-nanoparticle formulations separately from the Cas9. In such an instance a dual-delivery system is provided such that the Cas 9 may be delivered via a vector and the guide RNA is provided in a nanoparticle formulation, where vectors are considered in the broadest sense simply as any system for and/or (other) means of delivery, rather than specifically viral vectors. Separate delivery of the guide RNA-nanoparticle formulation and the Cas 9 may be sequential, for example, first Cas9 vector is delivered via a vector system followed by delivery of sgRNA-nanoparticle formulation) or the sgRNA-nanoparticle formulation and Cas9 may be delivered substantially contemporaneously (i.e., co-delivery). Sequential delivery may be done at separate points in time, separated by days, weeks or even months. In certain embodiments, multiple guide RNAs formulated in one or more delivery vehicles (e.g., where some guide RNAs are provided in a vector and others are formulated in nanoparticles) may be provided with a Cas9 delivery system. In certain embodiments, the Cas9 is also delivered in a nanoparticle formulation. In such an instance the guide RNA-nanoparticle formulation and the Cas9 nanoparticle formulation may be delivered separately or may be delivered substantially contemporaneously (i.e., co-delivery).

As will now be apparent to the person of ordinary skill, the gene target of such genome-editing approaches may be the gene of OR10H1, or the gene of the variant of OR10H1. Alternatively, the gene target of such editing may be another gene that controls the expression, function and/or stability of OR10H1 or of the variant.

In preferred embodiments of the invention, the compounds for genome editing approaches according to the invention comprise at least the use of a guide RNA or DNA complementary to a region (such as one described above) of an OR10H1, or OR10H1 variant, sequence. In some additional embodiments, the compounds for use in genome editing approaches of the invention may include donor sequences homologous to such a region of OR10H1, or a variant of OR10H1, as templates for homology directed repair. The donor sequences comprise a mutated sequence of OR10H1, or of a variant of OR10H1, that when used in the CRISPR induced repair mechanism in a target cell, is by homologous recombination inserted/copied into the OR10H1 genomic locus, and therefore yields into a mutated OR10H1 gene which is characterized by a reduced expression, function and/or stability of the expressed OR10H1, or of the variant of OR10H1. CRISPR/Cas9 genome editing in cancer therapy is reviewed for example in Khan FA(1) et al: “CRISPR/Cas9 therapeutics: a cure for cancer and other genetic diseases.” (Oncotarget. 2016 May 26. doi: 10.18632/oncotarget.9646; incorporated by reference in its entirety).

In a preferred embodiment of the present invention, the compound is a nucleic acid inhibitor of expression (and/or stability) of OR10H1, or of a variant of OR10H1. For example in such embodiments, the compound can be an antisense nucleic acid (such as an antisense DNA, RNAi, siRNA, shRNA or guide nucleic acid) that binds to a nucleic acid that encodes (or regulates; such as a promoter or enhancer region) the expression of: (i) said OR10H1 or of the variant of OR10H1; or (ii) a gene that controls the expression (and/or stability) of said OR10H1 or of the variant of OR10H1, where such antisense nucleic acid inhibits the expression (and/or stability) of said OR10H1, or of the variant of OR10H1.

Treatment Methods and Diseases

A method of treatment of the invention preferably, comprises a step of contacting a cell with a modulator of OR10H1, or variant of OR10H1, expression, stability and/or function, more preferably contacting a tumor or cancer cell with an inhibitor of OR10H1, or variant of OR10H1, expression, stability and/or function. In context of the invention, it was surprisingly found that OR10H1 mediates melanoma, pancreatic and colorectal tumor cell resistance against cytotoxic T lymphocytes (CTL) by inhibiting tumor infiltrating lymphocyte (TIL) viability and/or activity. Therefore, the present invention for the first time indicates a method for reducing resistance of a tumor or cancer cell to CTL responses by impairing OR10H1 expression, function and/or stability in the tumor. Thus, it is preferred that said tumor or cancer cell is characterized by a detectable cell surface expression of OR10H1 (or of a variant of OR10H1) before contacting the cell with an inhibitor of expression, function and/or stability of OR10H1, or of a variant of OR10H1. More preferred is that the tumor or cancer is characterized by a resistance of the tumor or cancer against autologous or heterologous T-cell mediated immune responses, and/or is a refractory/recurrent tumor or cancer, a metastatic tumor or a multidrug resistant tumor or cancer.

A disease treatable by the compounds and methods of the invention includes one or more proliferative diseases. “Proliferative disease” refers to a disease characterized by abnormal proliferation of cells. A proliferative disease does not imply any limitation with respect to the rate of cell growth, but merely indicates loss of normal controls that affect growth and cell division. Thus, in some embodiments, cells of a proliferative disease can have the same cell division rates as normal cells but do not respond to signals that limit such growth. Within the ambit of “proliferative disease” is neoplasm or tumor, which is an abnormal growth of tissue. Cancer refers to any of various malignant neoplasms characterized by the proliferation of cells that have the capability to invade surrounding tissue and/or metastasize to new colonization sites. Non-cancerous proliferative diseases also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

A disease treatable by the compounds and methods of the invention is preferably one characterised by a resistance of one or more cells affected by the disease (such as the proliferative disease) against a defence response of the host organism, such as resistance that is associated with a pathological phenotype. A preferred example is a resistance of a tumor or cancer against one or more immune responses, in particular cell-mediated immune response, mounted by the subject/patient suffering from the tumor or cancer. A disease treatable by the compounds and methods of the invention is more preferably one characterised by expression of OR10H1 (or expression of a variant of OR10H1); in particular one characterised by such expression that is aberrant, for example over- (or under-)expression on a given cell or tissue (such as those cells or tissues associated with the proliferative disease of the subject) compared to that in a healthy subject or a normal cell.

The term “resistance” refers to an acquired or natural resistance of a cell of a proliferative disease, such as tumor or cancer cell, to a patient's own immune response (such as a cell-mediated immune response), or to immune responses aided by immune therapy such as adoptive T-cell transfer. Therefore, a resistant tumor or cancer cell is more likely to escape and survive humoral and/or cellular immune defense mechanisms in a subject having the tumor or cancer. A treatment of a resistant proliferative disease, such as tumor/cancer resistance, in context of the invention shall be effective if compared to a non-treated control, the disease cell (such as a cell of the tumor of cancer) becomes more sensitive or susceptible to an immune response (such as a cell-mediated immune response)—in other words will be more likely to be “identified” and neutralized by a patient's immune response.

In preferred embodiments of the invention, the term “tumor resistance” is a resistance to a cell-mediated immune response, such as cytotoxic T lymphocyte (CTL) response (i.e., the tumor or tumor cell being nonresponsive to, or having reduced or limited response to a CTL targeting a tumor cell). A tumor cell may show a reduced sensitivity when contacted with a CTL specific for an antigen expressed on that tumor cell. A reduced sensitivity is a reduction to a 90% cytotoxic T cell response, preferably a reduction to 80%, 70%, 60%, 50% or more preferably a reduction to 40%, 30%, 20% or even less. In this case, 100% would denote the state wherein the CTLs can kill all of the cells in a cancer sample. Whether or not a tumor cell is resistant to a patient's (cell-mediated) immune response may be tested in-vitro by contacting autologous tumor cell with autologous T-cells and thereafter quantifying the survival/proliferation rate of the tumor cells. As an alternative, the reduction in (cell-mediated) immune response is determined by comparing cancer samples of the same cancer before and after the resistance is acquired (for example induced by therapy), or by comparing with a cancer sample derived from a different cancer which is known to have no resistance to the CTL.

On the other hand, the treatments of the present invention include the sensitization of tumor cells against CTL and therefor to decrease tumor cell resistance. A decrease of tumor cell resistance against CTL is preferably a significant increase of CTL toxicity, preferably a 10% increase, more preferably 20%, 30%, 40%, 50%, 60%, 70%, 80% or more, even more preferably 2 fold increase, 3 fold, 4 fold, 5 fold or more.

A proliferative disease in context of the invention may also be a tumor or cancer disease, selected from a liquid or solid tumor, and preferably is lung cancer, bladder cancer, ovarian cancer, uterine cancer, endometrial cancer, breast cancer, liver cancer, pancreatic cancer, stomach cancer, cervical cancer, lymphoma, leukemia, acute myeloid leukemia, acute lymphocytic leukemia, salivary gland cancer, bone cancer, brain cancer, colon cancer, rectal cancer, colorectal cancer, kidney cancer, skin cancer, melanoma, squamous cell carcinoma, pleomorphic adenoma, hepatocellular carcinoma, and/or adenocarcinoma. More preferably, the disease is one selected from the list consisting of: melanoma, pancreatic cancer and colorectal cancer. Most preferably, the disease is characterized by the expression of OR10H1, or of a variant of OR10H1, in a cell associated with the pathology of the disease. A preferred disease is in some embodiments an OR10H1- or OR10H1 variant protein-expressing tumor or cancer.

The compounds of the invention are preferably for use in aiding a cell-mediated immune response in a subject, such as aiding a patient's T-cell mediated immune response, against a proliferative diseases such as a tumor or cancer disease. For example, the compounds of the invention can be for use in a treatment that includes a transfer of cells such as a transfer of immune cells to the patient, in particular the compounds of the invention can be for use in a treatment that include adoptive T-cell transfer based therapy of cancer. The cells transferred in such treatment may include those being autologous cells of the subject, for example autologous immune cells, such as T-cells or Natural Killer (NK)-cells, of the subject.

As mentioned above, in certain embodiments the compound of the invention is an inhibitor or antagonist of expression, function and/or stability of OR10H1, or the variant protein thereof. In one particular of such embodiments, the inhibition of the expression, function and/or stability of said OR10H1, or the variant of OR10H1, enhances an immune response, preferably enhances a cell-mediated immune response in a subject (such as a patient), and more preferably enhances a T-cell mediated immune response in the subject. Such an enhancement of immune response can be used for treating a proliferative disease, for example treating a cancer disease. In another particular of such embodiments, the inhibition of the expression, function and/or stability of OR10H1, or the variant protein thereof, in the disease of a subject (such as the patient), such as a cancer, enhances an immune response (such a cell-mediated immune response) in the subject; in particular where such an enhancement may be associated with an increases T-cell activity and/or survival in said patient. In this context the T-cells may be endogenous T-cells, or transferred autologous or foreign T-cells.

It will now be apparent to the person of ordinary skill, given the present invention is related to the surprising finding of OR10H1-mediated resistance to one or more immune responses, that an “enhancement” or an “increase” in immune response (such as a cell-based immune response), in particular an increase in T-cell activity and/or survival, may be an increase or enhancement relative to that mediated (eg reduced) by this newly described OR10H1 activity (eg the resistance). Accordingly, the “enhancement” or “increase” (and like terms) may equally be considered a “release” of the reduction of an immune response mediated by OR10H1 (and reflected by the resistance to the immune response), such that the immune response resulting from inhibition of OR10H1 (or of the variant of OR10H1) expression, function and/or stability, may be so enhanced or increased relative to that in the subject prior to such inhibition, but the resulting immune response may also be considered to be at a level (after such OR10H1 inhibition) equivalent to that typically present in a healthy subject.

The increase in a subject's T-cell activity and/or T cell survival can, in certain embodiments of the invention, be associated with an inhibition of cAMP signaling mediated by OR10H1 or by the variant of OR10H1. In further embodiments, the increase in T-cell activity and/or survival may be associated with reduced phosphorylation of cAMP response element-binding protein (CREB); and/or with activation of lymphocyte-specific protein tyrosine kinase (Lck), for example by reduced phosphorylation of the LcK Tyr505 domain in such immune cell(s). In some of such embodiments, the increase in said T-cell activity and/or survival is associated with reduced phosphorylation of protein kinase A (PKA) in said immune T-cells. In further of such embodiments the increase in said T-cell activity and/or survival is associated with a reduction in the level of cAMP in said T-cells. The term “associated with”, in the context of such and other embodiments can mean that two variables, effects or phenotypes are correlated to each other, that they are related to each other, or that there is some kind of causative link between a first variable, effect or phenotype and the second such as the second is in response to the first, the second is a consequence of the first, or the second is caused by the first.

In certain preferred embodiments of the invention, the compound (such as a compound that is an inhibitor or antagonist of expression, function and/or stability of said OR10H1, or the variant of OR10H1) decreases or reduces the resistance of cells (such as tumour cells) that express OR10H1, or the variant of OR10H1, to an immune response. In other certain preferred embodiments of the invention, the compound (such as a compound that is an inhibitor or antagonist of expression, function and/or stability of said OR10H1, or the variant of OR10H1) enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a variant of OR10H1) that is mediated by cytotoxic T-cells and/or TILs. In other particular of such embodiments, the immune response is a cytotoxic immune response against cells (such as tumour cells) that express OR10H1 (or a variant of OR10H1), in particular a cell-mediated cytotoxic immune response such as one mediated by T-cells including cytotoxic T-cells and/or TILs.

Specifically, in certain preferred embodiments, the compound (such as a compound that is an inhibitor or antagonist of expression, function and/or stability of said OR10H1, or the variant of OR10H1) enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a variant of OR10H1) are, in certain of such preferred embodiments, cancer cells or are cells that originated from a tumor cell. Exemplary cancer or tumor cells can be those as described or exemplified elsewhere herein.

In other certain preferred embodiments of the invention, the compound (such as a compound that is an inhibitor or antagonist of expression, function and/or stability of said OR10H1, or the variant of OR10H1) increases T-cell activity and/or survival, which in certain embodiments, may lead to an enhancement of a (cytotoxic) immune response mediated by such T-cells.

Detection, Diagnosis and Screening

Another aspect of the invention further pertains to a method for detecting an OR10H1 protein, or a variant of OR10H1, in a (biological) sample—such as one obtained from a subject or patient—comprising contacting the sample with an antigen binding construct specifically binding to said OR10H1 protein, or to the variant of OR10H1, and detecting the binding between said antigen binding construct and said OR10H1 protein, or the variant of OR10H1. In some embodiments, the antigen binding construct is an antibody, or preferably the antigen binding construct according to the herein described invention. In some embodiments, the (biological) sample is a sample of a tumor or a cancer (such as one of those described elsewhere herein) for example a sample comprising tumor or cancer cells, preferably a tumor or cancer sample of a patient suffering from a refractory/recurrent tumor disease, a metastatic tumor disease, or a multidrug resistant tumor disease.

The object of the invention is furthermore solved by a method for diagnosing a resistance phenotype of a cancer disease, against an immune response in a patient, the method comprising the steps of
(a) Providing a sample of tumor or cancer cells of the subject,
(b) Determining the presence or absence of protein or mRNA of OR10H1, or of a variant of OR10H1, in the tumor or cancer cells, and
(c) Diagnosing a resistance phenotype of the cancer disease against an immune response in the subject when protein or mRNA of said OR10H1, or of the variant of OR10H1, is present in the tumor or cancer cells.

The diagnostic methods of the invention are in preferred embodiments performed ex vivo/in vitro.

In certain embodiments, the phenotype to be diagnosed by the method of the invention is one of an increased resistance phenotype, and/or one that is a resistance to a cell-mediated immune response such as a T-cell immune response.

In further certain embodiments of the diagnostic methods the presence of an amount or level of said protein or mRNA of OR10H1, or of the variant of OR10H1, is determined in step (b) as a measured amount.

In preferred embodiments, the presence of an amount or level of said protein or mRNA of OR10H1, or of the variant of OR10H1, in the tumor cells in step (b) is a measured amount of said protein or mRNA of OR10H1, or of the variant of OR10H1, which if greater than: (i) a measured quantity of said protein or mRNA of OR10H1, or of the variant of OR10H1, in control cells; and/or (ii) a standard or cut-off value for said quantity of said protein or mRNA of OR10H1, or of the variant of OR10H1, then the diagnosis in step (c) is made.

In some embodiments step (b) of the diagnostic method of the invention involves contacting the sample with a detector and/or (other) means for the detection of protein or mRNA of OR10H1, or of a variant of OR10H1, wherein said detector and/or (other) means is preferably selected from: (i) an antigen binding construct specifically binding to said protein of OR10H1, or of the variant of OR10H1, and detecting the binding between said antigen binding construct and said protein of OR10H1, or of the variant of OR10H1; and/or (ii) a nucleic acid binding to said mRNA sequence of OR10H1, or of the variant of OR10H1, and detecting the binding between said nucleic acid and said mRNA of OR10H1, or of the variant of OR10H1. Preferably, the antigen binding construct is an antigen binding construct as described herein before.

In some embodiments, such detection and diagnostic methods may be a computer-implemented method, or one that is assisted or supported by a computer. In some embodiments, information reflecting the presence/absence or an amount of OR10H1, or of the variant thereof, in a sample is obtained by at least one processor, and/or information reflecting the presence/absence or an amount of the OR10H1 or variant thereof in a sample is provided in user readable format by another processor. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. In some embodiments, the computer-implemented method of detecting the presence or an amount of OR10H1 or of a variant thereof in a sample is provided as a kit.

Therefore, the present invention also provides a kit (such as a detection and/or diagnostic kit) comprising a detector and/or (other) means for the determination of the presence or absence of OR10H1 (or of a variant of OR10H1), such as in a cell associated with a proliferative disease such as a tumor or cancer cell. The diagnostic kit is suitable for diagnosing an absent or decreased immune susceptibility of a proliferative disease to an immune response, such as of a tumor or cancer towards a cell-mediate immune response (eg that mediated T-cells). The kit may preferably comprise specific and selective anti-OR10H1 antibodies as described herein before. Alternatively, the diagnostic kit may comprise nucleic acid primers and/or probes for detecting the expression of OR10H1 in a tumor cell. The kit of the invention may include other known detector and/or (other) means for detecting OR10H1 protein expression. The kit may further comprise instructions for use and/or with one or more additional components useful for said detection. Such instructions may consist of a printed manual or computer readable memory comprising such instructions, or may comprise instructions as to identify, obtain and/or use one or more other components to be used together with the kit. Such additional component may comprise one or more other item, component, reagent or other moiety or means useful for the use of the kit or practice of a detection method of the invention, including any such item, component, reagent or moiety or means disclosed herein useful for such practice. For example, the kit may further comprise reaction and/or binding buffers, labels, enzymatic substrates, secondary antibodies and control samples, materials or moieties etc.

In preferred embodiments of the kit or the detection/diagnostic methods, the detector and/or (other) means for the detection of protein or mRNA of OR10H1, or of a variant of OR10H1, is labeled, for example is coupled to a detectable label. The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary anti-bodies, metal binding domains, epitope tags, etc.).

In another aspect, the invention relates to a method of diagnosing and treating a proliferative disease, such as one characterised by (aberrant) expression of OR10H1, or of a variant of OR10H1 (such as a tumor or cancer disease) in a subject, such as a patient, comprising:
(a) Providing a (biological) sample from said subject, said sample comprising cells or tissue of said subject, or an extract of said cells or tissue; and
(b) Detecting whether OR10H1 or the variant is present in the biological sample;
(c) Diagnosing the subject with the disease depending on if the presence of OR10H1 or the variant in the biological sample is detected; and
(d) Administering an effective amount of an inhibitor of the expression, function and/or stability of OR10H1 (or of a variant of OR10H1) function to the diagnosed subject, preferably an inhibiting or antagonistic compound of the invention as described herein.

In another aspect of the invention, a method is provided for identifying a compound suitable for the treatment of a disease characterized by expression (such by aberrant expression) of OR10H1, or a variant thereof, the method comprising the steps of
(a) Providing a first cell expressing a protein or mRNA of OR10H1, or of a variant of OR10H1, and
(b) Providing a candidate compound, and
(c) Optionally, providing a second cell which is a cytotoxic immune cell, for example a cytotoxic T-lymphocyte (CTL), capable of immunologically recognizing said first cell, and
(d) Bringing into contact the first cell and the candidate compound, and optionally the second cell, and
(e) Determining subsequent to step (d), either or both of:
   (i) expression, function and/or stability of said protein or mRNA of OR10H1, or of a variant of OR10H1, in said first cell, wherein a reduced expression, function and/or stability of said protein or mRNA of OR10H1, or of a variant of OR10H1, in said first cell contacted with the candidate compound compared to said first cell not contacted with said candidate compound indicates that the candidate compound is a compound suitable for the treatment of a disease characterized by expression of said protein or mRNA of OR10H1, or of a variant of OR10H1; and/or
   (ii) cytotoxicity of said second cell against said first cell, wherein an enhanced cytotoxicity of said second cell against said first cell contacted with the candidate compound compared to the cytotoxicity of said second cell against said first cell not contacted with the candidate compound indicates that the candidate compound is a compound suitable for the treatment of a disease characterized by expression of said OR10H 1, or the variant of OR10H1.

The reduction (or enhancement) of expression, function and/or stability or OR10H1 (or of the variant thereof), or the enhancement (or reduction) in cytotoxicity is, preferably, identified by reference to a control method, for example one practiced in the absence of any candidate compound, or with compound having a know effect on such expression, function and/or stability (such as a positive or negative control).

Said first cell is preferably a tumor cell, or a cell derived from a tumor cell.

Said candidate compound is selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

Pharmaceutical Compositions

Another aspect of the present invention pertains to a pharmaceutical composition for use in the prevention or treatment of a tumor disease. The pharmaceutical composition of the invention comprises a compound of the invention (eg., a modulator of OR10H1, or of a variant of OR10H1, as described herein), and a pharmaceutical acceptable carrier and/or excipient. Preferably, the pharmaceutical composition of the invention comprises one or more of the inhibitors or antagonists of OR10H1 (or variant thereof) as described herein. Also provided by the invention are pharmaceutical compositions, comprising the (isolated) antigen binding construct, or the (isolated) nucleic acid, or the vector, or the recombinant host cell, as described herein above; and a pharmaceutically acceptable carrier, stabilizer and/or excipient.

As used herein the language “pharmaceutically acceptable” carrier, stabilizer or excipient is intended to include any and all solvents, solubilizers, fillers, stabilizers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application, as well as comprising a compound of the invention (eg a modulator of OR10H1 or a variant thereof), can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Kolliphor® EL (formerly Cremophor EL™; BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention (e.g., a modulator of OR10H1 or a variant thereof) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, as well as comprising a compound of the invention (eg a modulator of OR10H1 or a variant thereof), generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and ex-pectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

Furthermore, the compounds of the invention (eg a modulator of OR10H1 or a variant thereof) can be administrated rectally. A rectal composition can be any rectally acceptable dosage form including, but not limited to, cream, gel, emulsion, enema, suspension, suppository, and tablet. One preferred dosage form is a suppository having a shape and size designed for introduction into the rectal orifice of the human body. A suppository usually softens, melts, or dissolves at body temperature. Suppository excipients include, but are not limited to, theobroma oil (cocoa butter), glycerinated gelatin, hydrogenated vegetable ails, mixtures of polyethylene glycols of various molecular weights, and fatty acid esters of polyethylene glycol.

For administration by inhalation, the compounds of the invention (eg a modulator of OR10H1 or a variant thereof) are typically delivered in the form of an aerosol spray from pressured container or dispenser which contains a suitable propellant, e.g., a gas such as carbon dioxide, or a nebulizer.

Systemic administration can also be by transmucosal or transdermal route and/or (other) means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions can be formulated into ointments, salves, gels, or creams as generally known in the art.

In certain embodiments, the pharmaceutical composition is formulated for sustained or controlled release of a compound of the invention (e.g., a modulator of OR10H1 or a variant thereof). Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially (including liposomes targeted to infected cells with monoclonal antibodies to viral antigens) can also be used as pharmaceutically acceptable carriers. These can be prepared according to methods known to those skilled in the art.

It is especially advantageous to formulate oral, rectal or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage of the compound of the invention (e.g., a modulator of OR10H1 or a variant thereof for use in humans. The dosage of such compounds lies preferably within a range of circulating concentrations that include the ED₅₀ with little or no toxicity. The dosage may vary within this range depending upon the dosage form employed and the route of administration utilized. For any compound used in the therapeutic approaches of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser together with instructions for administration.

In view of the above, it will be appreciated that the present invention also relates to the following items:

Item 1:A compound for use in the treatment of a disease of a subject, wherein the compound is a modulator of the expression, function and/or stability of Olfactory Receptor, Family 10, Subfamily H, Member 1 (OR10H1), or of a variant of OR10H1.

Item 2:The compound for use according to item 1, wherein the variant of OR10H1 is a protein comprising an amino acid sequence having at least 80% sequence identity to the sequence of SEQ ID NO: 25 (OR10H1 amino acid sequence).

Item 3:The compound for use according to item 1 or 2, wherein the variant of OR10H1 is selected from the group consisting of an ortholog or paralog of OR10H1, and a functional fragment of an OR10H1 protein.

Item 4:The compound for use according to any one of items 1 to 3, wherein the compound is an inhibitor or antagonist of expression, function and/or stability of OR10H1, or of the variant of OR10H1.

Item 5:The compound for use according to any of items 1 to 4, wherein the compound is selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleis acid (PNA), a genetic construct for targeted gene editing, such as a CRISPR/Cas9 construct and/or a guide nucleic acid (gRNA or gDNA) and/or tracrRNA.

Item 6:The compound for use according to any of items 1 to 5, wherein the compound is an antigen binding construct (for example, an antibody, antibody-like molecule or other antigen binding derivative, or an antigen binding fragment thereof), that binds said OR10H1, or the variant of OR10H1.

Item 7:The compound for use according to any of items 1 to 6, wherein the compound is an OR10H1 inhibitory antibody, or an inhibitory antigen binding fragment thereof, or an inhibitory antibody of a variant of OR10H1, or an inhibitory antigen binding fragment thereof.

Item 8:The compound for use according to any of items 1 to 5, wherein the compound is a nucleic acid (for example an anti-sense nucleotide molecule such as a siRNA or shRNA molecule) that binds to a nucleic acid that encodes or regulates the expression of: (i) OR10H1, or of a variant of OR10H1, or (ii) a gene that controls the expression, function and/or stability of OR10H1, or of a variant of OR10H1.

Item 9:The compound for use according to any of items 1 to 8, wherein the disease is characterized by a pathological immune response and/or expression of said OR10H1, or the variant of OR10H1.

Item 10: The compound for use according to any of items 1 to 9, wherein the disease is selected from a proliferative disease, such as cancer, preferably a cancer disease selected from lung cancer, bladder cancer, ovarian cancer, uterine cancer, endometrial cancer, breast cancer, liver cancer, pancreatic cancer, stomach cancer, cervical cancer, lymphoma, leukemia, acute myeloid leukemia, acute lymphocytic leukemia, salivary gland cancer, bone cancer, brain cancer, colon cancer, rectal cancer, colorectal cancer, kidney cancer, skin cancer, melanoma, squamous cell carcinoma, pleomorphic adenoma, hepatocellular carcinoma, and/or adenocarcinoma.

Item 11: The compound for use according to item 10, wherein the cancer is an OR10H1 positive cancer, or a cancer positive for the variant of OR10H1.

Item 12: The compound for use according to any of items 1 to 11, wherein the disease is selected from melanoma, pancreatic cancer or colorectal cancer.

Item 13: The compound for use according to any of items 1 to 12, wherein the compound is for use in enhancing an immune response in the subject, preferably for use in aiding a cell-mediated immune response in the subject such as the subject's T-cell mediated immune response, for example for treating a proliferative disease such as a cancer disease.

Item 14: The compound for use according to any of items 1 to 13, wherein the treatment comprises a transfer of cells to the subject, preferably a transfer of immune cells to the subject, more preferably an adoptive T-cell transfer.

Item 15: The compound for use according to item 14, wherein the cells are autologous cells of the subject, for example autologous immune cells, such as T-cells or Natural Killer (NK)-cells, of the subject.

Item 16: The compound for use according to any of items 1 to 15, wherein the compound is an inhibitor or antagonist of expression, function and/or stability of said OR10H1, or the variant of OR10H1, and wherein the inhibition of the expression, function and/or stability of said OR10H1, or the variant of OR10H1, enhances an immune response, preferably enhances a cell-mediated immune response in the subject such as a T-cell mediated immune response in the subject, for example for treating a proliferative disease such as a cancer disease.

Item 17: The compound for use according to item 16, wherein the immune response is enhanced by an increase in T-cell activity and/or survival.

Item 18: The compound for use according to item 17, wherein the increase in T-cell activity and/or survival is associated with the inhibition of OR10H1-mediated or OR10H1-variant-mediated cAMP signaling.

Item 19: The compound for use according to any of items 1 to 18, wherein the subject is a mouse, rat, guinea pig, rabbit, cat, dog, monkey, or preferably a human, for example a human patient.

Item 20: The compound for use according to any of items 1 to 19, wherein the treatment comprises a step of administering a therapeutically effective amount of the compound to the subject.

Item 21: An isolated antigen binding construct, capable of specifically binding to OR10H1, or of a variant of OR10H 1, optionally wherein the antigen binding construct inhibits the expression, function and/or stability of OR10H 1, or the variant of OR10H 1.

Item 22: The isolated antigen binding construct according to item 21, which comprises a sequence of an antibody variable heavy and/or light chain of an antibody obtainable from hybridoma Di-8A11-H12-E6 (DSMZ Deposition Number: DSM ACC3310, deposited 26 Oct. 2016).

Item 23: The isolated antigen binding construct according to item 22, which is an antibody obtainable from hybridoma Di-8A11-H12-E6 (DSMZ Deposition Number: DSM ACC3310, deposited 26 Oct. 2016), or an antigen binding fragment obtainable from such antibody.

Item 24: An isolated antigen binding construct comprising at least one Complementary Determining Region (CDR) 3 having an amino acid sequence with at least 80% sequence identity to an amino acid sequence selected from SEQ ID NOs. 3, 7, 11, 15, 19, and 23.

Item 25: The isolated antigen binding construct according to item 24, wherein said antigen binding construct further comprises at least one CDR1, and at least one CDR2.

Item 26: The isolated antigen binding construct according to item 24 or 25, wherein the antigen binding construct is an antibody or an antigen binding fragment thereof.

Item 27: The isolated antigen binding construct according to any one of items 24 to 26, comprising an antibody heavy chain, or an antigen binding fragment thereof, and/or an antibody light chain, or an antigen binding fragment thereof.

Item 28: The isolated antigen binding construct according to any of items 24 to 27, comprising an antibody heavy chain variable region, or an antigen binding fragment thereof, and/or an antibody light chain variable region, or an antigen binding fragment thereof.

Item 29: The isolated antigen binding construct according to any of items 24 to 28, comprising an antibody heavy chain variable region CDR1, CDR2, and CDR3, and/or an antibody light chain variable region CDR1, CDR2, and CDR3.

Item 30: The isolated antigen binding construct according to any of items 24 to 28, comprising an antibody heavy chain sequence and/or an antibody light chain sequence, or an antigen binding fragment thereof; wherein the antibody heavy chain sequence, or the fragment thereof, comprises a CDR3 having at least 80% sequence identity to an amino acid sequence selected from SEQ ID Nos. 3, 11, and 19, and/or wherein antibody light chain sequence, or the fragment thereof, comprises a CDR3 having at least 80% sequence identity to an amino acid sequence selected from SEQ ID Nos. 7, 15, and 23.

Item 31: The isolated antigen binding construct according to any one of items 27 to 30, wherein the antibody heavy chain sequence, or the fragment thereof, further comprises a CDR1 having at least 80% sequence identity to an amino acid sequence selected from SEQ ID Nos. 1, 9, and 17; and/or a CDR2 having at 80% sequence identity to an amino acid sequence selected from SEQ ID Nos. 2, 10, and 18.

Item 32: The isolated antigen binding construct according to any one of items 27 to 31, wherein the antibody light chain sequence, or the fragment thereof, further comprises a CDR1 having at least 80% sequence identity to an amino acid sequence selected from SEQ ID Nos. 5, 13, and 21; and/or a CDR2 having at least 80% sequence identity to an amino acid sequence selected from SEQ ID Nos. 6, 14, and 22.

Item 33: The isolated antigen binding construct according to any one of items 24 to 32, comprising an antibody variable chain sequence having at least 80% sequence identity to an amino acid sequence selected from SEQ ID Nos. 4, 8, 12, 16, 20, and 24.

Item 34: The isolated antigen binding construct according to any of items 24 to 33, comprising an antigen binding fragment of an antibody, wherein said antigen binding fragment comprises CDR1, CDR2 and CDR3, optionally selected from the CDR1, CDR2 and CDR3 sequences having the respective amino acid sequences of SEQ ID Nos. 1, 2, 3; or 5, 6, 7; or 9, 10, 11; or 13, 14, 15; or 17, 18, 19; or 21, 22, 23; in each case independently, optionally with not more than three or two, preferably one, amino acid substitution(s), insertion(s) or deletion(s) compared to these sequences.

Item 35: The isolated antigen binding construct according to any of items 24 to 34, wherein said CDR1 has an amino acid sequence of SEQ ID No 1, 5, 9, 13, 17 or 21, and CDR2 has an amino acid sequence of SEQ ID No 2, 6, 10, 14, 18, or 22, and CDR3 has an amino acid sequence of SEQ ID No 3, 7, 11, 15, 19, and 23; in each case independently, optionally with not more than three or two, preferably one, amino acid substitution(s), insertion(s) or deletion(s) compared to these sequences.

Item 36: The isolated antigen binding construct according to any of items 24 to 35, wherein the antigen binding construct is an antibody, or an antigen binding fragment thereof, composed of at least one, preferably two, antibody heavy chain sequences, and at least one, preferably two, antibody light chain sequences, wherein at least one, preferably both, of said antibody heavy chain sequences comprise CDR1 to CDR3 sequences having the amino acid sequences of SEQ ID NO: 1 to 3, and at least one, preferably both, of said antibody light chain sequences comprise CDR1 to CDR3 sequences having the amino acid sequences of SEQ ID NO: 5 to 7; or wherein at least one, preferably both, of said antibody heavy chain sequences comprise CDR1 to CDR3 sequences having the amino acid sequences of SEQ ID NO: 9 to 11, and at least one, preferably both, of said antibody light chain sequences comprise CDR1 to CDR3 sequences having the amino acid sequences of SEQ ID NO: 13 to 15; or wherein at least one, preferably both, of said antibody heavy chain sequences comprise CDR1 to CDR3 sequences having the amino acid sequences of SEQ ID NO: 17 to 19, and at least one, preferably both, of said antibody light chain sequences comprises CDR1 to CDR3 sequences having the amino acid sequences of SEQ ID NO: 21 to 23; in each case of a CDR independently, optionally with not more than three or two, preferably one, amino acid substitution(s), insertion(s) or deletion(s) compared to these sequences.

Item 37: The isolated antigen binding construct according to any of items 24 to 36, wherein the antigen binding construct is an antibody, or an antigen binding fragment thereof, composed of at least one, preferably two, antibody heavy chain sequence, and at least one, preferably two, antibody light chain sequence, wherein said antibody heavy chain sequence comprises a variable region having the amino acid sequence of SEQ ID NO: 4, and wherein said antibody light chain sequence comprises a variable region sequence having the amino acid sequence of SEQ ID NO: 8; or wherein said antibody heavy chain sequence comprises a variable region sequence having the amino acid sequence of SEQ ID NO: 12, and wherein said antibody light chain sequence comprises a variable region sequence having the amino acid sequence of SEQ ID NO: 16; or wherein said antibody heavy chain sequence comprises a variable region sequence having the amino acid sequence of SEQ ID NO: 20, and wherein said antibody light chain sequence comprises a variable region sequence having the amino acid sequence of SEQ ID NO: 24; in each case of a variable region sequence independently, optionally with not more than ten, nine, eight, seven, six, five, four, three, two or one, preferably not more than three, amino acid substitutions, insertions or deletions compared to these sequences.

Item 38: The isolated antigen binding construct according to any of items 24 to 37, comprising a CDR3 that has an amino acid sequence having no more than three or two, preferably one amino acid substitution(s), deletion(s) or insertion(s) relative to a sequence selected from the group consisting of SEQ ID NOs. 3, 7, 11, 15, 19, and 23.

Item 39: An isolated antigen binding construct that competes for antigen binding to an isolated antigen binding construct according to any of items 22 to 38.

Item 40: The compound for use according to any one of items 1 to 20, or the isolated antigen binding construct according to any one of items 21 to 39, which is an antibody or comprises an antibody variable heavy and/or light chain sequence, or is an antigen binding fragment thereof, wherein the construct is chimerized, optionally is humanized or murinized

Item 41: The compound for use according to any one of items 1 to 20 or 40, or the isolated antigen binding construct of any of items 21 to 39, which is a monoclonal antibody.

Item 42: The compound for use according to any one of items 1 to 20, 40 or 41, or the isolated antigen binding construct of any of items 20 to 41, wherein said compound or antigen binding construct is an IgG antibody.

Item 43: An isolated nucleic acid encoding for a compound according to any one of items 40 to 42 or an antigen binding construct according to any one of items 21 to 42, or encoding for a part or a monomer of an antigen binding construct.

Item 44: A vector comprising a nucleic acid according to item 43.

Item 45: A recombinant host cell comprising a compound according to any one of items 1 to 20 or 40 to 42, an antigen binding construct according to any one of items 21 to 42, or a nucleic acid according to item 43, or a vector according to item 44.

Item 46: The recombinant host cell according to item 45, wherein the cell is a human cell, preferably an autologous human cell.

Item 47: The recombinant host cell according to item 45, wherein the cell is a Chinese hamster ovary (CHO) cell.

Item 48: A pharmaceutical composition comprising the compound according to any one of items 1 to 20 or 40 to 42, the isolated antigen binding construct according to any of items 21 to 42, or the isolated nucleic acid according to item 43, or the vector according to item 44, or the recombinant host cell according to item 45 or 46; and a pharmaceutically acceptable carrier, stabilizer and/or excipient.

Item 49: The compound according to any one of items 1 to 20 or 40 to 42, or the isolated antigen binding construct according to any one of items 21 to 42, or a isolated nucleic acid according to item 43, or a vector according to item 43, or a recombinant host cell according to item 44 or 45, or the pharmaceutical composition according to item 47, for use in medicine, preferably for use in the diagnosis, prevention, and/or treatment of a proliferative disease, for example a disease comprising a malignant or benign tumor disease, such as a cancer.

Item 50: A method of producing a recombinant cell line capable of expressing an antigen binding construct specific for OR10H1, or for a OR10H1 variant, comprising

(a) providing a suitable host cell,

(b) providing at least one genetic construct comprising coding sequence(s) encoding the antigen binding construct according to any of items 21 to 42 or the compound according to any one of items 40 to 42,

(c) introducing into said suitable host cell said genetic construct(s),

(d) optionally, expressing said genetic construct(s) by said suitable host cell under conditions that allow for the expression of the antigen binding construct.

Item 51: A method of producing an antigen binding construct specific for OR10H1, or for a OR10H1 variant, comprising

(a) providing a hybridoma or host cell capable of expressing a compound of any one of items 40 to 42 or an antigen binding construct according to any one of items 21 to 42, for example a recombinant cell line comprising at least one genetic construct comprising coding sequence(s) encoding said compound or antigen binding construct,

(b) culturing said hybridoma or host cell under conditions that allow for the expression of the antigen binding construct.

Item 52: The method according to item 50 or 51, further comprising isolation and/or purification of said antigen binding construct.

Item 53: The method according to any one of items 50 to 52, wherein the genetic construct(s) is/are expression construct(s) comprising a promoter sequence operably linked to said coding sequence.

Item 54: The method according to any one of items 50 to 53, wherein said antigen binding construct is of mammalian origin, preferably of human origin.

Item 55: The method according to any one of items 50 to 54, wherein said suitable host cell is a mammalian cell, optionally a CHO cell.

Item 56: The method according to any of items 50 to 55, wherein said antigen binding construct is a modified antibody, wherein said modification comprises addition of a functional moiety selected from a detectable label or a cytotoxic moiety.

Item 57: A hybridoma or host cell capable of expressing a compound of any one of items 40 to 42 or an antigen binding construct according to any one of items item 21 to 42.

Item 58: A method for detecting an OR10H1 protein, or a variant of OR10H1, in a sample comprising contacting the sample with an antigen binding construct specifically binding to said OR10H1 protein, or to the variant of OR10H1, and detecting the binding between said antigen binding construct and said OR10H1 protein, or the variant of OR10H1.

Item 59: The method according to item 58, wherein the antigen binding construct is an antibody or an antigen binding fragment thereof.

Item 60: The method according to item 58 or 59, wherein the antigen binding construct is selected from a compound of any one of items 40 to 42 or an antigen binding construct according to any one of items 21 to 42.

Item 61: The method according to any one of items 58 to 60, wherein the sample is a sample of a tumor or a cancer, preferably a tumor sample of a patient suffering from a refractory/recurrent tumor disease, a metastatic tumor disease, or a multidrug resistant tumor disease.

Item 62: A method for diagnosing a resistance phenotype of a cancer disease against an immune response such as a cell-mediated immune response in a subject, the method comprising the steps of

(a) Providing a sample of tumor or cancer cells of the subject,

(b) Determining the presence or absence of protein or mRNA of OR10H1, or of a variant of OR10H1, in the tumor or cancer cells, and

(c) Diagnosing a resistance phenotype of the cancer disease against an immune response in the subject when protein or mRNA of said OR10H1, or of the variant of OR10H1, is present in the tumor or cancer cells.

Item 63: The method according to item 62, wherein the presence of an amount or level of said protein or mRNA of OR10H1, or of the variant of OR10H1, is determined in step (b) as a measured amount.

Item 64: The method according to item 63, wherein the diagnosis in step (c) is made when the presence of an amount or level of said protein or mRNA of OR10H1, or of the variant of OR10H1, in the tumor cells is greater than: (i) a measured quantity of said protein or mRNA of OR10H1, or of the variant of OR10H1, in control cells; and/or (ii) a standard or cut-off value for said quantity of said protein or mRNA of OR10H1, or of the variant of OR10H1.

Item 65: The method according to any one or items 62 to 64, wherein step (b) involves contacting the sample with a detector and/or (other) means for the detection of protein or mRNA of OR10H1, or of a variant of OR10H1, wherein said detector and/or (other) means is preferably selected from: (i) an antigen binding construct specifically binding to said protein of OR10H1, or of the variant of OR10H1, and detecting the binding between said antigen binding construct and said protein of OR10H1, or of the variant of OR10H1; and/or (ii) a nucleic acid binding to said mRNA sequence of OR10H1, or of the variant of OR10H1, and detecting the binding between said nucleic acid and said mRNA of OR10H1, or of the variant of OR10H1.

Item 66: A kit, comprising a detector and/or (other) means for the detection of protein or mRNA of OR10H1, or of a variant of OR10H1, wherein said detector and/or (other) means is preferably selected from: (i) an antigen binding construct specifically binding said protein of OR10H1, or the variant of OR10H1, and/or (ii) a nucleic acid for detecting said mRNA of OR10H1, or of the variant of OR10H1; optionally together with instructions for use and/or with one or more other components useful for said detection.

Item 67: The method according to item 65 or the kit according to item 66, wherein said detector and/or (other) means are coupled to a detectable label.

Item 68: The method according to item 65 or 67 the kit according to item 66 or 67, wherein the antigen binding construct is an antibody specifically binding to OR10H1, or to a variant of OR10H1, or an antigen binding fragment of said antibody.

Item 69: The method according to item 65, 67 or 68 or the kit according to any one of items 66 to 68, wherein the antigen binding construct is a compound of any one of items 40 to 42 or an antigen binding construct according to any one of items 21 to 42.

Item 70: The method according to any one of items 65 or 67 to 69 or the kit according to any one of items 66 to 69, wherein the nucleic acid is a PCR primer, sequencing primer, oligonucleotide probe or hybridization probe specifically binding to, such as by having a complementary sequence to, the mRNA sequence of OR10H1, or of the variant of OR10H1.

Item 71: The kit according to any one of items 66 to 70, for use in the diagnosis of a resistance of a proliferative disease, for example a cancer disease, against an immune response such as a cell-mediated immune response; preferably the kit is for use in a method according to any one of items 62 to 65 or 67 to 70.

Item 72: A method for identifying a compound suitable for the treatment of a disease characterized by expression of OR10H1, or a variant thereof, the method comprising the steps of

(a) Providing a first cell expressing a protein or mRNA of OR10H1, or of a variant of OR10H1, and

(b) Providing a candidate compound, and

(c) Optionally, providing a second cell which is a cytotoxic immune cell, for example a cytotoxic T-lymphocyte (CTL), capable of immunologically recognizing said first cell, and

(d) Bringing into contact the first cell and the candidate compound, and optionally the second cell, and

(e) Determining subsequent to step (d), either or both of:

(i) expression, function and/or stability of said protein or mRNA of OR10H1, or of a variant of OR10H1, in said first cell, wherein a reduced expression, function and/or stability of said protein or mRNA of OR10H1, or of a variant of OR10H1, in said first cell contacted with the candidate compound compared to said first cell not contacted with said candidate compound indicates that the candidate compound is a compound suitable for the treatment of a disease characterized by expression of said protein or mRNA of OR10H1, or of a variant of OR10H1; and/or

(ii) cytotoxicity of said second cell against said first cell, wherein an enhanced cytotoxicity of said second cell against said first cell contacted with the candidate compound compared to the cytotoxicity of said second cell against said first cell not contacted with the candidate compound indicates that the candidate compound is a compound suitable for the treatment of a disease characterized by expression of said OR10H1, or the variant of OR10H1.

Item 73: The method according to item 72, wherein said first cell is a tumor cell or a cell derived from a tumor cell.

Item 74: The method according to item 72 or 73, wherein said candidate compound is selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

Furthermore, in view of the above, it will be appreciated that the present invention also relates to the following additional “A” items:

Item A1: A compound for use in the treatment of a disease of a subject, wherein the compound is a modulator of the expression, function and/or stability of Olfactory Receptor, Family 10, Subfamily H, Member 1 (OR10H1), or of a variant of OR10H1.

Item A2: The compound for use according to item Al, wherein the variant of OR10H1 is selected from the group consisting of an ortholog or paralog of OR10H1, and a functional fragment of an OR10H1 protein.

Item A3: The compound for use according to item A1 or A2, wherein the compound is an inhibitor or antagonist of expression, function and/or stability of OR10H1, or of the variant of OR10H1.

Item A4: The compound for use according to any of item A1 to A3, wherein the compound is an antigen binding construct (for example, an antibody, antibody-like molecule or other antigen binding derivative, or an antigen binding fragment thereof), that binds said OR10H1, or the variant of OR10H1.

Item A5: The compound for use according to any of items A1 to A3, wherein the compound is a nucleic acid (for example an anti-sense nucleotide molecule such as a siRNA or shRNA molecule) that binds to a nucleic acid that encodes or regulates the expression of: (i) OR10H1, or of a variant of OR10H1, or (ii) a gene that controls the expression, function and/or stability of OR10H1, or of a variant of OR10H1.

Item A6: The compound for use according to any of items A1 to A5, wherein the disease is characterized by a pathological immune response and/or expression of said OR10H1, or the variant of OR10H1.

Item A7: The compound for use according to any of items A1 to A6, wherein the disease is:

(i) an OR10H1 positive cancer, or a cancer positive for the variant of OR10H1; and/or (ii) selected from melanoma, pancreatic cancer or colorectal cancer.

Item A8: The compound for use according to any of items A1 to A7, wherein the compound is an inhibitor or antagonist of expression, function and/or stability of said OR10H1, or the variant of OR10H1, and wherein the inhibition of the expression, function and/or stability of said OR10H1, or the variant of OR10H1, enhances an immune response, preferably enhances a cell-mediated immune response in the subject such as a T-cell mediated immune response in the subject, for example for treating a proliferative disease such as a cancer disease.

Item A9: An isolated antigen binding construct, capable of specifically binding to OR10H1, or of a variant of OR10H1, wherein the antigen binding construct inhibits the expression, function and/or stability of OR10H1, or the variant of OR10H1.

Item A10: An isolated antigen binding construct which is an antibody obtainable from hybridoma Di-8A11-H12-E6 (DSMZ Deposition Number: DSM ACC3310, deposited 26 Oct. 2016), or an antigen binding fragment obtainable from such antibody; wherein said antigen binding fragment specifically binds to OR10H1, or of a variant of OR10H1, and optionally inhibits the expression, function and/or stability of OR10H1, or the variant of OR10H1.

Item A11: An isolated antigen binding construct comprising at least one Complementary Determining Region (CDR) 3 having an amino acid sequence with at least 80% sequence identity to an amino acid sequence selected from SEQ ID NOs. 3, 7, 11, 15, 19, and 23; wherein said isolated antigen binding construct specifically binds to OR10H1, or of a variant of OR10H1, and optionally inhibits the expression, function and/or stability of OR10H1, or the variant of OR10H1.

Item A12: The isolated antigen binding construct according to item A11, wherein the antigen binding construct is an antibody, or an antigen binding fragment thereof, composed of at least one, preferably two, antibody heavy chain sequences, and at least one, preferably two, antibody light chain sequences, wherein at least one, preferably both, of said antibody heavy chain sequences comprise CDR1 to CDR3 sequences having the amino acid sequences of SEQ ID NO: 1 to 3, and at least one, preferably both, of said antibody light chain sequences comprise CDR1 to CDR3 sequences having the amino acid sequences of SEQ ID NO: 5 to 7; or wherein at least one, preferably both, of said antibody heavy chain sequences comprise CDR1 to CDR3 sequences having the amino acid sequences of SEQ ID NO: 9 to 11, and at least one, preferably both, of said antibody light chain sequences comprise CDR1 to CDR3 sequences having the amino acid sequences of SEQ ID NO: 13 to 15; or wherein at least one, preferably both, of said antibody heavy chain sequences comprise CDR1 to CDR3 sequences having the amino acid sequences of SEQ ID NO: 17 to 19, and at least one, preferably both, of said antibody light chain sequences comprises CDR1 to CDR3 sequences having the amino acid sequences of SEQ ID NO: 21 to 23; in each case of a CDR independently, optionally with not more than three or two, preferably one, amino acid substitution(s), insertion(s) or deletion(s) compared to these sequences.

Item A13: The isolated antigen binding construct according to item A11 or A12, wherein the antigen binding construct is an antibody, or an antigen binding fragment thereof, composed of at least one, preferably two, antibody heavy chain sequence, and at least one, preferably two, antibody light chain sequence, wherein said antibody heavy chain sequence comprises a variable region having the amino acid sequence of SEQ ID NO: 4, and wherein said antibody light chain sequence comprises a variable region sequence having the amino acid sequence of SEQ ID NO: 8; or wherein said antibody heavy chain sequence comprises a variable region sequence having the amino acid sequence of SEQ ID NO: 12, and wherein said antibody light chain sequence comprises a variable region sequence having the amino acid sequence of SEQ ID NO: 16; or wherein said antibody heavy chain sequence comprises a variable region sequence having the amino acid sequence of SEQ ID NO: 20, and wherein said antibody light chain sequence comprises a variable region sequence having the amino acid sequence of SEQ ID NO: 24; in each case of a variable region sequence independently, optionally with not more than ten, nine, eight, seven, six, five, four, three, two or one, preferably not more than three, amino acid substitutions, insertions or deletions compared to these sequences.

Item A14: An isolated (first) antigen binding construct that competes for antigen binding to an isolated antigen binding construct according to any of items A10 to A13; optionally wherein said (first) antigen binding construct inhibits the expression, function and/or stability of OR10H1, or the variant of OR10H1.

Item A15: A pharmaceutical composition comprising the compound according to any one of items A1 to A8, the isolated antigen binding construct according to any of items A9 to A14, or a nucleic acid encoding for said antigen binding or for a part or a monomer of said antigen binding construct; and a pharmaceutically acceptable carrier, stabilizer and/or excipient.

Item A16: An in vitro method for diagnosing a resistance phenotype of a cancer disease against an immune response such as a cell-mediated immune response in a subject, the method comprising the steps of
(a) Providing a sample of tumor or cancer cells of the subject,
(b) Determining the presence or absence of protein or mRNA of OR10H1, or of a variant of OR10H1, in the tumor or cancer cells, and
(c) Diagnosing a resistance phenotype of the cancer disease against an immune response in the subject when protein or mRNA of said OR10H1, or of the variant of OR10H1, is present in the tumor or cancer cells.

Item A17: The method according to item A16, wherein step (b) involves contacting the sample with a detector and/or (other) means for the detection of protein or mRNA of OR10H1, or of a variant of OR10H1, wherein said detector and/or (other) means is preferably selected from: (i) an antigen binding construct specifically binding to said protein of OR10H1, or of the variant of OR10H1, and detecting the binding between said antigen binding construct and said protein of OR10H1, or of the variant of OR10H1; and/or (ii) a nucleic acid binding to said mRNA sequence of OR10H1, or of the variant of OR10H1, and detecting the binding between said nucleic acid and said mRNA of OR10H1, or of the variant of OR10H1.

Item A18: A method for identifying a compound suitable for the treatment of a disease characterized by expression of OR10H1, or a variant thereof, the method comprising the steps of
(d) Providing a first cell expressing a protein or mRNA of OR10H1, or of a variant of OR10H1, and
(e) Providing a candidate compound, and
(f) Optionally, providing a second cell which is a cytotoxic immune cell, for example a cytotoxic T-lymphocyte (CTL), capable of immunologically recognizing said first cell, and
(g) Bringing into contact the first cell and the candidate compound, and optionally the second cell, and
(h) Determining subsequent to step (d), either or both of:
   (i) expression, function and/or stability of said protein or mRNA of OR10H1, or of a variant of OR10H1, in said first cell, wherein a reduced expression, function and/or stability of said protein or mRNA of OR10H1, or of a variant of OR10H1, in said first cell contacted with the candidate compound compared to said first cell not contacted with said candidate compound indicates that the candidate compound is a compound suitable for the treatment of a disease characterized by expression of said protein or mRNA of OR10H1, or of a variant of OR10H1; and/or
   (ii) cytotoxicity of said second cell against said first cell, wherein an enhanced cytotoxicity of said second cell against said first cell contacted with the candidate compound compared to the cytotoxicity of said second cell against said first cell not contacted with the candidate compound indicates that the candidate compound is a compound suitable for the treatment of a disease characterized by expression of said OR10H1, or the variant of OR10H1.

Item A19: The compound for use according to any of items A1 to A8, the isolated antigen binding construct according to any of items A9 to A14, the pharmaceutical composition according to item A15, the in vitro method according to item A16 or A17, or the method according to item A18, wherein OR10H1is a protein comprising an amino acid sequencing according to SEQ ID NO 25, and the variant of OR10H1 is a protein comprising an amino acid sequence having at least 95% sequence identity to the sequence of SEQ ID NO 25.

The present invention will now be further described in the following examples with reference to the accompanying figures and sequences, nevertheless, without being limited thereto. For the purposes of the present invention, all references as cited herein are incorporated by reference in their entireties.

### IN THE FIGURES

FIG. 1AA: OR10H1 is expressed by various cancer cell-lines. RT-PCR shows expression of OR10H1 by the cell lines: M579-A2 (melanoma), PANC-1 (PDAC) and SW480 (colorectal), but not by the myeloma cell line KMM-1.

FIG. 1 cont.: OR10H1 prevents lysis of solid tumors by TILs. AB M579-A2 cells were transfected with the siRNA sequences for OR10H1 and mRNA was measured by RT-PCR after 72 h. Beta-actin served as a control. B, C Killing assays of M579-A2 with two different TIL-cultures. B M579-A2-luc cells, which were transfected with individual (s1-s4), pooled OR10H1 siRNA or control siRNA (PD-L1 as positive and non-specific as negative control) and TIL-mediated lysis was measured by Luc-CTL cytotoxicity assay (cumulative data). C Chromium-release assay showing specific lysis of M579-A2 by TIL209 at different E:T ratios after transfection with OR10H1 siRNA 1 (Δ), positive control PD-L1 siRNA (∘) or control siRNA (▪). D Luc-CTL assay with an autologous pair of melanoma (M615-luc) and TIL (TIL615) conducted in parallel to C (cumulative data,). E, F Chromium-release assay showing, respectively lysis of SW480 colorectal cancer and PANC-1 pancreatic adenocarcinoma cells by patient-derived HLA-matched TILs at different E:T ratios upon OR10H1 (Δ), PD-L1 positive control (∘) or control (▪) knockdown. All experiments were performed in triplicates and are representative (if not indicated otherwise) of at least three independent experiments. Error bars denote±SEM, and statistical significance was calculated using unpaired, two-tailed Student's t-test.

FIG. 2: OR10H1 inhibits TIL type I cytokine secretion and induces apoptosis. A Cumulative data of type I vs. type II cytokine secretion of TIL412 after 20 h co-culture with OR10H1 knock-down (siRNA1 or “s1”) or OR10H1 expressing (control siRNA) M579-A2 measured by Luminex assay. B ELISpot assay showing the number of IFN-γ secreting TILs (spot numbers) after co-culture with OR10H1 knockdown M579-A2 (or control siRNA). TILs without stimulation were used as a negative control. C cumulative data of apoptosis induction (measured by FACS staining for Annexin V⁺) in CD8⁻ TILs after co-culture (6 h) with OR10H1 knock-down (siRNA1 or siRNA pool) or OR10H1 expressing (control siRNA) M579-A2. TILs stimulated with PMA/ionomycin and unstimulated TILs (no melanoma encounter) were used as positive and negative controls, respectively. A and C show cumulative data from three independent experiments. B shows representative data of two independent experiments (performed in triplicates). Error bars denote±SEM, and statistical significance was calculated using unpaired, two-tailed Student's t-test.

FIG. 3: OR10H1 functions as an immune checkpoint in vivo. A Stable OR10H1 knock-down M579-A2 (transduced with OR10H1-targeting shRNA) or control transduced M579-A2 (non-targeting sequence shRNA; NTS) were injected subcutaneously (mixed with matrigel) into the left and right flank of NSG mice. On day 2 and 9, mice (n=6) received adoptive cell transfer of TIL412 (i.v. injection) and the tumor growth was measured. Mice (n=4) with tumors but without ACT serve as a control group for tumor growth. B, C Tumor growth curves showing mean±SEM tumor volume of OR10H1-negative (ie OR10H1 knockdown; kd) or OR10H1 -positive (ie NTS shRNA control) M579-A2 tumors in TIL412-treated mice, ie, with ACT (B), or in the growth control group without ACT (C). Statistical difference was calculated using the Mann-Whitney U test.

FIG. 4: OR10H1 reduces Lck and increases CREB activity by cAMP signaling via PKA in TILs. A, B TIL412 were co-cultured with OR10H1-positive (control siRNA) or OR10H1-negative (OR10H1 siRNA) M579-A2 cells for 10 h and mRNAs were extracted from the respective TILs for transcriptome analysis by RNA sequencing. A Smear plot showing differential gene expression (log 2 of fold change) between OR10H-negative (kd)− and OR10H1-postive, control siRNA)-tumor cell-treated TILs versus the average expression (log of count per million). LogFC cutoff at ±0.5 is represented by horizontal lines. Genes with a false discovery rate (FDR) above 0.05 (depicted in gray) were excluded from subsequent analysis. B Functional enrichment analysis (Ingenuity pathway analysis; IPA) based on top upregulated (LogFC>0.5) and downregulated (LogFC<−0.5) genes with FDR ≤0.05. Differential gene expression-associated functions were selected based on enrichment p-value (−log of p-value; threshold=−1.3) and activation Z-score (threshold ±2). RNA-Sequencing was performed in duplicates. C, D, E, F Phospho pathway analysis for key signaling proteins in TILs after co-culture (up to 2 h) with OR10H1-positive (ie control siRNA) and OR10H1-negative (ie OR10H1 siRNA) M579-A2. Phosphoplex analysis showing phosphorylation state changes in TILs upon encountering OR10H1-positive- or OR10H1-negative M579-A2 tumor cells (log 2 ratio) of classical (C) or T cell receptor (TCR)-associated (D, E) signaling proteins (CREB and Lck) compared to unstimulated TILs. In each case, TILs stimulated with PMA and ionomycin serve as positive control. F Immunoblot analysis showing phospho-CREB (Ser133), phospho-ATF1, phospho-PKA (Thr197) and phospho-Lck (Tyr505) levels in OR10H1-positive tumor cell-treated, OR10H1-negative tumor cell-treated, PMA and iono-mycin-treated or unstimulated TILs using the according phospho-specific antibodies. Beta-actin was used as a loading control. C, D and E are cumulative data of three independent experiments. F is representative of three independent experiments. Mean±SEM are shown, unless stated otherwise, and statistical significance was calculated using unpaired, two-tailed Student's t-test. G Lck inhibition by a small molecule abrogates OR10H1 knockdown effect on T-cell mediated cytotoxicity (as reflected the ratio of cytotoxicity/viability) of melanoma cells. H Western blot showing that olfactory receptor signalling activates a unique G protein (G-alpha-Olf) and subsequently adenylate cyclase type III. I In the presence of TILs the cAMP response in M579-A2 cells is reduced if OR10H1 is knocked-down on the melanoma cells.

FIG. 5: Generation of monoclonal blocking antibodies against OR10H1. Mother clones of OR10H1-blocking antibodies were generated by genetic immunization of three rats with an OR10H1-IgG construct and screening for binding and blocking activity. The four most promising mother clones were enriched and tested for their impact on TIL-mediated killing by Luc-CTL cytotoxicity assay (A and B) and for two of such clones in an IncuCyte cytotoxicity assay (C and D), based on caspase activation. A, B Blocking efficacy was tested using different antibody concentration (0-500 μg/ml) for the co-culture of TIL412 with OR10H1⁻- or OR10H1⁻M579-A2-luc, compared to pre-immunization rat serum. IncuCyte cytotoxicity assays depicting the impact of mother clones 8A1 l (C) and 1B11 (D) on TIL-mediated induction of apoptosis in M579-A2 over 48 hours (measurement every 5 min). The Y axis represents the number of green object counts (event of caspase-3 activation) per well. E, F, G Summary of screening for monoclonal antibodies derived from aforementioned mother clones. Luc-CTL cytotoxicity assays showing the impact of monoclonal supernatants (unknown concentration) on the TIL-mediated killing of wild-type (E), control knockdown (F) or OR10H1 knockdown (G) M579-A2-luc. A and B are representative of two independent experiments. C and D are exemplary experiments. E, F and G are results from one screening. A-E were performed in triplicates, F and G were performed in duplicates. Error bars denote±SEM, and statistical significance was calculated using unpaired, two-tailed Student's t-test with *p≤0.05; **p≤0.01; ***p≤0.001; ****p≤0.0001. Clone IDs: A=1C3, B=1B11, C=8A11, D=4B4, A-1=1C3-B2, A-2=1C3-A9, D-1=4B4-A10, C-1=8A11-G12, C-2=8A11-E7, B-1=1B11-B12 and B-2=1B11-B9.

FIG. 6: Detection of OR10H1 using an antibody of the invention. A Surface expression of OR10H1 on melanoma M579-A2 transfected with non-specific control siRNA or OR10H1 siRNA 1 detected with post-immunisation polysera against OR10H1. B Surface expression of OR10H1 detected by the antibody purified from clone Di-8A11-H12-E6 on M579-A2 melanoma cells that express OR10H1 (determined by RT-PCR), but not detected on KMM-1 myeloma cells that do not express OR10H1 (determined by RT-PCR).

FIG. 7: Amino acid sequences of certain antibodies of or for use in the present invention. A. Sequence of the amino acid sequence of the heavy and light chains of antibody 1C3-A1-A1; B. Sequence of the amino acid sequence of the heavy and light chains of antibody 1C3-Al-A2; C. Sequence of the amino acid sequence of the heavy and light chains of anti-body 8A11-B9-A1.

And in the Sequences:

TABLE 1D
Antibody Sequences

| SEQ ID NO: | Antibody | Chain | Region | Amino Acid Sequence |
|---|---|---|---|---|
| 1 | 1C3-A1-A1 | heavy | CDR1 | FTFDNAW |
| 2 | 1C3-A1-A1 | heavy | CDR2 | IKAKSNNYAT |
| 3 | 1C3-A1-A1 | heavy | CDR3 | TRLYYD |
| 4 | 1C3-A1-A1 | heavy | variable domain | EVQLVETGGNLVQPGKSL |
| | | | | KLTCATSGFTFDNAWMH |
| | | | | WVRQSPEKQLEWVAQIKA |
| | | | | KSNNYATYYAESVKGRFTI |
| | | | | SRDDSKSSVYLQMNRLKE |
| | | | | EDTAIYYCTRLYYDWGQG |
| | | | | VMVTVSS |
| 5 | 1C3-A1-A1 | light | CDR1 | QDIGNY |
| 6 | 1C3-A1-A1 | light | CDR2 | RAT |
| 7 | 1C3-A1-A1 | light | CDR3 | LQHKQYPL |
| 8 | 1C3-A1-A1 | light | variable domain | DIQMTQSPSSISVSLGDRFT |
| | | | | FTCRASQDIGNYLSWFQQ |
| | | | | KPEKSPKLMIYRATNLEDG |
| | | | | VPSRFSGSRSGSDYSLTINS |
| | | | | LESEDTGIYFCLQHKQYPL |
| | | | | TFGSGTKLEIKR |
| 9 | 1C3-A1-A2 | heavy | CDR1 | FTFSNYD |
| 10 | 1C3-A1-A2 | heavy | CDR2 | ISHSGDSTYF |
| 11 | 1C3-A1-A2 | heavy | CDR3 | HGVYTN |
| 12 | 1C3-A1-A2 | heavy | variable domain | EVQLQESGGGLIQPGRSLK |
| | | | | LSCAASGFTFSNYDMAWV |
| | | | | RQAPTKGLEWVASISHSGD |
| | | | | STYFRASVKGRFTVSRDNA |
| | | | | KSSLYLQMDSLRSEDTATY |
| | | | | YCARHGVYTNYGIWFAY |
| | | | | WGQGTLVTVSS |
| 13 | 1C3-A1-A2 | light | CDR1 | QDIGNY |
| 14 | 1C3-A1-A2 | light | CDR2 | RAT |
| 15 | 1C3-A1-A2 | light | CDR3 | LQHKQYPL |
| 16 | 1C3-A1-A2 | light | variable domain | DIQMTQSPSSISVSLGDRFT |
| | | | | FTCRASQDIGNYLSWFQQ |
| | | | | KPEKSPKLMIYRATNLEDG |
| | | | | VPSRFSGSRSGSDYSLTINS |
| | | | | LESEDTGIYFCLQHKQYPL |
| | | | | TFGSGTKLEIKR |
| 17 | 8A11-B9-A1 | heavy | CDR1 | FTFSSAW |
| 18 | 8A11-B9-A1 | heavy | CDR2 | IKGKSNNYAT |
| 19 | 8A11-B9-A1 | heavy | CDR3 | TWFGPMDA |
| 20 | 8A11-B9A1 | heavy | variable domain | EVQLQESGGRLVQPGKSL |
| | | | | KLTCAASGFTFSSAWIHW |
| | | | | VRQSPEKQLEWVAQIKGK |
| | | | | SNNYATYYAESVKGRFTIS |
| | | | | RDDSKSSVYLQMNSLKEE |
| | | | | DTAIYYCTWFGPMDAWG |
| | | | | QGASVTVSS |
| 21 | 8A11-B9-A1 | light | CDR1 | KSLLHNNGKTF |
| 22 | 8A11-B9-A1 | light | CDR2 | WMS |
| 23 | 8A11-B9-A1 | light | CDR3 | QQFLEYPL |
| 24 | 8A11-B9-A1 | light | variable domain | DIVMTQGALPNPVPSGESA |
| | | | | SITCQSSKSLLHNNGKTFL |
| | | | | NWYLQRPGQSPQLLIYWM |
| | | | | STRASGVSDRFSGSGSGAD |
| | | | | FTLKISSVEAEDVGVYYCQ |
| | | | | QFLEYPLTFGSGTKLEIKR |

SEQ ID NO 25: shows the OR10H1 Full Length Protein Sequence:
MQRANHSTVTQFILVGFSVFPHLQLMLFLLFLLMYLFTLLGNLLIMATVWSERSLHTP
MYLFLCALSVSEILYTVAIIPRMLADLLSTQRSIAFLACASQMFFSFSFGFTHSFLLTVM
GYDRYVAICHPLRYNVLMSPRGCACLVGCSWAGGLVMGMVVTSAIFHLAFCGHKEI
HHFACHVPPLLKLACGDDVLVVAKGVGLVCITALLGCFLLILLSYAFIVAAILKIPSAE
GRNKAFSTCASHLTVVVVHYGFASVIYLKPKSPQSLEGDTLMGITYTVLTPFLSPIIFSL
RNKELKVAMKKTFFSKLYPEKNVMM
SEQ ID NO: 26 to 34 show OR10H1 siRNA/shRNA sequences (see table E1/E2 below).

### EXAMPLES

### Example 1

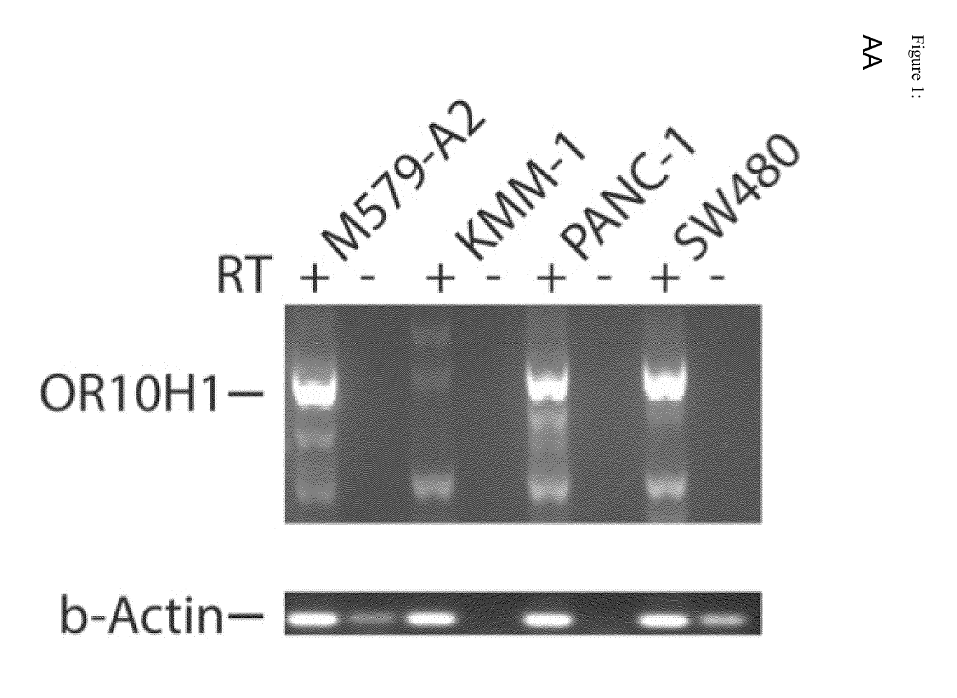

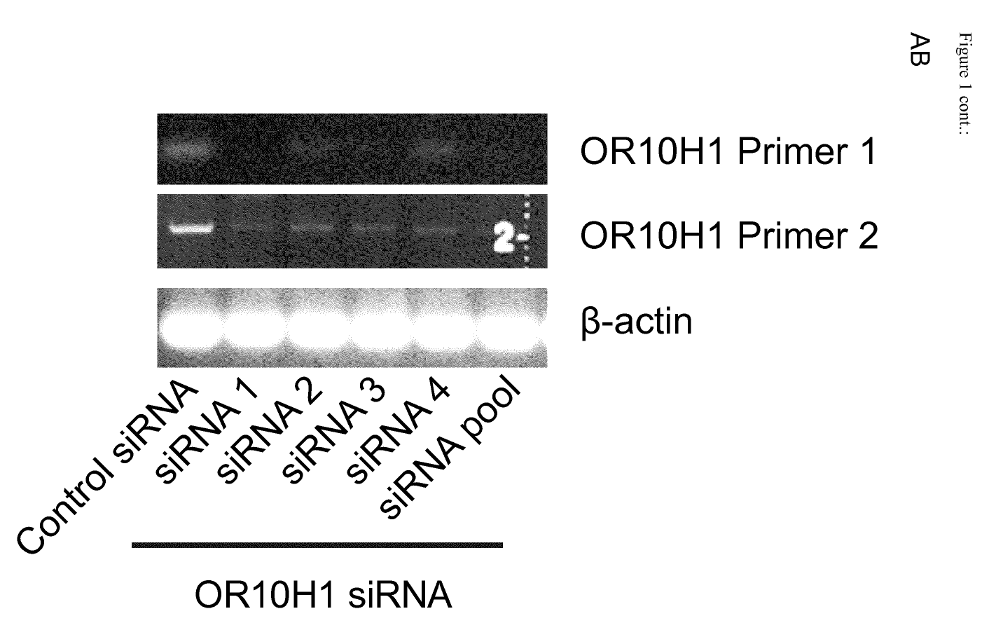

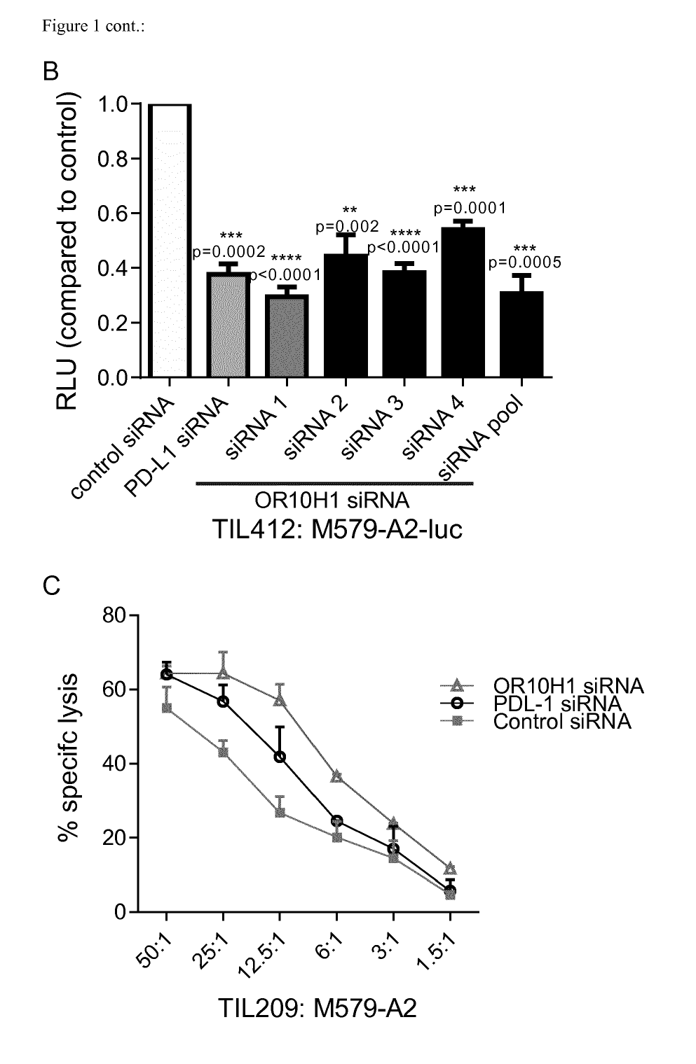

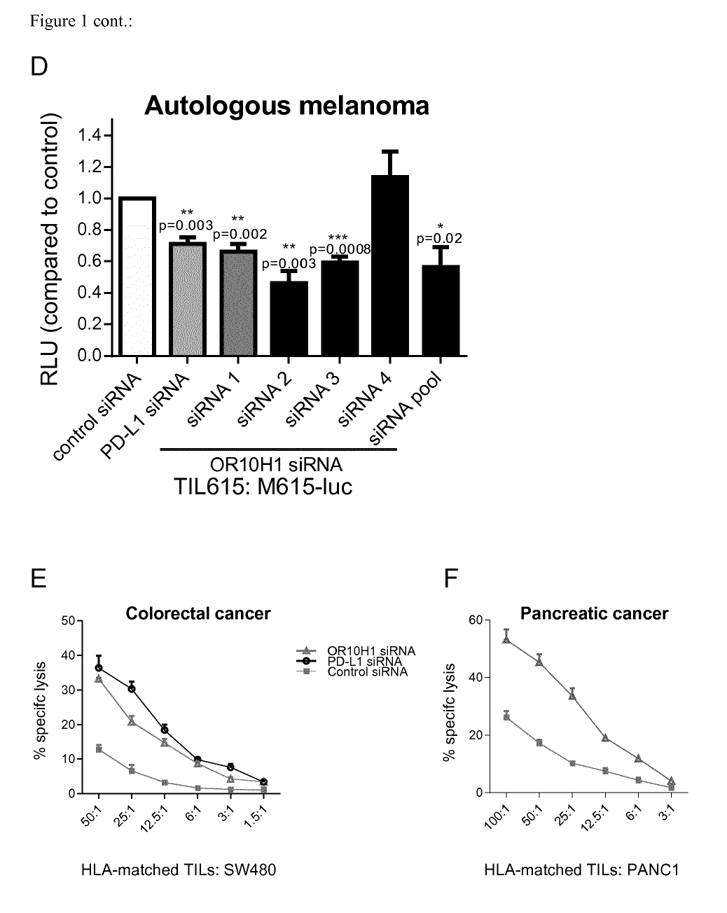

### OR10H1 Knock-Down Increases TIL-Mediated Killing of Solid Tumors (FIG. 1)

RT-PCR data and expression database searches suggest that the cell surface-expressed gene OR10H1 is expressed by melanoma, PDAC and colorectal cancer. Therefore, two OR10H1-specific PCR primers are tested by sequencing the respective RT-PCR amplicons according to standard procedures. The results show that OR10H1 is expressed by the following cells: M579-A2 (melanoma) (Machlenkin et al, 2008; Cancer Res 68:2006-13), PANC-1 (PDAC) and SW480 (colorectal) (both, ATCC), but not expressed by KMM-1 (myeloma) (Namba et al, 1989; In Vitro Cell Dev Biol 8:723-9 (FIG. 1AA).

SMART pool siRNA against OR10H1 (GE Dharmacon) were tested (by RT-PCR) for their activity in downregulating OR10H1 mRNA in M579 (melanoma) cells. Following transfection into M579-A2 cells, all siRNAs show a knock-down of OR10H1 (FIG. 1AB). siRNA 1 shows a complete absence of OR10H1 mRNA, siRNA 3 and pooled OR10H1 siRNAs show a strong reduction, siRNA 2 and 4 show a weaker but clear reduction in OR10H1 transcription. Details of the respective siRNAs are set out in Table E1.

TABLE E1
Exemplary siRNAs against ORH10H1

| | | GE Dharmacon | SEQ ID |
|---|---|---|---|
| Name | Sequence | order # | No. |
| OR10H1 | GGAGACACCUUGAUGGGCA | D-020479-01 | 26 |
| siRNA 1 | | | |
| OR10H1 | AGUAAACUCUACCCAGAAA | D-020479-02 | 27 |
| siRNA 2 | | | |
| OR10H1 | GCAGAGAGCCAAUCACUCC | D-020479-03 | 28 |
| siRNA 3 | | | |
| OR10H1 | GGUCGUGCACUAUGGCUUU | D-020479-04 | 29 |
| siRNA 4 | | | |
| OR10H1 | | M-020479-01 | |
| pool | | | |

The inventors were surprised to find that all siRNAs show an impact on TIL412-mediated killing of M579-A2-luc at 5:1 Effector to Target cell (E:T) ratio (FIG. 1 B). Transfection of M579-A2-luc cells with OR10H1 siRNA 1 increased HLA-matched patient-derived tumor-infiltrating lymphocyte (TIL, clone 412)-mediated killing to 70% (30% remaining melanoma cells compared to a scrambled negative control siRNA; p=<0.0001). This increase in TIL-mediated lysis is stronger than for PD-L1 positive control knock-down (39% remaining cells). Overall, the effect on the phenotype is comparable to the knock-down efficacy on OR10H1 mRNA. Only siRNA 3 shows an effect on M579-A2-luc viability (without the presence of TILs; data not shown).

TIL-mediated lysis of M579-A2 was validated in chromium release assay (4 h co-culture). Another patient-derived and HLA-matched TIL culture (TIL209) was used to show that the knock-down effect on TIL-mediated killing is not depending specifically on TIL412. Knock-down of OR10H1 (siRNA 1) strongly increased the lysis of M579-A2-luc in all effector to target (E:T) ratios compared to the negative control siRNA (57% to 27% specific lysis at 12.5 E:T ratio; FIG. 1C). Indeed, this effect of OR10H1 knock-down was stronger than the PD-L1 positive control knock-down (41% specific lysis at 12.5 E:T ratio; FIG. 1C).

OR10H1 knock-down increased TIL-mediated killing in an autologous melanoma setting. For this, M615 tumor cells and TIL615 were derived from the same patient, and M615 was stably transfected with luciferase to produce M615-luc. The basic killing of M615-luc by TIL615 was very low. However, knock-down of OR10H1 increased TIL-mediated killing compared to the negative control siRNA (FIG. 1D).

Furthermore, it was tested whether OR10H1 knock-down also has an effect in other cancers. Surprisingly, OR10H1 knock-down increased the TIL-mediated tumor cell killing in colorectal cancer (CRC) and pancreatic cancer cells. SW480 (ATCC) and PANC-1 cells were analyzed for their expression of OR10H1 (see above) and the knock-down efficacy of the siRNAs were validated. The CRC cell line SW480 and the PANC-1 cell line were each co-cultured with corresponding HLA-matched patient-derived TILs for chromium release (4 h co-culture). OR10H1 siRNA 1 almost tripled the lysis of SW480 by TILs in all effector to target ratios compared to the negative control siRNA (33% to 12.8% specific lysis at 50:1 E:T ratio; FIG. 1E). Indeed, knock-down of OR10H1 was almost as effective as knock-down of PD-L1 positive control on TIL-mediated lysis (FIG. 1E). Such effect was also surprising to see in pancreatic cancer cells: knock-down of OR10H1 (siRNA 1) doubled the lysis of PANC-1 by TILs in all effector to target ratios compared to the negative control siRNA (53% to 26% specific lysis at 50:1 E:T ratio; FIG. 1F)

The effects of siRNA knock-down on TIL-mediated killing and lysis in a chromium release assay were investigated as described in Khandelwal et al, 2015 (EMBO Mol Med 7:450-63). Tumor-infiltrating lymphocytes 412 and 209 microcultures were expanded from an inguinal lymph node of a melanoma patient as described in Dudley et al, 2010 (Clin Cancer Res 16:6122-31). M615, TIL615 and other patient-derived TIL cells were obtained analogously as described in Dudley et al, 2010. M579-A2-luc cells and M615-luc cells were produced from M579-A2 and M615, respectively (Machlenkin et al, 2008; Cancer Res 68:2006-13).

### Example 2

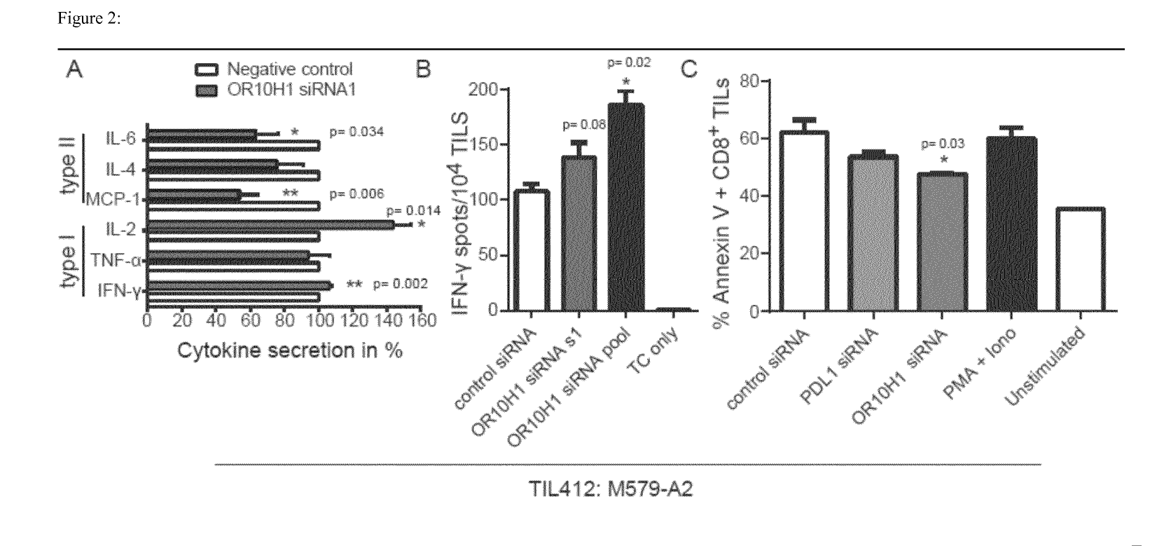

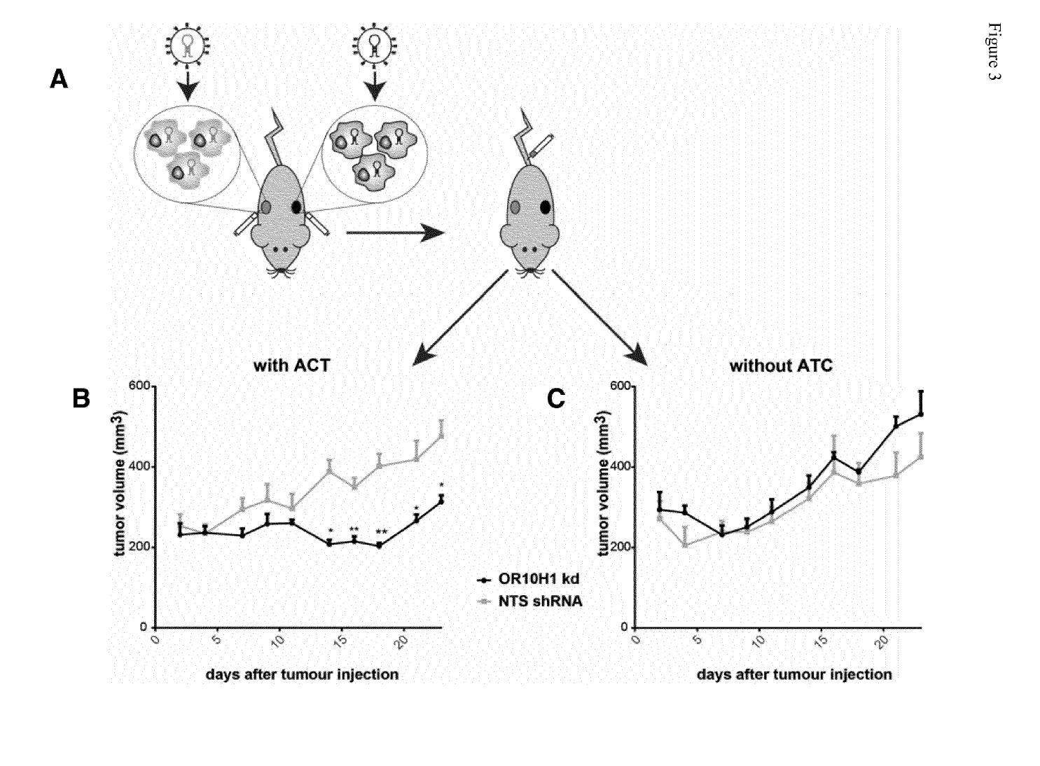

### OR10H1 Knock-Down Increases TIL Activity and Survival (FIG. 2)

A co-culture of M579-A2 and TIL412 leads to a switch in cytokines secreted by TILs (from immune-suppressive type II to immune-activating type I), showing an increase in anti-tumor response. Cells were co-cultured for 20 hours and the cytokine concentrations in the supernatant were measured by Luminex as described in Khandelwal et al, 2015. As controls unstimulated TILs (no tumor cells) and over-stimulated TILs (PMA and ionomycin) were used (not shown).

As expected unstimulated TILs did not secrete considerable amounts of cytokines, whereas over-stimulated TILs secreted dramatically increased amounts. However, the inventors were surprised to observe that knock-down of OR10H1 on melanoma increased type I cytokine secretion by TIL412 (FIG. 2A). IFN-γ secretion increased significantly from 2825 to 3037 pg/ml (increase of 8%; p=0.0236) compared to the negative control. IL-2 secretion significantly increased from 1079 to 1701 pg/ml (increase of 58%; p=0.045). Furthermore, knock-down of OR10H1 was found to surprisingly decrease type II cytokine secretion by TIL412 (FIG. 2A). MCP-1 secretion significantly decreased from 6658 to 4035 pg/ml (decrease of 39%; p=0.016). IL-6 secretion significantly decreased from 49.4 to 31 pg/ml (decrease of 37%; p=0.028). IL-4 secretion decreased from 14.75 to 11.8 pg/ml (20%; trend).

Knock-down of OR10H1 increased the number of IFN-γ producing TILs. Melanoma cells and TILs were co-cultured for 20 h and the number of IFN-γ producing cells was measured using ELISpot as described in Khandelwal et al, 2015. Although TILs did not produce IFN-g without interaction with melanoma cells, knock-down of OR10H1 (pooled siRNA) significantly increased the number of IFN-g spots/104 TILs from 107.75 to 187.75 (increase of 73%; p=0.02; FIG. 2B).

Without being bound by theory, the effect of OR10H1 knock-down (in melanoma cells) on TIL activity appeared to depend on the HLA-TCR interaction (not shown). The inventors then compared the amounts of IFN-γ after the co-culture (6 h) of M579-A2 melanoma cells with TILs. If melanoma cells did not express HLA-A2 (M579), IFN-γ secretion was abrogated regardless of the knock-down of OR10H1. However, knock-down of OR10H1 on melanoma cells surprisingly reduced the induction of apoptosis in TILs after co-culture suggesting that OR10H1 knock-down prolonged survival and/or increased proliferation of TILs. Melanoma cells and TILs were co-cultured for 6 h and the percentage of Annexin V-positive CD8+ TILs (FACS staining) were calculated, in each case according to standard procedures. TILs which were not co-cultured or activated showed 35% of Annexin V-positive CD8+ T cells. Over-activation with PMA and ionomycin resulted in 60% apoptotic CD8+ TCs. Interestingly, co-culture with OR10H1-positive M579-A2 (scrambled siRNA control) increased the percentage of apoptotic CD8+ TCs to 62%. Surprisingly, knock-down of OR10H1 prior to co-culture reduced the number of apoptotic CD8+ TILs to 47% (FIG. 2C). The induction of apoptosis (compared to unstimulated TILs) is half as large if OR10H1 is not expressed in the melanoma cells (p=0.032). Knock-down of PD-L1 decreased the percentage of apoptotic CD8+ TCs to 53% (non-significant compared to scrambled siRNA control)

### Example 3

### OR10H1 Functions as an Immune Checkpoint In Vivo (FIG. 3)

In order to assess OR10H1 function in vivo, a stable OR10H1 knock-down M579-A2 line was generated using lentiviral shRNA particles (MISSION shRNA for OR10H1; Sigma Aldrich), analogously as described in Khandelwal et al, 2015 for the stable CCR9 knockdown in M579-A2. The stable knock-down of OR10H1 increased the specific lysis of M579-A2 by TIL412 and TIL209 in vitro (chromium release) compared to negative target sequence (NTS) control-transduced M579-A2 (not shown). ShRNA 4 showed the strongest impact on OR10H1 expression and functionality, whereas stable knock-down of OR10H1 did not affect viability or proliferation of M579-A2 in vitro (not shown). Details of the respective shRNAs are set out in Table E2.

TABLE E2
Exemplary shRNAs against ORH10H1

| | | | SEQ |
|---|---|---|---|
| | | Sigma Aldrich | ID |
| Name | Sequence | order# | No. |
| OR10H1 | GTTCCTGCTGATGTACCTGTT | TRCN0000011786 | 30 |
| shRNA 1 | | | |
| OR10H1 | TGCGCTACAACGTGCTCATGA | TRCN0000357706 | 31 |
| shRNA 2 | | | |
| OR10H1 | TGGCTTTGCCTCCGTCATTTA | TRCN0000357707 | 32 |
| shRNA 3 | | | |
| OR10H1 | TCTGCTGAAGGTCGGAACAAG | TRCN0000357708 | 33 |
| shRNA 4 | | | |
| OR10H1 5 | ACACAAGGAGATCCACCATTT | TRCN0000357775 | 34 |
| shRNA | | | |

OR10H1 knock-down and NTS control-transduced M579-A2 cells (in matrigel) were injected subcutaneously into the flanks of immunodeficient (NOD scid gamma; NSG) mice (FIG. 3A) and on day 2 and 9 after tumor inoculation, TIL412 cells were injected intravenously (adoptive cell transfer; ACT). Subsequently the tumor size was measured for 24 days. A control group of mice was injected with tumor cells but did not receive ACT, so as to validate the effect of OR10H1 knock-down on tumor growth without the presence of TILs.

Tumor growth is strongly reduced in OR10H1 knock-down (kd) tumors (ie, OR10H1-negative tumors) after ACT with TIL412 but not without the ACT (FIG. 3B and C). On day 7 (273 mm³) the tumors start to grow regardless of the presence or absence of OR10H1. There is no significant difference in tumor growth of OR10H1 knock-down M579-A2 compared to that of the NTS shRNA control group (FIG. 3C). In contrast, and to the surprise of the inventors, if the mice were i.v. injected with TIL412 as ACT, M579-A2 tumor growth of OR10H1-negative (kd), but not growth of shRNA control M579-A2 tumors (ie, OR10H1-negative), was significantly reduced after day 11 (FIG. 3B). OR10H1 knock down tumors significantly reduced in volume from 260 to 207 mm³ between day 11 and 14 after inoculation. In contrast, and over the same period, the NTS control tumors grew substantially from 296 to 389 mm³ (p=0.013). On day 18 the average OR10H1-negative tumor (kd) had a volume of only 203 mm³ compared to 401 mm³ for the average size of NTS control (ie OR10H1-positive) tumors (p=0.004). Although after day 18, the OR10H1-negative tumors do start to grow again, their tumor mass remains significantly reduced compared to NTS control (ie OR10H1-positive) tumors.

### Example 4

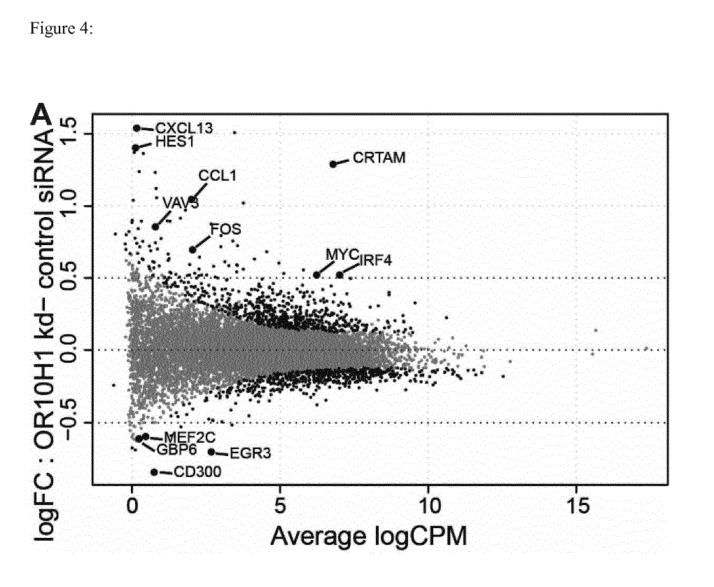

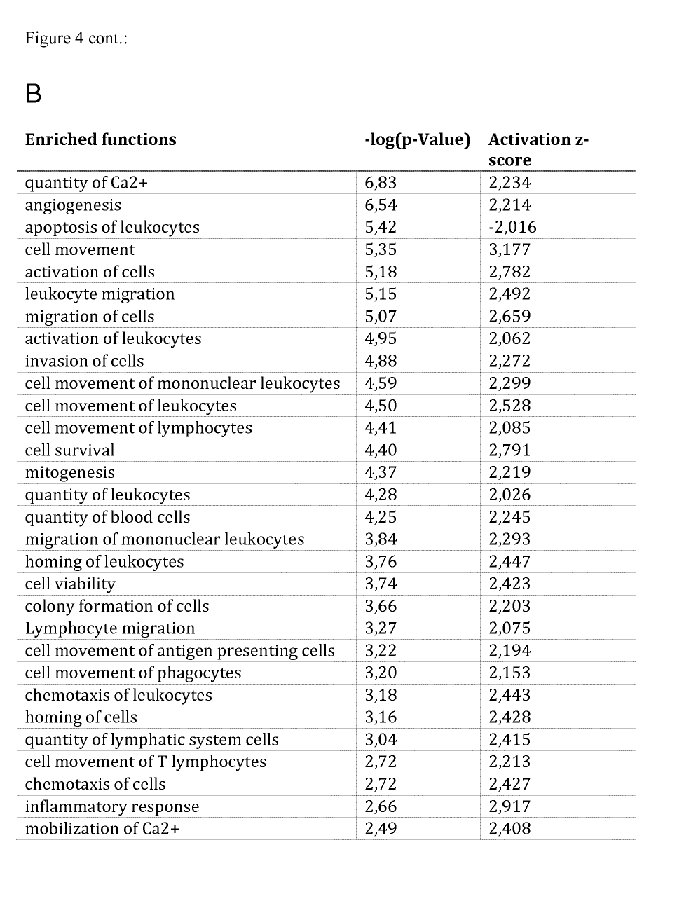

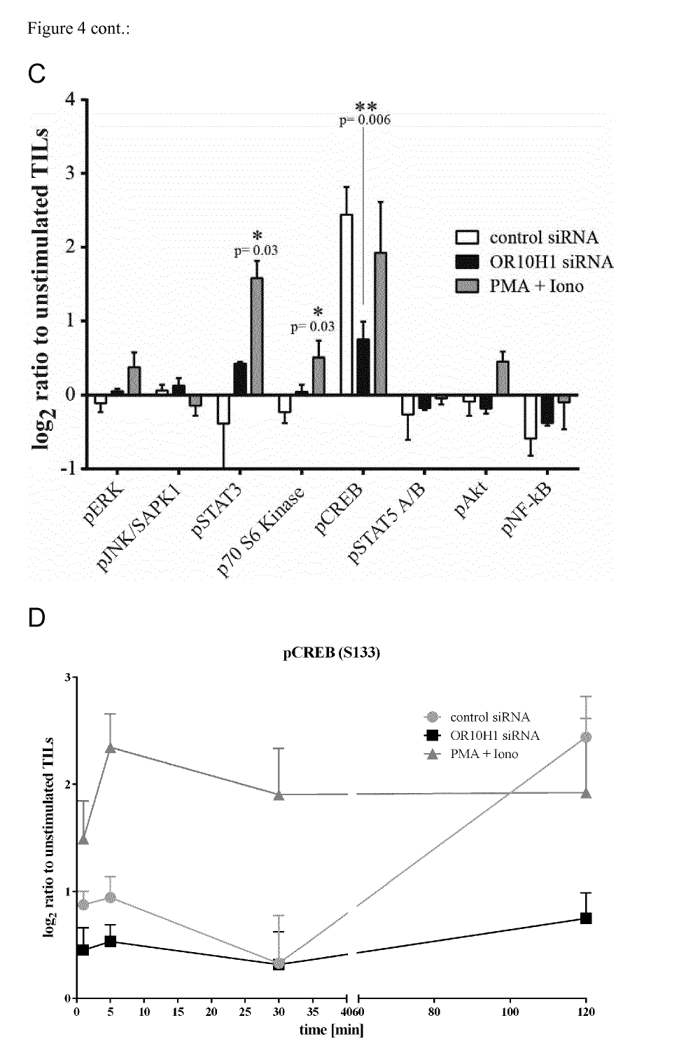

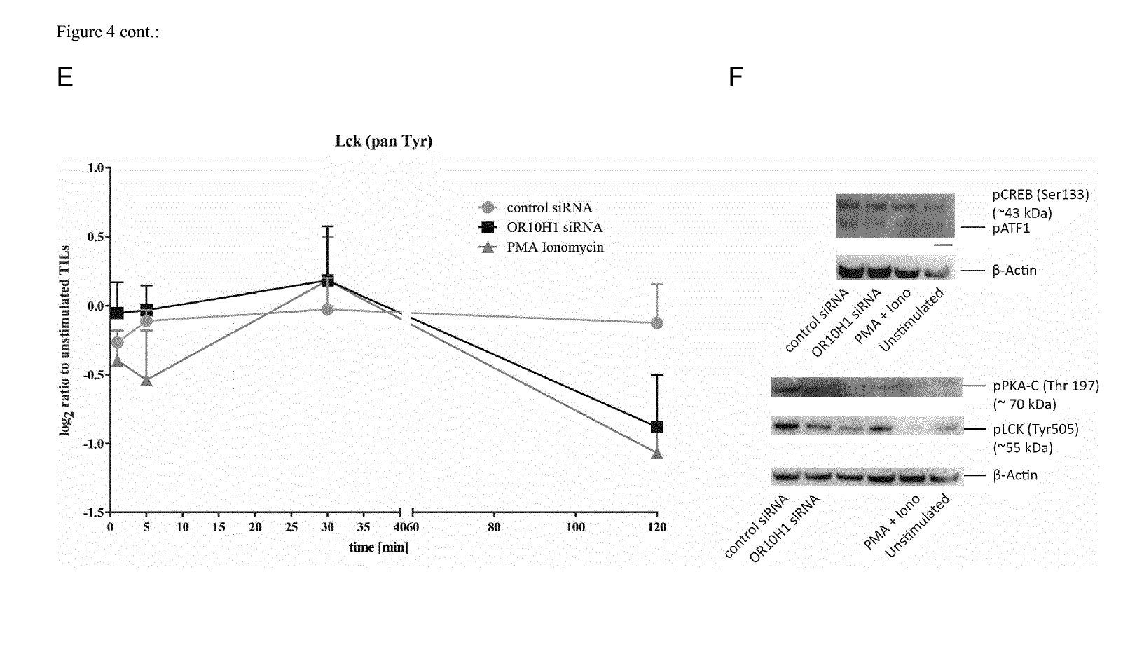

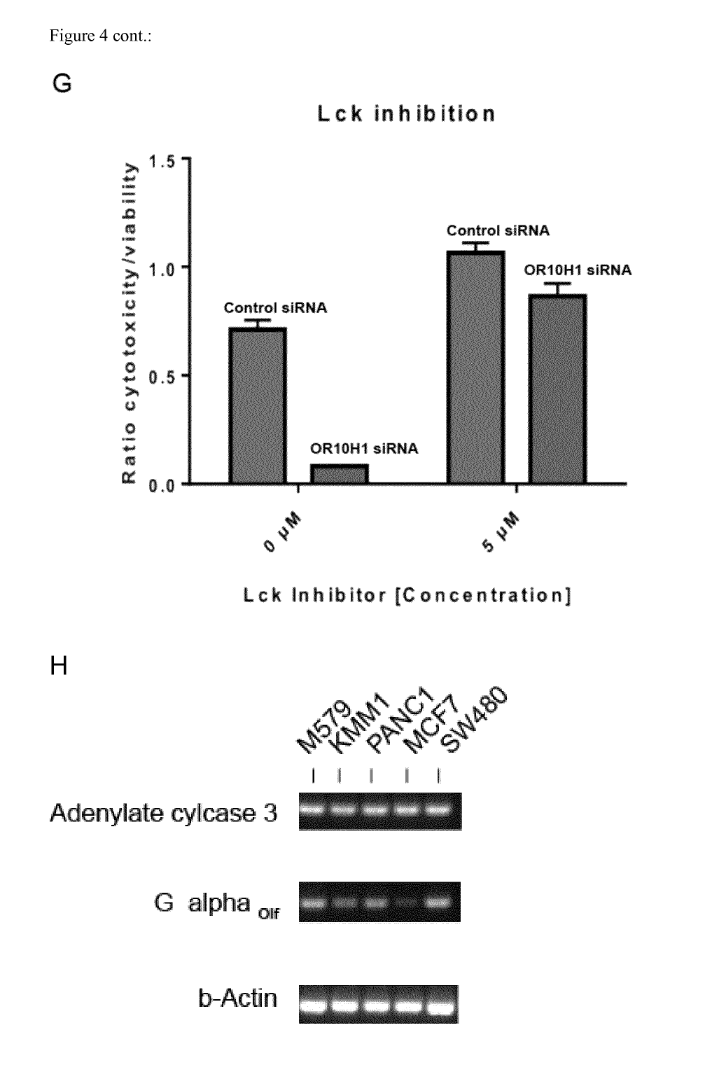

### OR10H1 Inhibits TIL Functionality Utilizing cAMP (FIG. 4)

TIL412 were co-cultured with (OR10H1 knock-down or negative siRNA control) M579-A2 for 10 h cells to activate signaling pathways in the TILs, and then the melanoma cells were then removed from the co-culture using magnetic melanoma beads specific for MCSP (Miltenyi Biotech). The purity of the remaining TILs was around 99.5% (not shown). Differential gene expression between the TILs isolated from the OR10H1 knock-down and the negative siRNA control was measured by RNA-Sequencing according to standard procedures. Several genes were significantly up or down regulated (fold change above 0.5 or below -0.5 respectively) in the setting with OR10H1 knock-down M579-A2 (siRNA 1) compared to the negative siRNA control (FIG. 4A). Down-regulated in the TILs were the following genes: Early growth response gene 3 (Egr3) is a key negative transcriptional regulator of T cell activation and induces anergy [1-3]. Interferon-gamma receptor chain 2 (IFNGR2) expressed in high levels inhibits T cell proliferation and may induce apoptosis [4, 5]. Nuclear Receptor Subfamily 4, Group A, Member 2 (NR4A2) is associated with T cell exhaustion in chronic viral infection [6] and might be involved in apoptosis [7]. Up-regulated in the TILs were the following genes: Chemokine (C-X-C motif) ligand 13 (CXCL13) promotes T cell recruitment [8] and facilitates the inflammatory response of antigen-experienced T helper cells [9]. Cytotoxic And Regulatory T Cell Molecule (CRTAM) is expressed (upon TCR activation) on activated T cells and its interaction with Nec1-2 (on tumors) promotes IFN-g secretion by CD8+ T cells [10]. Vav guanine nucleotide exchange factor 3 (VAV3) is involved in TCR signaling via NFAT and SRF activation [11]. FBJ Murine Osteosarcoma Viral Oncogene Homolog (FOS) together with c-Jun builds up activating protein 1 (AP-1), one of three major transcription factors downstream of TCR signaling [12]. V-Myc Avian Myelocytomatosis Viral Oncogene Homolog (c-Myc) is a transcription factor which is involved in proliferation and metabolic reprogramming upon T lymphocyte activation [13, 14]. It modulates the generation of CD8+ immunological memory in tumors and viral infection [15, 16]. Interferon regulatory factor 4 (IRF4) is a transcription factor necessary for the expansion and effector differentiation of CD8+ T cells and represses genes involved in cell cycle arrest and apoptosis. [17, 18]

Upon Ingenuity pathway analysis (Qiagen; FIG. 4B) to investigate functions in which the differential expressed genes are enriched (positive or negative activation) in TILs and which signaling networks play a role, the inventors surprisingly found that of the eight pathways analyzed, only CREB is differentially phosphorylated in the setting with OR10H1-negative⁻ melanoma. The log 2 ratio (compared to unstimulated TILs) for CREB is significantly reduced after the knock-down of OR10H1 (p=0.006; FIG. 4C).

Western blot analysis for phospho-CREB and ATFL (same phosphorylation site) in TIL412 confirmed the reduced activation depending on OR10H1 knock-down on melanoma cells (FIG. 4F). cAMP response element-binding protein (CREB) downstream of TCR activation is widely known to be important for proliferation and survival of T cells [19] but has been associated with T cell anergy as well [20]. Therefore, key factors of TCR signaling (Lck, LAT, CREB, ZAP-70, Syk, CD3e and ERK1/2) were analyzed.

Knock-down of OR10H1 on melanoma cells leads to decreased phosphorylation of CREB and Lck (inhibitory phospho-Tyrosin 505) in TILs (FIGS. 4D and 4E). Importantly however, OR10H1 does not play a role in modulating CD3e activation (phosphylation): CD3e does not show increased phosphorylation levels after co-culture with melanoma cells/TILs (regardless of OR10H1 knock-down) compared to unstimulated TILs, and the same is observed for and ERK1/2. Yet, over-stimulation with PMA and ionomycin does dramatically increased phosphorylation of ERK1/2 but does not affect CD3e.

Phosphorylation of Linker of activated T cells (LAT) slightly increases in TILs in the first 30 minutes of co-culture and stays stable up to two hours (for OR10H1-positive and OR10H1-negative melanoma cell/TIL co-cultures and over-activation PMA and ionomycin). Spleen tyrosine kinase (Syk) gets activated in 5 minutes and later on decreases below the level of unstimulated TILs in all three settings. Finally, phosphorylation of Zeta-chain-associated protein kinase 70 (ZAP70) increases in the first 5 minutes and stays stable for 2 h if co-cultured with melanoma cells/TILs (regardless of OR10H1 knock-down). Over-activation with PMA and ionomycin leads to an increased phosphorylation of ZAP70.

Co-culture of TIL412 with OR10H1-positive M579-A2 melanoma cells leads to a dramatically increased phosphorylation of CREB after 2 h in TILs (FIG. 4D). Interestingly, the first 30 min after activation the phosphorylation levels are similar to the OR10H1 knock-down setting. Over activation with PMA and ionomycin leads to a faster onset of CREB phosphorylation but the levels are similar to the control siRNA (OR10H1-positive) setup. In contrast, Lymphocyte-specific protein tyrosine kinase (Lck) becomes strongly dephosphorylated after 30 min in TILs if co-cultured with OR10H-negative M579-A2 melanoma cells or overactivated with PMA and ionomycin but not in the control siRNA (OR10H1-positive) setup (FIG. 4E).

As described above, TCRs of TILs which were co-cultured with melanoma cells were activated in the same way, but showed different signaling at two important hubs, namely CREB and Lck, depending on whether OR10H1 was present or absent on the target melanoma cells. Therefore, OR10H1-mediated inhibition of T cell activity might converge here. Lck has two phosphorylation sites but phosphoplex analysis of Lck measures total Lck phosphorylation. Phosphorylation of Lck-Tyr384 stabilizes the active conformation whereas phosphorylation of Lck-Tyr505 promotes the auto-inhibited conformation of Lck [21-23]. Western blots for phospho-Lck confirmed that phosphorylation of the inhibitory Tyr505 residue was strongly reduced after the knock-down of OR10H1 suggesting an increased activation of Lck in TILs. Indeed, inhibition of Lck activity by a small molecule inhibitor is shown to abrogate OR10H1 knock-down effect on T-cell mediated cytotoxicity (as reflected by the ratio of cytotoxicity/viability) of melanoma cells (FIG. 4G). Protein kinase A (PKA) is activated by cAMP and in turn activates c-Src Tyrosine Kinase (Csk). Csk abrogates Lck activity by phosphorylation of Lck-Tyr505 [24, 25]. Indeed, the inventors were able to confirm by western blot analysis, an enhancement of PKA phosphorylation in TILs after the OR10H1-positive (ie control siRNA) M579-A2 melanoma cell/TIL co-culture. (FIG. 4F).

Olfactory receptor signaling activates a unique G protein (G-alpha-Olf) and subsequently adenylate cyclase type III (FIG. 4H). RNA-sequencing expression data suggest that both genes are expressed in M579-A2. M579-A2 melanoma cells (OR10H1-positive and OR10H1-negative) were transiently transfected with a cAMP reporter luciferase construct and co-cultured with TIL412. The production of cAMP in melanoma is altered by OR10H1 knock-down only if there is an interaction with TILs, while co-culture of M579-A2 cells with TILs alone was not sufficient to trigger a cAMP response (not shown). Indeed, after co-culture of M579-A2 (OR10H1-positive or OR10H1-negative) with TILs for 2 h, 10 uM forskolin was added to raise cAMP levels: without a preceding co-culture with TILs there is no difference in cAMP response in M579-A2 cells, whereas in the presence of TILs the cAMP response in M579-A2 cells is reduced if OR10H1 is knocked-down on the melanoma cells (FIG. 41).

The inventors were surprised to observe data suggesting that that TIL-driven cytotoxicity of the melanoma cells may be inhibited by a mechanism involving connexin 32 (Cx32) and the transport of cAMP from the tumor cell to the T cell. We validated by RNA-sequencing that M579-A2 cells express connexin 32, a protein involved in the formation of gap-junctions. Such gap-junctions are selective for the transport of cAMP and cGMP [28], and hence the potential for cAMP transport from M579-A2 to the TIL412. Indeed, preliminary results suggest that blockade of Cx32 leads to an increased killing of melanoma cells by TILs only if OR10H1 is present (not shown). This is consistent with recent studies have shown that regulatory T cells (Tregs) and tumor cells can transport cAMP via gap-junctions into T cells and lead to inhibition through phosphorylation of Lck and CREB [26, 27].

### Example 5

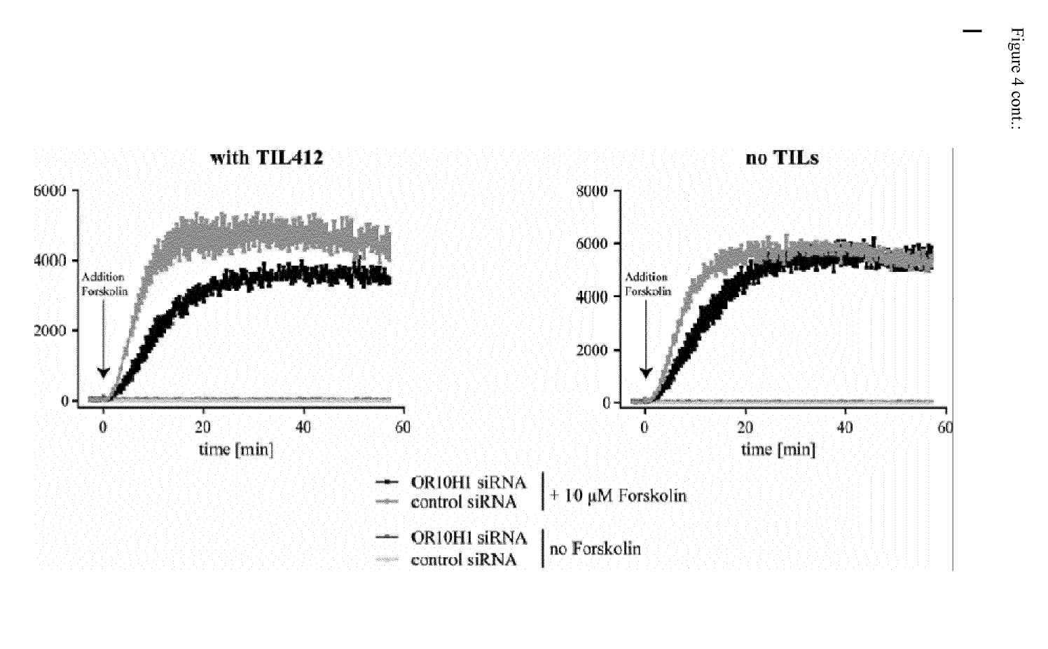

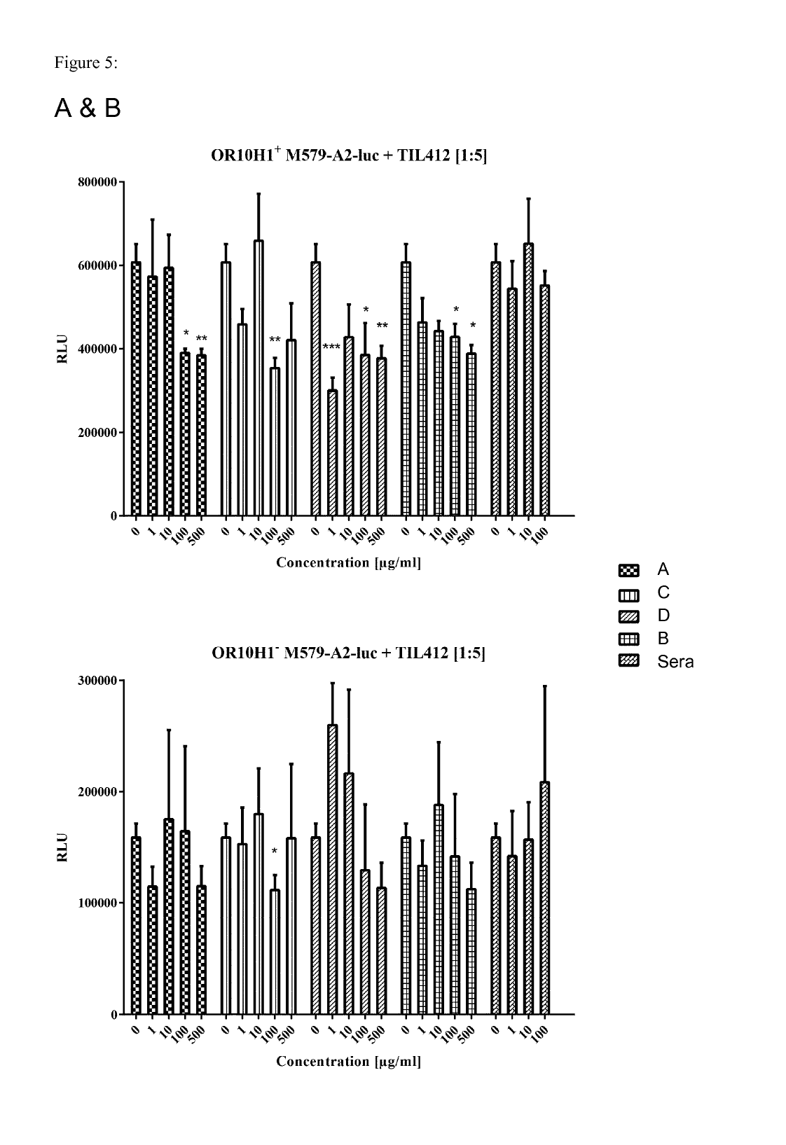

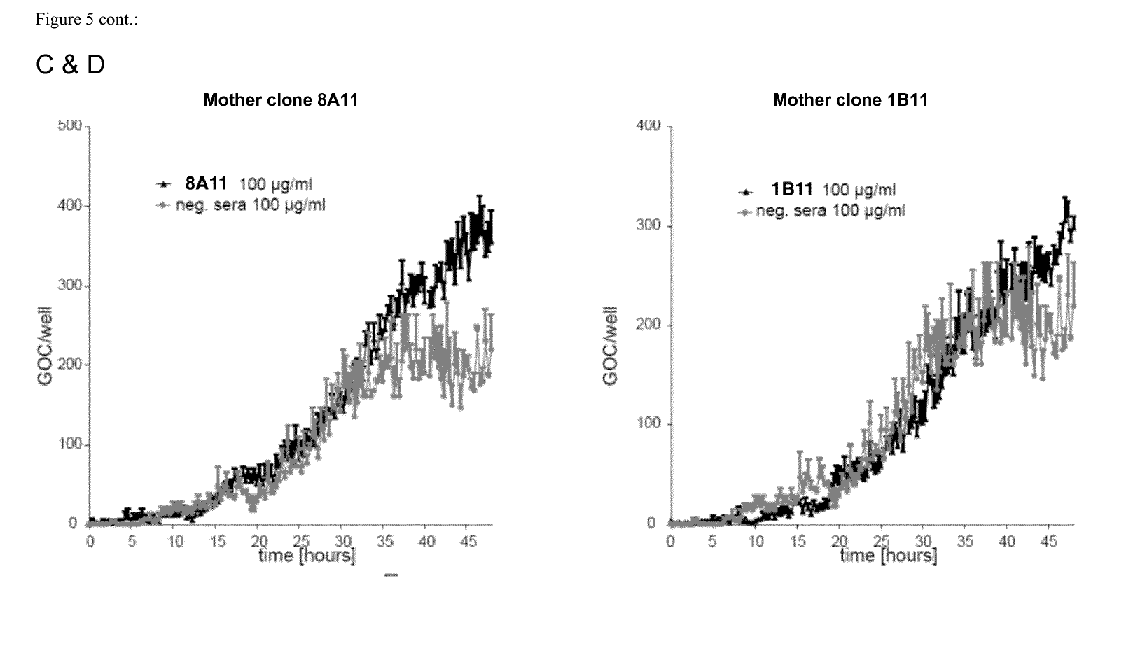

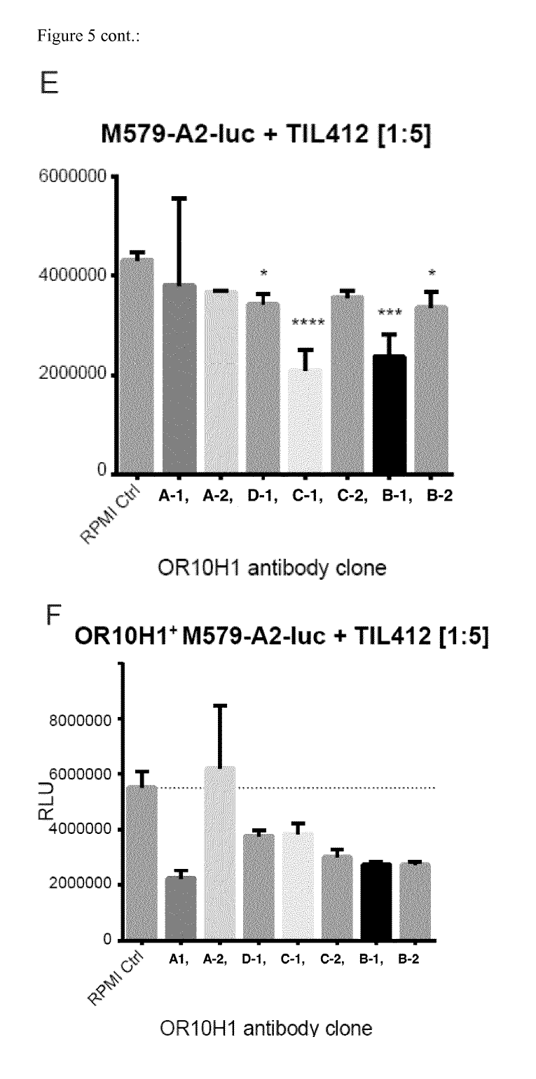

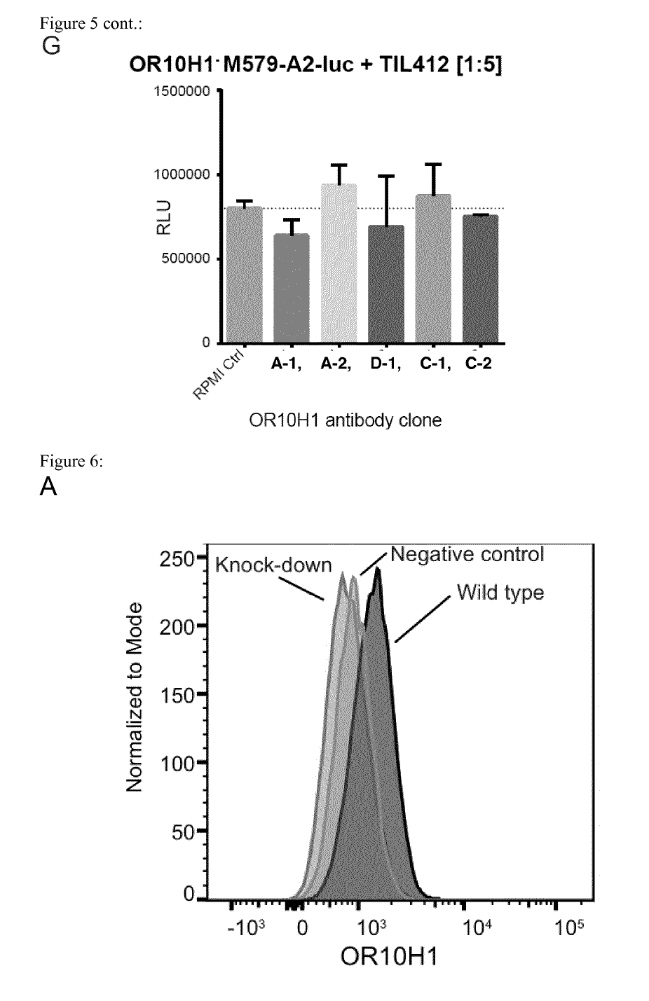

### Development of Antibodies Against OR10H1 and their Functional Activity (FIG. 5)

For generating antibodies against OR10H1, genomic immunization of rats was conducted at Aldevron (Freiburg), described briefly as follows.

Firstly, an OR10H1 construct was cloned into an Aldrevon immunization vector (pB8-OR10H1) and an Aldrevon screening vector (pB1-OR10H1). Transient transfection of these vectors into mammalian cells confirmed (weak) cell surface expression. Cell surface expression of such proteins—containing a vector-derived N-terminal tag-sequence—was analyzed by flow cytometry on non-fixed living cells using an anti-tag murine antibody and a goat anti-mouse IgG R-phycoerythrin conjugate (Southern Biotech) as a secondary antibody. Secondly, three rats were immunized with the immunization vector (pB8-OR10H1) for 46 days and 5 genetic applications (IS46d-5) followed by an additional 2 genetic immunizations over a subsequent 35 days; in total immunization over for 81 days with 7 genetic applications (IS81d-7)). Thirdly, sera of immunized rats (diluted in PBS 3% FBS) were then tested (by flow cytometry) for reactivity against mammalian cells transiently transfected with the screening vector (pB1-OR10H1) using a goat anti-rat IgG R-phycoerythrin conjugate (Southern Biotech) as a secondary antibody. Compared to pre-immunization sera, a significant (but weak reactivity) was observed (data not shown). Fourthly, splenocytes were isolated from the rats' spleen, the resulting B cells fused with immortalized myeloma cells by standard technologies, and the resulting hybridoma clones screened for the reactivity of their supernatant against the recombinant mammalian cells transiently transfected with the screening vector (as described above). Screening and enrichment of such hybridoma clones resulted in 4 mother clones being selected (1C3, 1B11, 8A11 and 4B4).

Each of these antibodies produced by such clones showed a significant impact on TIL-mediated melanoma killing at a concentration of 100 μg/ml in the OR10H1-positive M579-A1-Luc/TIL412 assay (FIGS. 5A and B) as well as for those tested (8A11 and 1B11) in the IncuCyte cytotoxicity assay based on caspase activation (FIGS. 5C and D), in each case as described above.

Next, the mother clones were single-cell sorted to generate individual hybridoma clones that produce monoclonal antibodies, which were tested for their functional activity in the OR10H1-positive M579-A1-Luc/TIL412 assay as described above (FIGS. 5E, F and G), and individual hybridoma clones were selected for sequencing of the antibody they produce.

Such results demonstrate the ability of antibodies of the present invention to have therapeutic and research utility as and/or in the development of compounds for the treatment of proliferative disease such as a cancer disease, in particular of OR10H1-positive cancers.

### Example 6

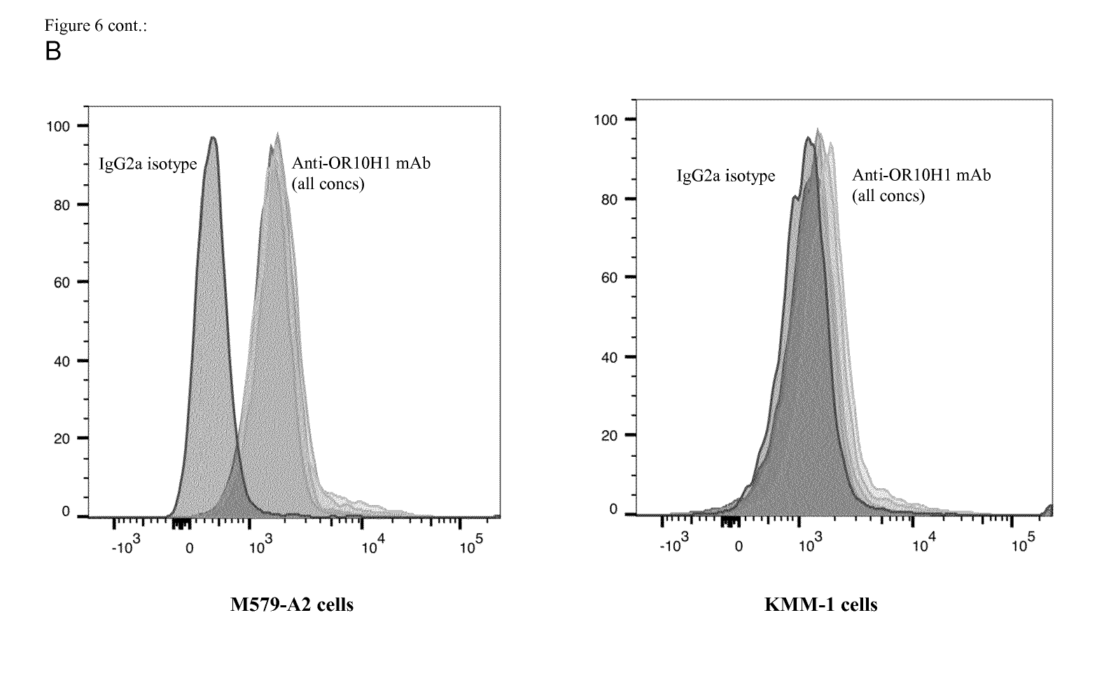

### Detection of OR10H1 using an Antibody of the Present Invention (FIG. 6)

The post-immunisation poly sera was used to demonstrate that it can specifically detect OR10H1 expressed on the surface of tumor cells. FACS detection of melanoma M579-A2 cells transiently transfected with negative control siRNA or OR10H1 knock-down siRNA1 was conducted (FIG. 6A), using a chicken anti-rat IgG-AlexaFluor 647 conjugate (ThermoFisher A-21472) as a secondary antibody. Cells knocked-down for OR10H1 show reduced fluorescence compared to wild-type (ie, OR10H1-positive) cells.

In particular, the antibody purified from clone Di-8A11-H12-E6 shows specificity (compared to control IgG2a isotype antibody) by binding to those melanoma cells (M579-A2) that express OR10H1 but not binding to myeloma cells (KMM-1) that do not express OR10H1 (FIG. 6B). OR10H1 expression is determined by RT-PCR.

Such results demonstrate the ability of antibodies of the present invention to detected the expression of OR10H1 protein and their utility to determine increased resistance of a cell against an immune response, such as of a cancer cell.

### Example 7

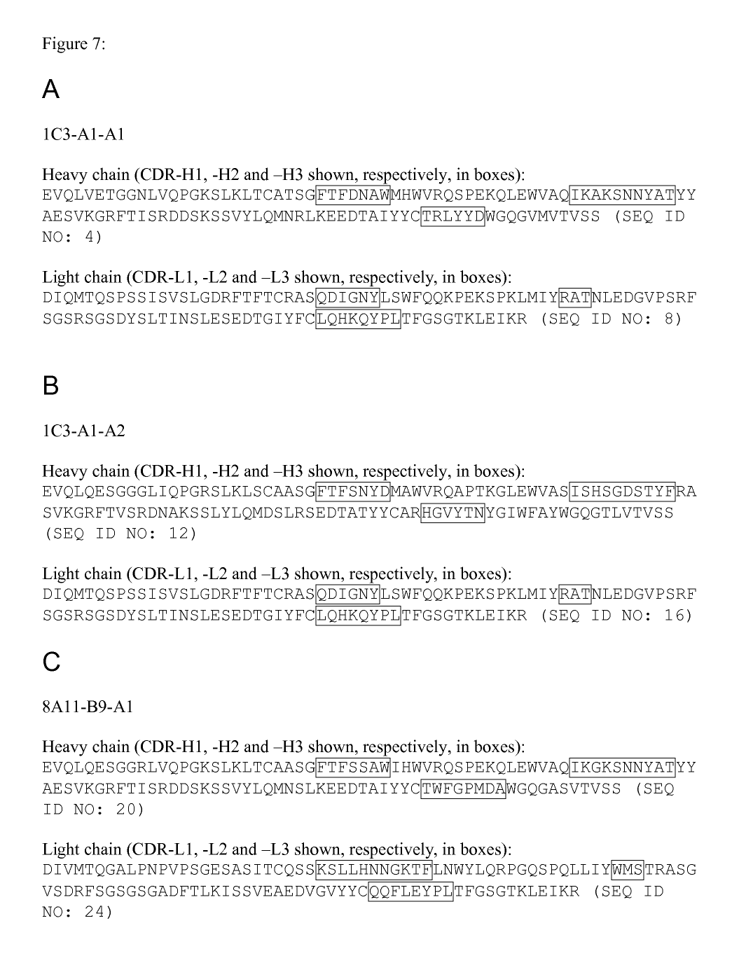

### Sequences of Antibodies of the Invention (FIG. 7)

The sequences of variable domains of the antibodies produced by hybridoma clones of Example 5 were determined by standard procedures at Antibody Designs Laboratories (San Diego). Briefly, the following general procedure was followed: (1) total RNA extraction and cDNA

Synthesis; (2) 5'RACE extension; (3) amplification of VH and VL domains including leader sequence and partial constant regions CH1 and CL; (4) cloning of PCR positive reactions; (5) colony PCR and sequencing of clones with proper insert size; and (6) sequencing analysis up to 5× coverage or 10 clones per chain. From the nucleic acid sequence the amino acid sequence of the variable domains of the chains of each antibody was determined. The amino acid sequence of the variable domains of heavy and light chain of exemplary antibodies of the invention is shown in FIGS. 7A, B and C. Hybridoma clone Di-8A11-H12-E6 is deposited at the DSMZ (Leibniz-Institut DSMZ—Deutsche Sammlung von Mikroorganismen and Zellkulturen GmbH, Inhoffenstr. 7B, 38124 Braunschweig, Germany) in accordance with the Budapest Treaty. (DSMZ Deposition Number: DSM ACC3310, deposited 26 Oct. 2016, Depositor: Deutsches Krebsforschungszentrum Stiftung des offentlichen Rechts, Im Neuenheimer Feld 280, 69120 Heidelberg, Germany). Hybridoma clone Di-8A11-H12-E6 is a hybridoma comprising Rat B lymphocytes fused with murine Ag8 myeloma cells, and can be cultured at 37° C. in a 5% CO₂ atmosphere in RPMI-1640 medium supplemented with 10% Fetal calf serum, 10 U/ml human recombinant IL-6 and 1% penicillin-streptomycin (10,000 U/mL). The optimal split ratio is 1:1. The viability of these hybridoma cells may be relatively low when first thawed, however this recovers over a few days in culture. Each antibody chain sequence comprises c-terminally a constant region (Table E3).

TABLE E3
Rat Constant Domain Regions:

| Rat IgG | Uniprot ID for constant-region | Constant region |
|---|---|---|
| subclass | (accessed 20 Oct. 2016) | length (aa) |
| IgG1 | P20759 (Sequence version 1 of 1 Feb. 1991; | 326 |
| | Entry version 113; 5 Oct. 2016) | |
| IgG2a | P20760 (Sequence version 1 of 1 Feb. 1991; | 322 |
| | Entry version 116; 5 Oct. 2016) | |
| IgG2b | P20761 (Sequence version 1 of 1 Feb. 1991; | 333 |
| | Entry version 105; 5 Oct. 2016) | |
| IgG2c | P20762 (Sequence version 1 of 1 Feb. 1991; | 329 |
| | Entry version 96; 5 Oct. 2016) | |
| IgG kappa | P01836 (Sequence version 1 of 21 Jul. 1986; | 106 |
| (A allele) | Entry version 80; 5 Oct. 2016) | |
| IgG kappa | P01835 (Sequence version 1 of 21 Jul. 1986; | 106 |
| (B allele) | Entry version 89; 5 Oct. 2016) | |

### Example 8

### Peptide Fingerprint Mapping of Antibodies of the Invention

Using standard procedures—briefly, trypsin digestion of antibody protein followed by peptide mass fingerprinting using MALDI-TOF mass spectrometry—(Toplab GmbH, Martinsried, Germany), the IgG subclass of the constant region of the antibody chain(s) is determined, by comparison of the peptide mass-peaks measured by MALDI-TOF mass spectrometry to the mass of peptide fragments predicted to be obtained from trypsin digestion of the respective rat IgG subclasses (eg Table E3).

### REFERENCES FOR EXAMPLES

1. Safford, M., et al., Egr-2 and Egr-3 are negative regulators of T cell activation. Nat Immunol, 2005. 6(5): p. 472-480.

2. Collins, S., et al., Opposing regulation of T cell function by Egr-1/NAB2 and Egr-2/Egr-3. European Journal of Immunology, 2008. 38(2): p. 528-536.

3. Zheng, Y., Y. Zha, and T. F. Gajewski, Molecular regulation of T-cell anergy. EMBO Rep., 2008. 9: p. 50-55.

4. Bernabei, P., et al., Interferon-γ receptor 2 expression as the deciding factor in human T, B, and myeloid cell proliferation or death. Journal of Leukocyte Biology, 2001. 70(6): p. 950-960.

5. Schroder, K., et al., Interferon-γ: an overview of signals, mechanisms and functions. Journal of Leukocyte Biology, 2004. 75(2): p. 163-189.

6. Wherry, E. J., et al., Molecular Signature of CD8+ T Cell Exhaustion during Chronic Viral Infection. Immunity, 2007. 27(4): p. 670-684.

7. Zhou, T., et al , Inhibition of Nur77/Nurr1 leads to inefficient clonal deletion of self-reactive T cells. J Exp Med, 1996. 183(4): p. 1879-92.

8. Hui, W., C. Zhao, and S. G. Bourgoin, LPA Promotes T Cell Recruitment through Synthesis of CXCL13. 2015. 2015: p. 1-10.

9. Manzo, A., et al., Mature antigen-experienced T helper cells synthesize and secrete the B cell chemoattractant CXCL13 in the inflammatory environment of the rheumatoid joint. Arthritis & Rheumatism, 2008. 58(11): p. 3377-3387.

10. Boles, K. S., et al., The tumor suppressor TSLC1/NECL-2 triggers NK-cell and CD8+ T-cell responses through the cell-surface receptor CRTAM. Blood, 2005. 106(3): p. 779-786.

11. Charvet, C., et al., Membrane localization and function of Vav3 in T cells depend on its association with the adapter SLP-76. Journal of Biological Chemistry, 2005. 280(15): p. 15289-15299.

12. Huang, Y. and R. L. Wange, T Cell Receptor Signaling: Beyond Complex Complexes. Journal of Biological Chemistry, 2004. 279(28): p. 28827-28830.

13. Dose, M., et al., c-Myc mediates pre-TCR-induced proliferation but not developmental progression. Blood, 2006. 108(8): p. 2669-2677.

14. Wang, R., et al., The Transcription Factor Myc Controls Metabolic Reprogramming upon T Lymphocyte Activation. Immunity, 2011. 35(6): p. 871-882.

15. Hague, M., et al., C-Myc regulation by costimulatory signals modulates the generation of CD8+ memory T cells during viral infection. Open Biology, 2016. 6(1).

16. Chandran, S. S., et al., Tumor-Specific Effector CD8+ T Cells That Can Establish Immunological Memory in Humans after Adoptive Transfer Are Marked by Expression of IL7 Receptor and c-myc. Cancer Research, 2015. 75(16): p. 3216-3226.

17. Yao, S., et al., Interferon Regulatory Factor 4 Sustains CD8+ T Cell Expansion and Effector Differentiation. Immunity, 2013. 39(5): p. 833-845.

18. Huber, M. and M. Lohoff, IRF4 at the crossroads of effector T-cell fate decision. European Journal of Immunology, 2014. 44(7): p. 1886-1895.

19. Wen, A. Y., K. M. Sakamoto, and L. S. Miller, The role of the transcription factor CREB in immune function. Journal of immunology (Baltimore, Md.: 1950), 2010. 185(11): p. 6413-6419.

20. Powell, J. D., et al., The-180 Site of the IL-2 Promoter Is the Target of CREB/CREM Binding in T Cell Anergy. The Journal of Immunology, 1999. 163(12): p. 6631-6639.

21. Abraham, N., et al., Enhancement of T-cell responsiveness by the lymphocyte-specific tyrosine protein kinase p561ck. Nature, 1991. 350(6313): p. 62-66.

22. Gervais, F. G., et al., The SH2 domain is required for stable phosphorylation of p561ck at tyrosine 505, the negative regulatory site. Molecular and Cellular Biology, 1993. 13(11): p. 7112-7121.

23. Yamaguchi, H. and W. A. Hendrickson, Structural basis for activation of human lymphocyte kinase Lck upon tyrosine phosphorylation. Nature, 1996. 384(6608): p. 484-489.

24. Vang, T., et al., Activation of the COOH-terminal Src kinase (Csk) by cAMP-dependent protein kinase inhibits signaling through the T cell receptor. The Journal of experimental medicine, 2001. 193(4): p. 497-507.

25. Taskén, K. and a. J. Stokka, The molecular machinery for cAMP-dependent immuno-modulation in T-cells. Biochemical Society transactions, 2006. 34(Pt 4): p. 476-479.

26. Bopp, T., et al., Cyclic adenosine monophosphate is a key component of regulatory T cell-mediated suppression. The Journal of Experimental Medicine, 2007. 204(6): p. 1303-1310.

27. Ye, J., et al., TLR8 signaling enhances tumor immunity by preventing tumor-induced T-cell senescence. EMBO Molecular Medicine, 2014. 6(10): p. 1294-1311.

28. Bevans, C. G., Isoform Composition of Connexin Channels Determines Selectivity among Second Messengers and Uncharged Molecules. Journal of Biological Chemistry, 1998. 273(5): p. 2808-2816.

1. A compound for use in the treatment of a disease of a subject, wherein the compound is a modulator of the expression, function and/or stability of Olfactory Receptor, Family 10, Subfamily H, Member 1 (OR10H1), or of a variant of OR10H1.

2. The compound for use according to claim 1, wherein the disease is characterized by a pathological immune response.

3. The compound for use according to claim 1 or 2, wherein the disease is characterized by expression of said OR10H1, or the variant of OR10H1.

4. The compound for use according to any one of claims 1 to 3, wherein the variant of OR10H1 is a protein comprising an amino acid sequence having at least 90% sequence identity to the sequence of SEQ ID NO: 25 (OR10H1 amino acid sequence).

5. The compound for use according to any one of claims 1 to 4, wherein the compound is an inhibitor or antagonist of expression, function and/or stability of OR10H1, or of the variant of OR10H1.

6. The compound for use according to any one of claims 1 to 5, wherein the compound is selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

7. The compound for use according to any one of claims 1 to 6, wherein the compound is an antigen binding construct (for example, an antibody, antibody-like molecule or other antigen binding derivative, or an antigen binding fragment thereof), that binds said OR10H1, or the variant of OR10H1.

8. The compound for use according to any one of claims 1 to 7, wherein the compound is an OR10H1 inhibitory antibody, or an inhibitory antigen binding fragment thereof, or an inhibitory antibody of a variant of OR10H1, or an inhibitory antigen binding fragment thereof.

9. The compound for use according to any one of claims 1 to 6, wherein the compound is a nucleic acid (for example an anti-sense nucleotide molecule such as a siRNA or shRNA molecule) that binds to a nucleic acid that encodes or regulates the expression of OR10H1, or of a variant of OR10H1.

10. The compound for use according to any one of claims 1 to 6, wherein the compound is a nucleic acid (for example an anti-sense nucleotide molecule such as a siRNA or shRNA molecule) that binds to a nucleic acid that encodes or regulates the expression of a gene that controls the expression, function and/or stability of OR10H1, or of a variant of OR10H1.

11. The compound for use according to any one of claim 1 to 6, 9 or 10, wherein the compound is a nucleic acid inhibitor of expression of OR10H1, or of a variant of OR10H1.

12. The compound for use according to any one of claims 1 to 11, wherein the disease is selected from a proliferative disease.

13. The compound for use according to any one of claims 1 to 12, wherein the disease is cancer, preferably a cancer disease selected from lung cancer, bladder cancer, ovarian cancer, uterine cancer, endometrial cancer, breast cancer, liver cancer, pancreatic cancer, stomach cancer, cervical cancer, lymphoma, leukemia, acute myeloid leukemia, acute lymphocytic leukemia, salivary gland cancer, bone cancer, brain cancer, colon cancer, rectal cancer, colorectal cancer, kidney cancer, skin cancer, melanoma, squamous cell carcinoma, pleomorphic adenoma, hepatocellular carcinoma, and/or adenocarcinoma.

14. The compound for use according to claim 13, wherein the cancer is an OR10H1 positive cancer, or a cancer positive for the variant of OR10H1.

15. The compound for use according to any one of claims 1 to 14, wherein the compound is for use in enhancing an immune response in the subject, preferably for use in aiding a cell-mediated immune response in the subject such as the subject's T-cell mediated immune response, for example for treating a proliferative disease such as a cancer disease.

16. The compound for use according to any one of claims 1 to 15, wherein the treatment comprises a transfer of cells to the subject, preferably a transfer of immune cells to the subject, more preferably an adoptive T-cell transfer.

17. The compound for use according to claim 16, wherein the cells are autologous cells of the subject, for example autologous immune cells, such as T-cells or Natural Killer (NK)-cells, of the subject.

18. The compound for use according to any one of claims 1 to 17, wherein the compound is an inhibitor or antagonist of expression, function and/or stability of said OR10H1, or the variant of OR10H1, and wherein the inhibition of the expression, function and/or stability of said OR10H1, or the variant of OR10H1, enhances an immune response, preferably enhances a cell-mediated immune response in the subject such as a T-cell mediated immune response in the subject, for example for treating a proliferative disease such as a cancer disease.

19. The compound for use according to claim 18, wherein the immune response is enhanced by an increase in T-cell activity and/or survival.

20. The compound for use according to claim 19, wherein the increase in T-cell activity and/or survival is associated with the inhibition of OR10H1-mediated or OR10H1-variant-mediated cAMP signaling.

21. The compound for use according to any one of claims 1 to 20, wherein the subject is a mouse, rat, guinea pig, rabbit, cat, dog, monkey, or preferably a human, for example a human patient.

22. The compound for use according to claim 21, wherein the subject is a human, for example a human patient.

23. The compound for use according to any one of claims 1 to 22, wherein the treatment comprises a step of administering a therapeutically effective amount of the compound to the subject.

24. An isolated antigen binding construct, capable of specifically binding to OR10H1, or of a variant of OR10H1, optionally wherein the antigen binding construct inhibits the expression, function and/or stability of OR10H1, or the variant of OR10H1.

25. The isolated antigen binding construct according to claim 24, which comprises a sequence of an antibody variable heavy and/or light chain of an antibody obtainable from hybridoma Di-8A11-H12-E6 (DSMZ Deposition Number: DSM ACC3310, deposited 26 Oct. 2016).

26. The isolated antigen binding construct according to claim 25, which is an antibody obtainable from hybridoma Di-8A11-H12-E6 (DSMZ Deposition Number: DSM ACC3310, deposited 26 Oct. 2016), or an antigen binding fragment obtainable from such antibody.

27. An isolated antigen binding construct comprising at least one Complementary Determining Region (CDR) 3 having an amino acid sequence with at least 80% sequence identity to an amino acid sequence selected from SEQ ID NOs. 3, 7, 11, 15, 19, and 23.

28. The isolated antigen binding construct according to claim 27, comprising an antibody variable chain sequence having at least 80% sequence identity to an amino acid sequence selected from SEQ ID Nos. 4, 8, 12, 16, 20, and 24.

29. The isolated antigen binding construct according to claim 27 or 28, comprising an antigen binding fragment of an antibody, wherein said antigen binding fragment comprises CDR1, CDR2 and CDR3, optionally selected from the CDR1, CDR2 and CDR3 sequences having the respective amino acid sequences of SEQ ID Nos. 1, 2, 3; or 5, 6, 7; or 9, 10, 11; or 13, 14, 15; or 17, 18, 19; or 21, 22, 23; in each case independently, optionally with not more than three or two, preferably one, amino acid substitution(s), insertion(s) or deletion(s) compared to these sequences.

30. The isolated antigen binding construct according to claim 29, wherein said CDR1 has an amino acid sequence of SEQ ID No 1, 5, 9, 13, 17 or 21, and CDR2 has an amino acid sequence of SEQ ID No 2, 6, 10, 14, 18, or 22, and CDR3 has an amino acid sequence of SEQ ID No 3, 7, 11, 15, 19, and 23; in each case independently, optionally with not more than three or two, preferably one, amino acid substitution(s), insertion(s) or deletion(s) compared to these sequences.

31. The isolated antigen binding construct according to any one of claims 27 to 30, wherein the antigen binding construct is an antibody, or an antigen binding fragment thereof, composed of at least one, preferably two, antibody heavy chain sequences, and at least one, preferably two, antibody light chain sequences, wherein at least one, preferably both, of said antibody heavy chain sequences comprise CDR1 to CDR3 sequences having the amino acid sequences of SEQ ID NO: 1 to 3, and at least one, preferably both, of said antibody light chain sequences comprise CDR1 to CDR3 sequences having the amino acid sequences of SEQ ID NO: 5 to 7; or wherein at least one, preferably both, of said antibody heavy chain sequences comprise CDR1 to CDR3 sequences having the amino acid sequences of SEQ ID NO: 9 to 11, and at least one, preferably both, of said antibody light chain sequences comprise CDR1 to CDR3 sequences having the amino acid sequences of SEQ ID NO: 13 to 15; or wherein at least one, preferably both, of said antibody heavy chain sequences comprise CDR1 to CDR3 sequences having the amino acid sequences of SEQ ID NO: 17 to 19, and at least one, preferably both, of said antibody light chain sequences comprises CDR1 to CDR3 sequences having the amino acid sequences of SEQ ID NO: 21 to 23; in each case of a CDR independently, optionally with not more than three or two, preferably one, amino acid substitution(s), insertion(s) or deletion(s) compared to these sequences.

32. The isolated antigen binding construct according to any one of claims 27 to 31, wherein the antigen binding construct is an antibody, or an antigen binding fragment thereof, composed of at least one, preferably two, antibody heavy chain sequence, and at least one, preferably two, antibody light chain sequence, wherein said antibody heavy chain sequence comprises a variable region having the amino acid sequence of SEQ ID NO: 4, and wherein said antibody light chain sequence comprises a variable region sequence having the amino acid sequence of SEQ ID NO: 8; or wherein said antibody heavy chain sequence comprises a variable region sequence having the amino acid sequence of SEQ ID NO: 12, and wherein said antibody light chain sequence comprises a variable region sequence having the amino acid sequence of SEQ ID NO: 16; or wherein said antibody heavy chain sequence comprises a variable region sequence having the amino acid sequence of SEQ ID NO: 20, and wherein said antibody light chain sequence comprises a variable region sequence having the amino acid sequence of SEQ ID NO: 24; in each case of a variable region sequence independently, optionally with not more than ten, nine, eight, seven, six, five, four, three, two or one, preferably not more than three, amino acid substitutions, insertions or deletions compared to these sequences.

33. An isolated antigen binding construct that competes for antigen binding to an isolated antigen binding construct according to any one of claims 24 to 32.

34. The compound for use according to any one of claims 1 to 23 or the isolated antigen binding construct according to any one of claims 24 to 33 which decreases resistance and/or enhances sensitivity of cells expressing OR10H1, or a variant of OR10H1, to an immune response.

35. The compound for use or the isolated antigen binding construct according to claim 34, wherein said an immune response is a cytotoxic immune response.

36. The compound for use or the isolated antigen binding construct according to claim 34 or 35, wherein the immune response is a cell-mediated immune response, in particular a T-cell-mediated immune response.

37. The compound for use or the isolated antigen binding construct according to any one of claims 34 to 36, wherein said immune response is lysis and/or killing of said cells mediated by cytotoxic T-cells.

38. The compound for use or the isolated antigen binding construct according to any one of claims 34 to 37, wherein said cells are cancer cells or have originated from a tumor cell.

39. The compound for use according to any one of claims 1 to 23 or the isolated antigen binding construct according to any one of claims 24 to 38 which increases T-cell activity and/or survival.

40. The compound for use according to any one of claims 1 to 23 or the isolated antigen binding construct according to any one of claims 24 to 39, wherein the compound enhances killing and/or lysis of cells expressing said OR10H1, or the variant of OR10H1.

41. The compound for use according to any one of claims 1 to 23, and 34 to 40, or the isolated antigen binding construct according to any one of claims 24 to 40, which is an anti-body or comprises an antibody variable heavy and/or light chain sequence, or is an antigen binding fragment thereof, wherein the construct is chimerized, optionally is humanized or murinized

42. The compound for use according to any one of claims 1 to 23 and 34 to 41, or the isolated antigen binding construct of any one of claims 24 to 41, which is a monoclonal antibody.

43. The compound for use according to any one of claims 1 to 23 and 34 to 42, or the isolated antigen binding construct of any one of claims 24 to 42, wherein said compound or antigen binding construct is an IgG antibody.

44. An isolated nucleic acid encoding for a compound according to any one of claims 41 to 43 or an antigen binding construct according to any one of claims 23 to 43, or encoding for a part or a monomer of an antigen binding construct.

45. A vector comprising a nucleic acid according to claim 44.

46. A recombinant host cell comprising a compound according to any one of claims 1 to 23 or 41 to 43, an antigen binding construct according to any one of claims 24 to 43, or a nucleic acid according to claim 44, or a vector according to claim 45.

47. The recombinant host cell according to claim 46, wherein the cell is a human cell, preferably an autologous human cell.

48. The recombinant host cell according to claim 46, wherein the cell is a Chinese hamster ovary (CHO) cell.

49. A pharmaceutical composition comprising the compound according to any one of claims 1 to 23 or 41 to 43, the isolated antigen binding construct according to any one of claims 24 to 43, or the isolated nucleic acid according to claim 44, or the vector according to claim 45, or the recombinant host cell according to claim 46 or 47; and a pharmaceutically acceptable carrier, stabilizer and/or excipient.

50. The compound according to any one of claims 1 to 23 or 41 to 43, or the isolated antigen binding construct according to any one of claims 24 to 43, or a isolated nucleic acid according to claim 44, or a vector according to claim 45, or a recombinant host cell according to claim 46 or 47, or the pharmaceutical composition according to claim 49, for use in medicine, preferably for use in the diagnosis, prevention, and/or treatment of a proliferative disease, for example a disease comprising a malignant or benign tumor disease, such as a cancer.

51. A method of producing a recombinant cell line capable of expressing an antigen binding construct specific for OR10H1, or for a OR10H1 variant, comprising
(a) providing a suitable host cell,
(b) providing at least one genetic construct comprising coding sequence(s) encoding the antigen binding construct according to any one of claims 24 to 43 or the compound according to any one of claims 41 to 43,
(c) introducing into said suitable host cell said genetic construct(s),
(d) optionally, expressing said genetic construct(s) by said suitable host cell under conditions that allow for the expression of the antigen binding construct.

52. A method of producing an antigen binding construct specific for OR10H1, or for a OR10H1 variant, comprising
(c) providing a hybridoma or host cell capable of expressing a compound of any one of claims 41 to 43 or an antigen binding construct according to any one of claims 24 to 43, for example a recombinant cell line comprising at least one genetic construct comprising coding sequence(s) encoding said compound or antigen binding construct,
(d) culturing said hybridoma or host cell under conditions that allow for the expression of the antigen binding construct.

53. A hybridoma or host cell capable of expressing a compound of any one of claims 41 to 43 or an antigen binding construct according to any one of claims claim 24 to 43.

54. A method for detecting an OR10H1 protein, or a variant of OR10H1, in a sample comprising contacting the sample with an antigen binding construct specifically binding to said OR10H1 protein, or to the variant of OR10H1, and detecting the binding between said antigen binding construct and said OR10H1 protein, or the variant of OR10H1.

55. The method according to claim 54, wherein the antigen binding construct is an antibody or an antigen binding fragment thereof.

56. The method according to claim 54 or 55, wherein the sample is a sample of a tumor or a cancer, preferably a tumor sample of a patient suffering from a refractory/recurrent tumor disease, a metastatic tumor disease, or a multidrug resistant tumor disease.

57. A method for diagnosing a resistance phenotype of a cancer disease against an immune response such as a cell-mediated immune response in a subject, the method comprising the steps of
(a) Providing a sample of tumor or cancer cells of the subject,
(b) Determining the presence or absence of protein or mRNA of OR10H1, or of a variant of OR10H1, in the tumor or cancer cells, and
(c) Diagnosing a resistance phenotype of the cancer disease against an immune response in the subject when protein or mRNA of said OR10H1, or of the variant of OR10H1, is present in the tumor or cancer cells.

58. A kit, comprising means for the detection of protein or mRNA of OR10H1, or of a variant of OR10H1, wherein said means is preferably selected from: (i) an antigen binding construct specifically binding said protein of OR10H1, or the variant of OR10H1, and/or (ii) a nucleic acid for detecting said mRNA of OR10H1, or of the variant of OR10H1; optionally together with instructions for use and/or with one or more other components useful for said detection.

59. The method according to claim 57 or the kit according to claim 58, wherein said means are coupled to a detectable label.

60. The kit according to claim 58 or 59, for use in the diagnosis of a resistance of a proliferative disease, for example a cancer disease, against an immune response such as a cell-mediated immune response; preferably the kit is for use in a method according to claim 57 or 59.

61. A method for identifying a compound suitable for the treatment of a disease characterized by expression of OR10H1, or a variant thereof, the method comprising the steps of
(a) Providing a first cell expressing a protein or mRNA of OR10H1, or of a variant of OR10H1, and
(b) Providing a candidate compound, and
(c) Optionally, providing a second cell which is a cytotoxic immune cell, for example a cytotoxic T-lymphocyte (CTL), capable of immunologically recognizing said first cell, and
(d) Bringing into contact the first cell and the candidate compound, and optionally the second cell, and
(e) Determining subsequent to step (d), either or both of:
(i) expression, function and/or stability of said protein or mRNA of OR10H1, or of a variant of OR10H1, in said first cell, wherein a reduced expression, function and/or stability of said protein or mRNA of OR10H1, or of a variant of OR10H1, in said first cell contacted with the candidate compound compared to said first cell not contacted with said candidate compound indicates that the candidate compound is a compound suitable for the treatment of a disease characterized by expression of said protein or mRNA of OR10H1, or of a variant of OR10H1; and/or
(ii) cytotoxicity of said second cell against said first cell, wherein an enhanced cytotoxicity of said second cell against said first cell contacted with the candidate compound compared to the cytotoxicity of said second cell against said first cell not contacted with the candidate compound indicates that the candidate compound is a compound suitable for the treatment of a disease characterized by expression of said OR10H1, or the variant of OR10H1.

62. The method according to claim 61, wherein said first cell is a tumor cell or a cell derived from a tumor cell.
